SEQUENCE LISTING (37 sequences)

SEQ ID NO: 1 - protein, 617 aa, synthetic construct
  REGION 1..617: note = Glucoamylase from Rasamsonia emersonii (Uniprot Q9C1V4) associated with the signal sequence of Saccharomyces cerevisiae alpha-mating factor 1
  SIGNAL 1..19
MRFPSIFTAVLFAASSALARAPVAARATGSLDSFLATETPIALQGVLNNIGPNGADVAGASAGIVVASPSRSDPNYFYSWTRDAALTAKYLVDAFIAGNKDLEQTIQQYISAQAKVQTISNPSGDLSTGGLGEPKFNVNETAFTGPWGRPQRDGPALRATALIAYANYLIDNGEASTADEIIWPIVQNDLSYITQYWNSSTFDLWEEVEGSSFFTTAVQHRALVEGNALATRLNHTCSNCVSQAPQVLCFLQSYWTGSYVLANFGGSGRSGKDVNSILGSIHTFDPAGGCDDSTFQPCSARALANHKVVTDSFRSIYAINSGIAEGSAVAVGRYPEDVYQGGNPWYLATAAAAEQLYDAIYQWKKIGSISITDVSLPFFQDIYPSAAVGTYNSGSTTFNDIISAVQTYGDGYLSIVEKYTPSDGSLTEQFSRTDGTPLSASALTWSYASLLTASARRQSVVPASWGESSASSVPAVCSATSATGPYSTATNTVWPSSGSGSSTTTSSAPCTTPTSVAVTFDEIVSTSYGETIYLAGSIPELGNWSTASAIPLRADAYTNSNPLWYVTVNLPPGTSFEYKFFKNQTDGTIVWEDDPNRSYTVPAYCGQTTAILDDSWQ

SEQ ID NO: 2 - DNA, 1854 bp, synthetic construct
  misc_feature 1..1854: note = Encoding SEQ ID NO: 1
  source 1..1854: mol_type = other DNA
atgaggttcccatctattttcaccgctgttttgtttgctgcttcttctgctttggctagagcaccagttgctgctagagctacaggttctttggattcttttttggctactgaaactccaatcgcattgcaaggtgttttgaacaatattggtccaaacggtgctgatgttgccggtgcttctgctggtatagttgttgcttctccatctagatctgatcccaattacttttactcttggactagagatgctgctttgactgctaaatacttggttgatgctttcattgccggtaacaaggatttggaacaaaccattcaacagtacatttccgctcaagctaaggttcaaactatctctaatccatccggtgatttgtctactggtggtttgggtgaacctaagttcaatgttaacgaaactgcttttactggtccatggggtagaccacaaagagatggtccagctttgagagctactgctttaattgcttacgccaactacttgatcgataacggtgaagcttctactgctgatgagattatttggccaatcgttcaaaacgacttgtcctacattactcagtactggaactcttctactttcgacttgtgggaagaagttgaaggttcttcttttttcactaccgccgttcaacatagagctttggttgaaggtaatgctttggctaccagattgaaccatacttgttctaactgtgtttcccaagctccacaagttttgtgtttcttgcaatcttattggaccggttcttacgttttggctaattttggtggttctggtagatctggtaaggatgttaattctatcttgggttccatccatacttttgatcctgctggtggttgtgatgattctacttttcaaccatgttctgcaagagcattggctaaccataaggttgttactgactctttcagatccatctacgctattaactctggtattgctgaaggttcagctgttgctgttggtagatatcctgaagatgtttaccaaggtggtaatccatggtatttggctacagctgctgctgcagaacaattatatgatgctatctaccagtggaagaagatcggttctatttccattaccgatgtgtctttgccattcttccaagacatatatccatctgctgcagttggtacttacaactctggttctactactttcaacgatatcatctctgctgttcaaacttacggtgatggttacttgtctatcgtcgaaaagtatactccatccgatggttctttgaccgaacaattttctagaactgatggtactccattgtctgcttcagctttgacttggtcttatgcttctttgttgacagcttctgctagaaggcaatctgttgttccagcttcttggggtgaatcttctgcttcttcagttccagctgtttgttctgctacatctgctactggtccttattctacagctactaatactgtttggccatcttctggttcaggttcttcaactactacttcttctgctccttgtactactccaacttcagttgctgttactttcgatgagatcgtttctacctcttatggtgaaactatctacttggctggttccattccagaattaggtaattggtctactgcttccgctattccattgagagctgatgcttacactaattctaacccattgtggtacgttaccgttaatttgccaccaggtacatcttttgagtacaagtttttcaagaaccagaccgatggtactatcgtttgggaagatgatccaaacagatcttatactgttccagcatactgtggtcaaactaccgctattttggatgattcttggcagtga

SEQ ID NO: 3 - protein, 598 aa, Rasamsonia emersonii
RAPVAARATGSLDSFLATETPIALQGVLNNIGPNGADVAGASAGIVVASPSRSDPNYFYSWTRDAALTAKYLVDAFIAGNKDLEQTIQQYISAQAKVQTISNPSGDLSTGGLGEPKFNVNETAFTGPWGRPQRDGPALRATALIAYANYLIDNGEASTADEIIWPIVQNDLSYITQYWNSSTFDLWEEVEGSSFFTTAVQHRALVEGNALATRLNHTCSNCVSQAPQVLCFLQSYWTGSYVLANFGGSGRSGKDVNSILGSIHTFDPAGGCDDSTFQPCSARALANHKVVTDSFRSIYAINSGIAEGSAVAVGRYPEDVYQGGNPWYLATAAAAEQLYDAIYQWKKIGSISITDVSLPFFQDIYPSAAVGTYNSGSTTFNDIISAVQTYGDGYLSIVEKYTPSDGSLTEQFSRTDGTPLSASALTWSYASLLTASARRQSVVPASWGESSASSVPAVCSATSATGPYSTATNTVWPSSGSGSSTTTSSAPCTTPTSVAVTFDEIVSTSYGETIYLAGSIPELGNWSTASAIPLRADAYTNSNPLWYVTVNLPPGTSFEYKFFKNQTDGTIVWEDDPNRSYTVPAYCGQTTAILDDSWQ

SEQ ID NO: 4 - DNA, 1797 bp, synthetic construct
  misc_feature 1..1797: note = Encoding SEQ ID NO: 3
  source 1..1797: mol_type = other DNA
agagcaccagttgctgctagagctacaggttctttggattcttttttggctactgaaactccaatcgcattgcaaggtgttttgaacaatattggtccaaacggtgctgatgttgccggtgcttctgctggtatagttgttgcttctccatctagatctgatcccaattacttttactcttggactagagatgctgctttgactgctaaatacttggttgatgctttcattgccggtaacaaggatttggaacaaaccattcaacagtacatttccgctcaagctaaggttcaaactatctctaatccatccggtgatttgtctactggtggtttgggtgaacctaagttcaatgttaacgaaactgcttttactggtccatggggtagaccacaaagagatggtccagctttgagagctactgctttaattgcttacgccaactacttgatcgataacggtgaagcttctactgctgatgagattatttggccaatcgttcaaaacgacttgtcctacattactcagtactggaactcttctactttcgacttgtgggaagaagttgaaggttcttcttttttcactaccgccgttcaacatagagctttggttgaaggtaatgctttggctaccagattgaaccatacttgttctaactgtgtttcccaagctccacaagttttgtgtttcttgcaatcttattggaccggttcttacgttttggctaattttggtggttctggtagatctggtaaggatgttaattctatcttgggttccatccatacttttgatcctgctggtggttgtgatgattctacttttcaaccatgttctgcaagagcattggctaaccataaggttgttactgactctttcagatccatctacgctattaactctggtattgctgaaggttcagctgttgctgttggtagatatcctgaagatgtttaccaaggtggtaatccatggtatttggctacagctgctgctgcagaacaattatatgatgctatctaccagtggaagaagatcggttctatttccattaccgatgtgtctttgccattcttccaagacatatatccatctgctgcagttggtacttacaactctggttctactactttcaacgatatcatctctgctgttcaaacttacggtgatggttacttgtctatcgtcgaaaagtatactccatccgatggttctttgaccgaacaattttctagaactgatggtactccattgtctgcttcagctttgacttggtcttatgcttctttgttgacagcttctgctagaaggcaatctgttgttccagcttcttggggtgaatcttctgcttcttcagttccagctgtttgttctgctacatctgctactggtccttattctacagctactaatactgtttggccatcttctggttcaggttcttcaactactacttcttctgctccttgtactactccaacttcagttgctgttactttcgatgagatcgtttctacctcttatggtgaaactatctacttggctggttccattccagaattaggtaattggtctactgcttccgctattccattgagagctgatgcttacactaattctaacccattgtggtacgttaccgttaatttgccaccaggtacatcttttgagtacaagtttttcaagaaccagaccgatggtactatcgtttgggaagatgatccaaacagatcttatactgttccagcatactgtggtcaaactaccgctattttggatgattcttggcagtga

SEQ ID NO: 5 - protein, 19 aa, synthetic construct
  REGION 1..19: note = Signal sequence of Saccharomyces cerevisiae alpha-mating factor 1
MRFPSIFTAVLFAASSALA

SEQ ID NO: 6 - protein, 618 aa, Rasamsonia emersonii
  SIGNAL 1..20
MASLVAGALCILGLTPAAFARAPVAARATGSLDSFLATETPIALQGVLNNIGPNGADVAGASAGIVVASPSRSDPNYFYSWTRDAALTAKYLVDAFIAGNKDLEQTIQQYISAQAKVQTISNPSGDLSTGGLGEPKFNVNETAFTGPWGRPQRDGPALRATALIAYANYLIDNGEASTADEIIWPIVQNDLSYITQYWNSSTFDLWEEVEGSSFFTTAVQHRALVEGNALATRLNHTCSNCVSQAPQVLCFLQSYWTGSYVLANFGGSGRSGKDVNSILGSIHTFDPAGGCDDSTFQPCSARALANHKVVTDSFRSIYAINSGIAEGSAVAVGRYPEDVYQGGNPWYLATAAAAEQLYDAIYQWKKIGSISITDVSLPFFQDIYPSAAVGTYNSGSTTFNDIISAVQTYGDGYLSIVEKYTPSDGSLTEQFSRTDGTPLSASALTWSYASLLTASARRQSVVPASWGESSASSVPAVCSATSATGPYSTATNTVWPSSGSGSSTTTSSAPCTTPTSVAVTFDEIVSTSYGETIYLAGSIPELGNWSTASAIPLRADAYTNSNPLWYVTVNLPPGTSFEYKFFKNQTDGTIVWEDDPNRSYTVPAYCGQTTAILDDSWQ

SEQ ID NO: 7 - DNA, 1854 bp, synthetic construct; identical to SEQ ID NO: 2
  misc_feature 1..1854: note = Encoding SEQ ID NO: 6
  source 1..1854: mol_type = other DNA

SEQ ID NO: 8 - protein, 20 aa, synthetic construct
  REGION 1..20: note = Signal sequence associated with Rasamsonia emersonii glucoamylase from(Uniprot Q9C1V4)
MASLVAGALCILGLTPAAFA

SEQ ID NO: 9 - protein, 508 aa, synthetic construct
  REGION 1..508: note = Glucoamylase from Saccharomycopsis fibuligera associated with the signal sequence of Saccharomyces cerevisiae alpha-mating factor 1
  SIGNAL 1..19
MRFPSIFTAVLFAASSALANTGHFQAYSGYTVNRANFTQWIHEQPAVSWYYLLQNIDYPEGQFKAAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

SEQ ID NO: 10 - DNA, 1527 bp, synthetic construct
  misc_feature 1..1527: note = Encoding SEQ ID NO: 9
  source 1..1527: mol_type = other DNA
atgaggttcccatctattttcaccgctgttttgtttgctgcttcttctgctttggctaacaccggtcatttccaagcttattctggttataccgttaacagagctaacttcacccaatggattcatgaacaaccagctgtttcttggtactacttgttgcaaaacatcgattacccagaaggtcaattcaaagctgctaaaccaggtgttgttgttgcttctccatctacatctgaaccagattacttctaccaatggactagagataccgctattaccttcttgtccttgattgctgaagttgaagatcattctttctccaacactaccttggctaaggttgtcgaatattacatttccaacacctacaccttgcaaagagtttctaatccatccggtaacttcgattctccaaatcatgatggtttgggtgaacctaagttcaacgttgatgatactgcttatacagcttcttggggtagaccacaaaatgatggtccagctttgagagcttacgctatttctagatacttgaacgctgttgctaagcacaacaacggtaaattattattggccggtcaaaacggtattccttattcttctgcttccgatatctactggaagattattaagccagacttgcaacatgtttctactcattggtctacctctggttttgatttgtgggaagaaaatcaaggtactcatttcttcaccgctttggttcaattgaaggctttgtcttacggtattccattgtctaagacctacaatgatccaggtttcacttcttggttggaaaaacaaaaggatgccttgaactcctacattaactcttccggtttcgttaactctggtaaaaagcacatcgttgaatctccacaattgtcatctagaggtggtttggattctgctacttatattgctgccttgatcacccatgatatcggtgatgatgatacttacaccccattcaatgttgataactcctacgttttgaactccttgtattacctattggtcgacaacaagaacagatacaagatcaacggtaactacaaagctggtgctgctgttggtagatatcctgaagatgtttacaacggtgttggtacttctgaaggtaatccatggcaattggctactgcttatgctggtcaaactttttacaccttggcctacaattccttgaagaacaagaagaacttggtcatcgaaaagttgaactacgacttgtacaactccttcattgctgatttgtccaagattgattcttcctacgcttctaaggattctttgactttgacctacggttccgataactacaagaacgttatcaagtccttgttgcaattcggtgactcattcttgaaggttttgttggatcacatcgatgacaacggtcaattgactgaagaaatcaacagatacaccggttttcaagctggtgcagtttctttgacttggtcatctggttctttgttgtctgctaatagagccagaaacaagttgatcgaattattgtga

SEQ ID NO: 11 - protein, 616 aa, synthetic construct
  REGION 1..616: note = Glucoamylase from Rasamsonia emersonii (Uniprot A0A0F4YWQ6) associated with the signal sequence of Saccharomyces cerevisiae alpha-mating factor 1
  SIGNAL 1..19
MRFPSIFTAVLFAASSALARAPVAARASGSLDSFLATETPIALQGVLNNIGPNGADVAGASAGIVVASPSRSDPDYFYSWTRDAALTAKYLVDAFIAGNKDLEQTIQEYISAQAQVQTISNPSGDLSTGGLGEPKFNVNETAFTGPWGRPQRDGPALRATALIAYANYLIDNGQASTADEIIWPIVQNDLSYVTQYWNSSTFDLWEEVEGSSFFTTAVQHRALVEGNALATRLNHTCPNCVSQAPQVLCFLQSYWTGSYVLANFGGSGRSGKDVNSILGSIHTFDPAGGCDDSTFQPCSARALANHKVVTDSFRSVYAVNSGIAEGSAVAVGRYPEDVYQGGNPWYLATAAAAEQLYDAIYQWNKIGSISITDVSLAFFQDIYPSAAVGTYNSGSSTFNDIISAVQTYADGYLSIIEKYTPSDGSLTEQFSRSDGTPLSASGLTWSYASLLTAAARRQSIVPASWGESSASSVPAVCSATSATGPYSTATNTAWPSSGSGPSTTTSVPCTTPTSVAVTFDEIVSTTYGETIYLAGSIPELGNWSPSSAIPLRADAYTSSNPLWYVTLNLPAGTSFEYKFFKKETDGTIVWEDDPNRSYTVPAYCGQTTAILDDSWQ

SEQ ID NO: 12 - DNA, 1848 bp, synthetic construct
  misc_feature 1..1848: note = Encoding SEQ ID NO: 11
  source 1..1848: mol_type = other DNA
atgaggttcccatctattttcaccgctgttttgtttgctgcttcttctgctttggctagagcgcccgttgccgctagagcttcaggtagtttagatagttttctggctactgagacacctattgcattgcaaggggttctaaacaacattggaccgaacggggcagacgtggctggtgcttccgcagggatcgtggttgcgtctccatcaagaagcgaccctgactacttttatagttggacgagagatgcggcattaacagcgaagtatttagttgatgcattcatcgccggaaataaggatttagagcagactatacaagaatatatctcagcgcaagcacaagtccaaacgatatccaacccatcaggggacttgtcaacaggaggtctgggtgagccgaagtttaatgtgaacgagactgcgttcactgggccttggggtcgtccccagagggatgggccagctttgcgtgccacggctctgattgcttacgcaaattaccttatcgacaatggtcaggcaagtaccgctgatgaaataatttggccgattgtccagaacgacctttcatacgttacccagtattggaactcttccacattcgatttgtgggaggaagtcgaaggatccagcttctttacaactgccgttcagcacagggctttggttgaaggtaatgccttggcaacgcgtttgaaccatacgtgccctaattgcgtgtcccaagctccacaggttttgtgtttcctacagagctattggactggctcctacgtcctagcgaattttggcggcagtgggagatccggaaaagacgtgaacagcatcttggggagcattcacactttcgacccagctgggggctgcgatgatagtacgtttcagccatgttccgcccgtgcattggcgaatcataaggtggtaactgacagttttaggtcagtatacgcggttaattccgggattgctgaagggagtgctgtagcggtgggaaggtatccagaggacgtttatcagggtggtaatccctggtacctggctaccgccgctgctgctgaacaactgtatgacgccatttatcagtggaacaaaattggttctatatccatcaccgacgtctcactagcctttttccaggatatatatccaagcgccgctgtcggaacatacaactccggttcatcaacgttcaatgatataatctccgctgtgcaaacctacgcggacggttacttatctattatcgaaaaatacacgccaagtgacggtagcctgactgagcaatttagtcgttctgacggtactccattatctgcaagcggactaacgtggagctacgcaagcctacttacagcggccgctcgtagacaatcaattgtcccagccagttggggtgagagttctgcgagttctgttcctgcggtgtgtagtgcgacgtcagcgaccggcccttatagcaccgcaacaaatacggcttggccgagttccgggtctggtccaagcacaacgaccagtgtcccctgcacaacccctacatcagtggccgtcacttttgacgaaatcgtcagtacgacgtacggtgaaactatttatcttgcggggtcaataccggagctagggaattggtcaccgagttccgcgattcctttaagggcagacgcgtataccagtagcaacccgttgtggtatgttacacttaatctacccgcgggcactagcttcgagtacaaattctttaaaaaagaaacggacggcacgattgtttgggaggacgacccaaaccgttcttacactgtaccggcttattgcggccagacaacagcgatcttagacgacagttggcag

SEQ ID NO: 13 - protein, 597 aa, Rasamsonia emersonii
RAPVAARASGSLDSFLATETPIALQGVLNNIGPNGADVAGASAGIVVASPSRSDPDYFYSWTRDAALTAKYLVDAFIAGNKDLEQTIQEYISAQAQVQTISNPSGDLSTGGLGEPKFNVNETAFTGPWGRPQRDGPALRATALIAYANYLIDNGQASTADEIIWPIVQNDLSYVTQYWNSSTFDLWEEVEGSSFFTTAVQHRALVEGNALATRLNHTCPNCVSQAPQVLCFLQSYWTGSYVLANFGGSGRSGKDVNSILGSIHTFDPAGGCDDSTFQPCSARALANHKVVTDSFRSVYAVNSGIAEGSAVAVGRYPEDVYQGGNPWYLATAAAAEQLYDAIYQWNKIGSISITDVSLAFFQDIYPSAAVGTYNSGSSTFNDIISAVQTYADGYLSIIEKYTPSDGSLTEQFSRSDGTPLSASGLTWSYASLLTAAARRQSIVPASWGESSASSVPAVCSATSATGPYSTATNTAWPSSGSGPSTTTSVPCTTPTSVAVTFDEIVSTTYGETIYLAGSIPELGNWSPSSAIPLRADAYTSSNPLWYVTLNLPAGTSFEYKFFKKETDGTIVWEDDPNRSYTVPAYCGQTTAILDDSWQ

SEQ ID NO: 14 - DNA, 1791 bp, synthetic construct
  misc_feature 1..1791: note = Encoding SEQ ID NO: 13
  source 1..1791: mol_type = other DNA
agagcgcccgttgccgctagagcttcaggtagtttagatagttttctggctactgagacacctattgcattgcaaggggttctaaacaacattggaccgaacggggcagacgtggctggtgcttccgcagggatcgtggttgcgtctccatcaagaagcgaccctgactacttttatagttggacgagagatgcggcattaacagcgaagtatttagttgatgcattcatcgccggaaataaggatttagagcagactatacaagaatatatctcagcgcaagcacaagtccaaacgatatccaacccatcaggggacttgtcaacaggaggtctgggtgagccgaagtttaatgtgaacgagactgcgttcactgggccttggggtcgtccccagagggatgggccagctttgcgtgccacggctctgattgcttacgcaaattaccttatcgacaatggtcaggcaagtaccgctgatgaaataatttggccgattgtccagaacgacctttcatacgttacccagtattggaactcttccacattcgatttgtgggaggaagtcgaaggatccagcttctttacaactgccgttcagcacagggctttggttgaaggtaatgccttggcaacgcgtttgaaccatacgtgccctaattgcgtgtcccaagctccacaggttttgtgtttcctacagagctattggactggctcctacgtcctagcgaattttggcggcagtgggagatccggaaaagacgtgaacagcatcttggggagcattcacactttcgacccagctgggggctgcgatgatagtacgtttcagccatgttccgcccgtgcattggcgaatcataaggtggtaactgacagttttaggtcagtatacgcggttaattccgggattgctgaagggagtgctgtagcggtgggaaggtatccagaggacgtttatcagggtggtaatccctggtacctggctaccgccgctgctgctgaacaactgtatgacgccatttatcagtggaacaaaattggttctatatccatcaccgacgtctcactagcctttttccaggatatatatccaagcgccgctgtcggaacatacaactccggttcatcaacgttcaatgatataatctccgctgtgcaaacctacgcggacggttacttatctattatcgaaaaatacacgccaagtgacggtagcctgactgagcaatttagtcgttctgacggtactccattatctgcaagcggactaacgtggagctacgcaagcctacttacagcggccgctcgtagacaatcaattgtcccagccagttggggtgagagttctgcgagttctgttcctgcggtgtgtagtgcgacgtcagcgaccggcccttatagcaccgcaacaaatacggcttggccgagttccgggtctggtccaagcacaacgaccagtgtcccctgcacaacccctacatcagtggccgtcacttttgacgaaatcgtcagtacgacgtacggtgaaactatttatcttgcggggtcaataccggagctagggaattggtcaccgagttccgcgattcctttaagggcagacgcgtataccagtagcaacccgttgtggtatgttacacttaatctacccgcgggcactagcttcgagtacaaattctttaaaaaagaaacggacggcacgattgtttgggaggacgacccaaaccgttcttacactgtaccggcttattgcggccagacaacagcgatcttagacgacagttggcag

SEQ ID NO: 15 - protein, 617 aa, Rasamsonia emersonii
  SIGNAL 1..20
MASLVAGALCVLGLTPAAFARAPVAARASGSLDSFLATETPIALQGVLNNIGPNGADVAGASAGIVVASPSRSDPDYFYSWTRDAALTAKYLVDAFIAGNKDLEQTIQEYISAQAQVQTISNPSGDLSTGGLGEPKFNVNETAFTGPWGRPQRDGPALRATALIAYANYLIDNGQASTADEIIWPIVQNDLSYVTQYWNSSTFDLWEEVEGSSFFTTAVQHRALVEGNALATRLNHTCPNCVSQAPQVLCFLQSYWTGSYVLANFGGSGRSGKDVNSILGSIHTFDPAGGCDDSTFQPCSARALANHKVVTDSFRSVYAVNSGIAEGSAVAVGRYPEDVYQGGNPWYLATAAAAEQLYDAIYQWNKIGSISITDVSLAFFQDIYPSAAVGTYNSGSSTFNDIISAVQTYADGYLSIIEKYTPSDGSLTEQFSRSDGTPLSASGLTWSYASLLTAAARRQSIVPASWGESSASSVPAVCSATSATGPYSTATNTAWPSSGSGPSTTTSVPCTTPTSVAVTFDEIVSTTYGETIYLAGSIPELGNWSPSSAIPLRADAYTSSNPLWYVTLNLPAGTSFEYKFFKKETDGTIVWEDDPNRSYTVPAYCGQTTAILDDSWQ

SEQ ID NO: 16 - protein, 20 aa, synthetic construct
  REGION 1..20: note = Signal sequence associated with Rasamsonia emersonii glucoamylase (Uniprot A0A0F4YWQ6)
MASLVAGALCVLGLTPAAFA

SEQ ID NO: 17 - protein, 471 aa, Rhizomucor pusillus
  SIGNAL 1..21
MKFSISLSAAIVLFAAATSLASPLPQQQRYAKRATSDDWKSKAIYQLLTDRFGRADDSTSNCSNLSNYCGGTYEGITKHLDYISGMGFDAIWISPIPKNSDGGYHGYWATDFYQLNSNFGDESQLKTLIQAAHERGMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPPLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTANYDTSSDLYQFIKTVNSVRMKSNKTVYMDIYVGDNAYAFKHGDALVVLNNYGSGSTNQVSFSVSGKFDSGASLMDIVSNITTTVSSDGTVTFNLKDGLPAIFTSA

SEQ ID NO: 18 - protein, 615 aa, Aspergillus luchuensis
  SIGNAL 1..21
MDGWWIISLLVVTLGFSTVNAASRDQWIGRSIYQIVTDRFARSDNSTTAACDAAQGNYCGGSFQGIINKLDYIHDLGFDAVGLALQSSVPHTFCNSSHIQVSRYGSLQRKPRFPPEQQISQFIAYHGYWPNDLYSINSHFGTPKELQALSSALHNRGMYLMLDIVVGDMAWAGNSSTVDYSTFNPFDDEKYFHDFKLLSSDPTNETCVLDCWMGDTVVSLPDLRNEDDQVQNILGSWISGLVSNYSIDGLRIDSVLNIAPDFFSNFTKSSGVFTIGEGATADAADVCPLQPSLNGLLNYPFLSRVDLLRYYILTNAFNTTNGNLSTITESISYTKGQCEDVLALGTFTANQDVPRFGSYTSDISQYAGGRHPHPYVPIPTPHRRQDFSDTTDDQNTVYYGEEQHLTGSYNPVNREALWLTNYSMHSTSLPALVKSLNRLRSYASGDGEQYTQNSQSGSDYLSYLSAPIYNSTHILATRKGFAGNQVVSVVSNLGAKPASKAATKITLGSDETGFQSKQNVTEILSCKTYVTDSSGNLAVDLSSDGGPRVYYPTDSLKDSTDICDDQTKSATPSSSAASSVSPNQSKGSETCLFGVPLGISTLVVTVAMATSYAFI

SEQ ID NO: 19 - protein, 549 aa, Aspergillus oryzae
  SIGNAL 1..23
MVSSSSLGRFAVLVTSLVGSAVAATTAEWKSRSVYQTMTDRFARTDGSTTAPCNTTQGLYCGGTWRGTIDKLDYIQGMGFDAVMISPIVENIEGRVSYGEAYHGYWPLDLYSLNSHFGTHQDLLDLSEALHSRGMYLMMDTVINNMAYMTNGKDPAKNIDYSVFTPFNDSSYFHPYCKITDWNNYTNAQLCQTGDDKVALPDLFTEHEDVQQILEKWAKEIISTYSIDGLRIDAAKHVNPGFLKNFGDAIGAFMTGEVLQQEVDTICKYQNNYIGSVPNYPIYYSVLKAFTLGNTTDLANQVEIMKNSCDDVTALASFSENHDVARFASMTDDMALAKNVLTFTILYDGVPMIYQGQEQHLDGPGTPDNREAIWLTKYNTDAELYKLIAKLNTIRKHAYKLDPNYVSLQTYPIFRGGSELGFRKGVEGRQVVMLLSTQGSNSSAYNLTLPVSFNGGVQVMDVLNCVNYTVNPQSELIVPMDKGEPRVFFPTSLMPGSGLCGYTTANVSFVELKTKGAAAAMSLGAKTTSSAAHGVLLSVLLSSLVAVLL

SEQ ID NO: 20 - protein, 515 aa, Homo sapiens
  SIGNAL 1..19
MLLQAFLFLLAGFAAKISAQYSPNTQQGRTSIVHLFEWRWVDIALECERYLAPKGFGGVQVSPPNENVAIYNPFRPWWERYQPVSYKLCTRSGNEDEFRNMVTRCNNVGVRIYVDAVINHMCGNAVSAGTSSTCGSYFNPGSRDFPAVPYSGWDFNDGKCKTGSGDIENYNDATQVRDCRLTGLLDLALEKDYVRSKIAEYMNHLIDIGVAGFRLDASKHMWPGDIKAILDKLHNLNSNWFPAGSKPFIYQEVIDLGGEPIKSSDYFGNGRVTEFKYGAKLGTVIRKWNGEKMSYLKNWGEGWGFVPSDRALVFVDNHDNQRGHGAGGASILTFWDARLYKMAVGFMLAHPYGFTRVMSSYRWPRQFQNGNDVNDWVGPPNNNGVIKEVTINPDTTCGNDWVCEHRWRQIRNMVIFRNVVDGQPFTNWYDNGSNQVAFGRGNRGFIVFNNDDWSFSLTLQTGLPAGTYCDVISGDKINGNCTGIKIYVSDDGKAHFSISNSAEDPFIAIHAESKL

SEQ ID NO: 21 - protein, 555 aa, Aspergillus niger
  SIGNAL 1..25
MVSMSALRHGLGVLYLASWLGSSLAASTEQWKSRSIYQTMTDRFARTDGSTTSPCNTTEGLYCGGTWRGMINHLDYIQGMGFDAVMISPIIENVEGRVEYGEAYHGYWPVDLYSLNSHFGTHQDLLDLSDALHARDMYLMMDTVINNMAYITNGSDPATHIDYSTLTPFNSSSYYHPYCKITDWNNFTNAQLCQTGDNIVALPDLYTEHAEVQETLSNWAKEVISTYSIDGLRIDAAKHVNPGFLKNFGDALDIFMTGEVLQQEVSTICDYQNNYIGSLPNYPVYYAMLKAFTLGNTSALATQVQSMKNSCNDVTALSSFSENHDVARFASMTHDMALAKNILTFTLLFDGVPMIYQGQEQHLDGPGSPENREAIWLSEYNTDAELYKLIGKLNAIRKHAYRLDNHYPDVETYPIFEGGSELGFRKGIEGRQVVMLLSTQGTNSSAYNLSMPVSFTGGTVVTEILNCVNYTVNTQSELVVPMDKGEPRVFFPADLMPGSGLCGLPVANVTYAALRTQGAAAAEAALSLGIKTDAASSALLSLGLSVVAGLIVGMW

SEQ ID NO: 22 - protein, 567 aa, Aspergillus niger
  SIGNAL 1..22
MDDGWWIISLLVVTLGIPTVNAASRDQWIGRSIYQIVTDRFARSDNSTTAACDAALGNYCGGSFQGIINKLDYIQELGFDAIWISPAQSQISARTADLSAYHGYWPNDLYSINSHFGTPKELEALSSALHDRGMYLMLDIVVGDMAWAGNHSTVDYSNFNPFNDQKFFHDFKLLSSDPTNETCVLDCWMGDTVVSLPDLRNEDQQVQNILGTWISGLVSNYSIDGLRIDSVLNIAPDFFSNFTKSSGVFTVGEGATADAADVCPLQPSLNGLLNYPLYYILTDAFNTTNGNLSTITESISYTKGQCEDVLALGTFTANQDVPRFGSYTSDISLARNILTSSMLTDGIPILYYGEEQHLTGSYNPVNREALWLTNYSMRSTSLPTLVQSLNRLRSYASGDGEQYTQKSQSGSDYLSYLSAPIYNSTHILATRKGFAGNQIVSVVSNLGAKPASKATTKITLGSDETGFQSKQNVTEILSCKTYVTDSSGNLAVDLSSDGGPRVYYPTDSLKDSTDICGDQTKSATPSSSAASSASLTQSKGSETCLFGVPLGISTLVVTVAMATSYVF

SEQ ID NO: 23 - protein, 550 aa, Aspergillus niger
  SIGNAL 1..29
MMFRKSASLLGQRLMAVCLLCWCVSLATAASTEEWKTRSIYQTMTDRFALTNGSTTAPCNTTVANYCGGSWQGTIDKLDYIQGMGFDAIMISPVIKNIAGRSKDGEAYHGYWPLDLYEINSHFGTREELLKLSEEIHARGMYLLLDVVINNMAYMTDGEDPATTIDYNVFPQFNGSSYFHPYCLITNWNNYTDAQWCQTGDNYTALPDLYTEHTAVQNILMDWSKSVISNYSVDGLRIDAAKSLTPSFLPTYASTVGGFMTGEVMDSNATNVCKYQTDYLPSLPNYPLYYSMITAFLNGEPATLLEEIATINDLCPDTFAMVNFIEDQDVDRWAYMNDDIMLAKTALTFMMLYDGIPLVYQGLEQAIAYSNRAALWLTDFDTNATLYKHIKKLNAIRKHAINLDSSYISSKTYPIYQGGSELAFWKGNNGRQVIMVLSTAGSNGSAYTLTLPVSYGASEVVTEVLNCVNYTVNTYSQLVVDMDKGEPRVFFPASMMPGSGLCGYNTSNVTYSELRLAAVGSSSSAGSHSVIPSAFASLFMAIVAFLAFRA

SEQ ID NO: 24 - protein, 524 aa, Aspergillus niger
  SIGNAL 1..16
MTIFLFLAIFVATALAATPAEWRSQSIYFLLTDRFARTDNSTTASCDLSARQYCGGSWQGIINQLDYIQGMGFTAIWITPVTAQIPQDTGYGQAYHGYWQQDAYALNSHYGTADDLKALASALHSRGMYLMVDVVANHMGHNGTGSSVDYSVYRPFNSQKYFHNLCWISDYNNQTNVEDCWLGDNTVALPDLDTTSTEVKNMWYDWVESLVSNYSVDGLRVDTVKNVQKNFWPGYNNASGVYCIGEVFDGDASYTCPYQEDLDGVLNYPMYYPLLRAFESTNGSISDLYNMINTVKSTCRDSTLLGTFVENHDNPRFANYTSDMSLAKNAATFTILADGIPIIYAGQEQHYSGGNDPYNREATWLSGYKTTSELYTHIAASNKIRTHAIKQDTGYLTYKNYPIYQDTSTLAMRKGYNGTQTITVLSNLGASGSSYTLSLPGTGYTAGQKITEIYTCTNLTVNSNGSVPVPMKSGLPRILYPADKLVNGSSFCSSAITVFKDAGGGVLFFSYTVIFAQVLIAIMT

SEQ ID NO: 25 - protein, 632 aa, Aspergillus fischeri
  SIGNAL 1..23
MKWISPLLPLSLSLCLLGQAAHALTPAEWRSQSIYFLLTDRFGREDNSTTAACDVTQRLYCGGSWQGIINHLDYIQGMGFTAIWITPVTQQFYENTGDGTSYHGYWQQNIYEVNSNYGTAQDLRKLADALHARGMYLMVDVVANHMGYDGAGNSVDYSVFTPFDSSTYFHTYCLISDYNNQNNVEDCWLGDTTVSLPDLDTTNTAVRTIWYDWVKGLVANYSIDGLRIDTVKHVEKDFWPDYNDAAGVYCVGEVFSGDPSYTCPYQNYMDGVLNYPIYYQLLYAFQSTSGSISNLYNMISSVDSDCADPTLLGNFIENHDNPRFASYTSDYSQAKNVISFMFFSDGIPIVYAGQEQHYSGGADPANREAVWLSGYSTSATLYSWIASTNKIRKLAISKDSAYITSKNNPFYYDSNTLAMRKGSVAGSQVITVLSNKGSSGSSYTLSLSGTGYSAGATLVEMYTCTTLTVDSSGNLAVPMASGLPRVLVPSSWVSGSGLCGDSISTIATTTTSTTKTTTVATTTACASATALPILFEELVTTTYGETIYLTGSISQLGNWDTSSAIALSASKYTSSNPEWYATVTLPVGTSFQYKFFKKESDGSIVWESDPNRSYTVPAGCAGTTVTVSDTWR

SEQ ID NO: 26 - protein, 640 aa, Aspergillus luchuensis
  SIGNAL 1..21
MRVSTSSIALAVSLFGKLALGLSAAEWRTQSIYFLLTDRFGRTDNSTTATCNTGDQIYCGGSWQGIINHLDYIQGMGFTAIWISPITEQLPQDTSDGEAYHGYWQQKIYYVNSNFGTADDLKSLSDALHARGMYLMVDVVPNHMGYAGNGNDVDYSVFDPFDSSSYFHPYCLITDWDNLTMVQDCWEGDTIVSLPDLNTTETAVRTIWYDWVADLVSNYSVDGLRIDSVEEVEPDFFPGYQEAAGVYCVGEVDNGNPALDCPYQKYLDGVLNYPIYWQLLYAFESSSGSISNLYNMIKSVASDCSDPTLLGNFIENHDNPRFASYTSDYSQAKNVLSYIFLSDGIPIVYAGEEQHYSGGDVPYNREATWLSGYDTSAELYTWIATTNAIRKLAISADSDYITYKNDPIYTDSNTIAMRKGTSGSQIITVLSNKGSSGSSYTLTLSGSGYTSGTKLIEAYTCTSVTVDSNGDIPVPMASGLPRVLLPASVVDSSSLCGGSGNTTTTTTAATSTSKATTSSSSSSAAATTSSSCTATSTTLPITFEELVTTTYGEEVYLSGSISQLGEWHTSDAVKLSADDYTSSNPEWSVTVSLPVGTTFEYKFIKVDEGGSVTWESDPNREYTVPECGSGSGETVVDTWR

SEQ ID NO: 27 - protein, 576 aa, Gloeophyllum trabeum
  SIGNAL 1..17
MYRFLVCALGLLGTVLAQSVDSYVGSEGPIAKAGVLANIGPNGSKASGAAAGVVVASPSKSDPDYWYTWTRDSSLVFKSLIDQYTTGIDSTSSLRSLIDSFVIAEANIQQVSNPSGTLTTGGLGEPKFNVDETAFTGAWGRPQRDGPALRATALITYGNWLLSNGNTTWVTSTLWPIIQNDLNYVVQYWNQTTFDLWEEVNSSSFFTTAVQHRALREGAAFATKIGQTSSVSSYTTQAANLLCFLQSYWNPTSGYITANTGGGRSGKDANTLLASIHTYDPSAGCDATTFQPCSDKALSNLKVYVDSFRSVYSINSGIASNAAVATGRYPEDSYQGGNPWYLTTFAVAEQLYDALNVWAAQGSLNVTSISLPFFQQFSSSVTAGTYASSSTTYTTLTSAIKSFADGFVAINAQYTPSNGGLAEQFSRSNGSPVSAVDLTWSYASALTAFEARNNTQFAGWGAVGLTVPTSCSSNSGGGGGSTVAVTFNVNAQTVWGENIYITGSVDALSNWSPDNALLLSSANYPTWSITVNLPASTAIQYKYIRKNNGAVTWESDPNNSITTPASGSVTENDTWR

SEQ ID NO: 28 - protein, 632 aa, Trichoderma reesii
  SIGNAL 1..20
MHVLSTAVLLGSVAVQKVLGRPGSSGLSDVTKRSVDDFISTETPIALNNLLCNVGPDGCRAFGTSAGAVIASPSTIDPDYYYMWTRDSALVFKNLIDRFTETYDAGLQRRIEQYITAQVTLQGLSNPSGSLADGSGLGEPKFELTLKPFTGNWGRPQRDGPALRAIALIGYSKWLINNNYQSTVSNVIWPIVRNDLNYVAQYWNQTGFDLWEEVNGSSFFTVANQHRALVEGATLAATLGQSGSAYSSVAPQVLCFLQRFWVSSGGYVDSNINTNEGRTGKDVNSVLTSIHTFDPNLGCDAGTFQPCSDKALSNLKVVVDSFRSIYGVNKGIPAGAAVAIGRYAEDVYYNGNPWYLATFAAAEQLYDAIYVWKKTGSITVTATSLAFFQELVPGVTAGTYSSSSSTFTNIINAVSTYADGFLSEAAKYVPADGSLAEQFDRNSGTPLSALHLTWSYASFLTATARRAGIVPPSWANSSASTIPSTCSGASVVGSYSRPTATSFPPSQTPKPGVPSGTPYTPLPCATPTSVAVTFHELVSTQFGQTVKVAGNAAALGNWSTSAAVALDAVNYADNHPLWIGTVNLEAGDVVEYKYINVGQDGSVTWESDPNHTYTVPAVACVTQVVKEDTWQS

SEQ ID NO: 29 - protein, 574 aa, Trametes cingulata
  SIGNAL 1..22
MRFTLLTSLLGLALGAFAQSSAADAYVASESPIAKAGVLANIGPSGSKSNGAKAGIVIASPSTSNPNYLYTWTRDSSLVFKALIDQFTTGEDTSLRTLIDEFTSAEAILQQVPNPSGTVSTGGLGEPKFNIDETAFTDAWGRPQRDGPALRATAIITYANWLLDNKNTTYVTNTLWPIIKLDLDYVASNWNQSTFDLWEEINSSSFFTTAVQHRALREGATFANRIGQTSVVSGYTTQANNLLCFLQSYWNPTGGYITANTGGGRSGKDANTVLTSIHTFDPAAGCDAVTFQPCSDKALSNLKVYVDAFRSIYSINSGIASNAAVATGRYPEDSYMGGNPWYLTTSAVAEQLYDALIVWNKLGALNVTSTSLPFFQQFSSGVTVGTYASSSSTFKTLTSAIKTFADGFLAVNAKYTPSNGGLAEQYSRSNGSPVSAVDLTWSYAAALTSFAARSGKTYASWGAAGLTVPTTCSGSGGAGTVAVTFNVQATTVFGENIYITGSVPALQNWSPDNALILSAANYPTWSSTVNLPASTTIEYKYIRKFNGAVTWESDPNNSITTPASGTFTQNDTWR

SEQ ID NO: 30 - protein, 579 aa, Athelia rolfsil
  SIGNAL 1..18
MFRSLLALAACAVASVSAQSASATAYLTKESAVAKNGVLCNIGSQGCMSEGAYSGIVIASPSKTSPDYLYTWTRDSSLVFKMLIDQYTNGLDTTLRTLIDEFVSAEATIQQTSNSSGTVSTGGLGEPKFNIDETAFTGAWGRPQRDGPALRATAIMTYATYLYNNGNTSYVTNTLWPIIKLDLDYVNSDWNQTTFDLWEEVDSSSFFTTAVQHRALVQGAAFATLIGQTSSASTYSATAPSILCFLQSYWNTNGYWTANTGGGRSGKDANTILASIHTFDASAGCSAATSQPCSDVALANLKVYVDSFRSIYTINSGISSTSGVATGRYPEDSYYNGNPWYLCTLAVAEQLYDALIVWKAAGELNVTSVSLAFFQQFDSSITAGTYASSSSVYTSLISDIQAFADEFVDIVAKYTPSSGFLSEQYDKSTGAQDSAANLTWSYAAAITAYQARNGFTGASWGAKGVSTSCSTGATSPGGSSGSVEVTFDVYATTVYGQNIYITGDVSELGNWTPANGVALSSANYPTWSATIALPADTTIQYKYVNIDGSTVIWEDAISNREITTPASGTYTEKDTWDES

SEQ ID NO: 31 - protein, 604 aa, Rhizopus oryzae
  SIGNAL 1..25
MQLFNLPLKVSFFLVLSYFSLLVSAASIPSSASVQLDSYNYDGSTFSGKIYVKNIAYSKKVTVIYADGSDNWNNNGNTIAASYSAPISGSNYEYWTFSASINGIKEFYIKYEVSGKTYYDNNNSANYQVSTSKPTTTTATATTTTAPSTSTTTPPSRSEPATFPTGNSTISSWIKKQEGISRFAMLRNINPPGSATGFIAASLSTAGPDYYYAWTRDAALTSNVIVYEYNTTLSGNKTILNVLKDYVTFSVKTQSTSTVCNCLGEPKFNPDASGYTGAWGRPQNDGPAERATTFILFADSYLTQTKDASYVTGTLKPAIFKDLDYVVNVWSNGCFDLWEEVNGVHFYTLMVMRKGLLLGADFAKRNGDSTRASTYSSTASTIANKISSFWVSSNNWIQVSQSVTGGVSKKGLDVSTLLAANLGSVDDGFFTPGSEKILATAVAVEDSFASLYPINKNLPSYLGNSIGRYPEDTYNGNGNSQGNSWFLAVTGYAELYYRAIKEWIGNGGVTVSSISLPFFKKFDSSATSGKKYTVGTSDFNNLAQNIALAADRFLSTVQLHAHNNGSLAEEFDRTTGLSTGARDLTWSHASLITASYAKAGAPAA

SEQ ID NO: 32 - protein, 612 aa, Aspergillus oryzae
  SIGNAL 1..19
MVSFSSCLRALALGSSVLAVQPVLRQATGLDTWLSTEANFSRQAILNNIGADGQSAQGASPGVVIASPSKSDPDYFYTWTRDSGLVMKTLVDLFRGGDADLLPIIEEFISSQARIQGISNPSGALSSGGLGEPKFNVDETAFTGAWGRPQRDGPALRATAMISFGEWLVENSHTSIATDLVWPVVRNDLSYVAQYWSQSGFDLWEEVQGTSFFTVAVSHRALVEGSSFAKTVGSSCPYCDSQAPQVRCYLQSFWTGSYIQANFGGGRSGKDINTVLGSIHTFDPQATCDDATFQPCSARALANHKVVTDSFRSIYAINSGRAENQAVAVGRYPEDSYYNGNPWFLTTLAAAEQLYDALYQWDKIGSLAITDVSLPFFKALYSSAATGTYASSTTVYKDIVSAVKAYADGYVQIVQTYAASTGSMAEQYTKTDGSQTSARDLTWSYAALLTANNRRNAVVPAPWGETAATSIPSACSTTSASGTYSSVVITSWPTISGYPGAPDSPCQVPTTVSVTFAVKATTVYGESIKIVGSISQLGSWNPSSATALNADSYTTDNPLWTGTINLPAGQSFEYKFIRVQNGAVTWESDPNRKYTVPSTCGVKSAVQSDVWR

SEQ ID NO: 33 - protein, 630 aa, Ophiostoma floccosum
  SIGNAL 1..18
MKLSSLLPLAFLGQAVNALSPAEWRKQSIYFLLTDRFGRTDNSTSATCNTGDRAYCGGSWQGVINHLDYIQGMGFTAIWITPVTGQFYESTGDGTSYHGYWQQDIYSLNSHLGDQNDLKALSAALHARGMYLMVDVVANHMGYDGAGSNVDYSVFDAFPSSSYFHSYCEISNYDDQSNVEDCWLGDTTVSLPDLNTELTSVRSIWNSWVAGLVANYSIDGLRIDTVKHVETSFWPGYNDAAGVYCVGEVFDGDPAYTCAYQNYMDGVLNYPIYYQLLSAFESTSGSISNLYNMIKSVASDCADPTLLGNFIENHDNPRFASYTSDYSLAQNAISFLFFSDGIPIVYSGQEQHYSGGADPANREATWLSGYSTTATLYKHIKTTNQIRSLIIGKDSSWATSANSPFYQDSNTIAMLKGSASGSKVLTVLSNKGASGSSYTLSLGSTGYSSGASLVELYSCTTVTVDSSGNVPVPMASGLPRVLVPSSWVSGSGLCGTAVTTGTATATGTSTKATTATATTATSCTAATAVSVVFNELATTTYGENVYIIGSTSQLGSWSTANAIALSSSDYTSSNPLWHVTVSLPAGSSFTYKFIKKESDGTFVWESDPNRSYTVPTGCSGLSATVSATWR

SEQ ID NO: 34 - protein, 620 aa, Trichocladium griseum
  SIGNAL 1..19
MHTFSKLLVLGSAVQSALGRPHGSSRLQERAAVDTFINTEKPIAWNKLLANIGPNGKAAPGAAAGVVIASPSRTDPPYFFTWTPDAALVLTGIIESLGHNYNTTLQQVSNPSGTFADGSGLGEAKFNVDLTAFTGEWGRPQRDGPPLRAIALIQYAKWLIANGYKSTAKSVVWPVVKNDLAYTAQYWNETGFDLWEEVPGSSFFTIASSHRALTEGAYLAAQLDTECPPCTTVAPQVLCFQQAFWNSKGNYVVSTSTAGEYRSGKDANSILASIHNFDPEAGCDNLTFQPCSERALANHKAYVDSFRNLYAINKGIAQGKAVAVGRYSEDVYYNGNPWYLANFAAAEQLYDAIYVWNKQGSITVTSVSLPFFRDLVSSVSTGTYSKSSSTFTNIVNAVKAYADGFIEVAAKYTPSNGALAEQYDRNTGKPDSAADLTWSYSAFLSAIDRRAGLVPPSWRASVAKSQLPSTCSRIEVAGTYVAATSTSFPSKQTPNPSAAPSPSPYPTACADASEVYVTFNERVSTAWGETIKVVGNVPALGNWDTSKAVTLSASGYKSNDPLWSITVPIKATGSAVQYKYIKVGTNGKITWESDPNRSITLQTASSAGKCAAQTVNDSWR

SEQ ID NO: 35 - protein, 639 aa, Aspergillus awamori
  SIGNAL 1..18
MSFRSLLALSGLVCSGLASVISKRATLDSWLSNEATVARTAILNNIGADGAWVSGADSGIVVASPSTDNPDYFYTWTRDSGLVIKTLVDLFRNGDTDLLSTIENYISSQAIVQGISNPSGDLSSGGLGEPKFNVDETAYTGSWGRPQRDGPALRATAMIGFRQWLLDNGYTSAATEIVWPLVRNDLSYVAQYWNQTGYDLWEEVNGSSFFTIAVQHRALVEGSAFATAVGSSCSWCDSQAPQILCYLQSFWTGEYILANFDSSRSGKDTNTLLGSIHTFDPEAGCDDSTFQPCSPRALANHKEVVDSFRSIYTLNDGLSDSEAVAVGRYPKDSYYNGNPWFLCTLAAAEQLYDALYQWDKQGSLEITDVSLDFFQALYSDAATGTYSSSSSTYSSIVDAVKTFADGFVSIVETHAASNGSLSEQYDKSDGDELSARDLTWSYAALLTANNRRNSVMPPSWGETSASSVPGTCAATSASGTYSSVTVTSSPSIVATGGTTTTATTTGFGGVTSTSKTTTTASKTSTTTSSTSCTTPTAVAVTFDLTATTTYGENIYLVGSISQLGDWDTSDGIALSADKYTSSNPLWYVTVTLPAGESFEYKFIRIESDDSVEWESDPNREYTVPQACGESTATVTDTWR

SEQ ID NO: 36 - protein, 639 aa, Aspergillus niger
  SIGNAL 1..18
MSFRSLLALSGLVCTGLANVISKRATWDSWLSNEATVARTAILNNIGADGAWVSGADSGIVVASPSTDNPDYFYTWTRDSGLVLKTLVDLFRNGDTSLLSTIENYISAQAIVQGISNPSGDLSSGAGLGEPKFNVDETAYTGSWGRPQRDGPALRATAMIGFGQWLLDNGYTSTATDIVWPLVRNDLSYVAQYWNQTGYDLWEVNGSSFFTIAVQHRALVEGSAFATAVGSSCSWCDSQAPEILCYLQSFWTGSFILANFDSSRSAKDANTLLLGSIHTFDPEAACDDSTFQPCSPRALANHKEVVDSFRSIYTLNDGLSDSEAVAVGRYPEDTYYNGNPWFLCTLAAAEQLYDALYQWDKQGSLEVTDVSLDFFKALYSDATGTYSSSSSTYSSIVDAVKTFADGFVSIVETHAASNGSMSEQYDKSDGEQLSARDLTWSYAALLTANNRRNVVPSASWGETSASSVPGTCAATSAIGTYSSVTVTSWPSIVATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 37 - protein, 515 aa, synthetic construct
  REGION 1..515: note = Mutated Saccharoymycopsis fibuligera glucoamylase
  SIGNAL 1..26
MIRLTVFLTAVFAAVASCVPVELDKRNTGHFQAYSGYTVNRSNFTQWIHEQPAVSWYYLLQNIDYPEGQFKSAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

(19) United States
(12) Patent Application Publication
(10) Pub. No.: US 2026/0103731 A1
(43) Pub. Date: Apr. 16, 2026

(54) PROCESS FOR DISPLACING AN EXOGENOUS ENZYME

(71) Applicant: DANSTAR FERMENT AG, Zug (CH)

(72) Inventors: Aaron ARGYROS, Lebanon, NH (US); Alexandra PANAITIU, Enfield, NH (US); Charles RICE, Plainfield, NH (US); Matthew B. RICHARDS, Lebanon, NH (US); Tera STOUGHTENGER, New Lisbon, WI (US); Kenneth BARRETT, San Diego, CA (US)

(73) Assignee: DANSTAR FERMENT AG, Zug (CH)

(21) Appl. No.: 19/416,347

(22) Filed: Dec. 11, 2025

Related U.S. Application Data
(63) Continuation of application No. 17/105,100, filed on Nov. 25, 2020, now Pat. No. 12,529,079.
(60) Provisional application No. 62/941,967, filed on Nov. 29, 2019.
(60) Provisional application No. 63/078,139, filed on Sep. 14, 2020.
(60) Provisional application No. 63/078,135, filed on Sep. 14, 2020.

(51) Int. Cl. C12P 7/06; C12N 1/18; C12N 9/60

(52) U.S. Cl. CPC C12P 7/06; C12N 1/18; C12N 9/60

(57) ABSTRACT

The present disclosure concerns a process for fermenting a biomass with a reduced dose of a purified exogenous enzyme (which can be, for example a purified exogenous glucoamylase). The process comprises contacting a biomass (which may comprise starch) with a recombinant yeast host cell. The recombinant yeast host cell has a genetic modification for expressing a heterologous polypeptide having starch or dextrin hydrolase activity (which may be, for example, from a glucoamylase). The nucleic acid molecule encoding the heterologous polypeptide having starch or dextrin hydrolase activity comprises allowing the secretion of the heterologous polypeptide.

### CROSS REFERENCE TO RELATED APPLICATIONS

This application is a continuation of U.S. patent application Ser. No. 17/105,100, filed Nov. 25, 2020, which claims the benefit of priority of U.S. Provisional Patent Application No. 63/078,139, filed Sep. 14, 2020, U.S. Provisional Patent Application No. 63/078,135, filed Sep. 14, 2020, and U.S. Provisional Patent Application No. 62/941,967, filed Nov. 29, 2019, all of which are hereby incorporated by reference in their entirety.

### SEQUENCE LISTING

The instant application contains a Sequence Listing which has been submitted in XML format and is hereby incorporated by reference in its entirety. Said XML copy, created on Dec. 9, 2025, is named NRORP0706USC1-Sequence-Listing.xml and is 69,214 bytes in size.

### TECHNOLOGICAL FIELD

The present disclosure relates to process for displacing, at least in part, an exogenous enzyme during a fermentation using a recombinant yeast host cell expressing a heterologous starch or dextrin hydrolase.

### BACKGROUND

Saccharomyces cerevisiae is the primary biocatalyst used in the commercial production of fuel ethanol. This organism is proficient in fermenting glucose to ethanol, often to concentrations greater than 20% v/v. However, S. cerevisiae lacks the ability to hydrolyze polysaccharides. Consequently, in addition to yeast, industrial ethanol production requires the exogenous addition of expensive enzymes to convert complex sugars to glucose. For example, in the United States, the primary source of fuel ethanol is corn starch. Regardless of the mashing process, corn starch fermentation by yeast benefits from the exogenous addition of both α-amylase and glucoamylase.

The fermentation processes employed in the corn ethanol industry can be broadly classified based on utilized substrate into liquefied corn mash and raw corn flour fermentations. In the mashing process, corn is both thermally and enzymatically liquefied prior to fermentation using α-amylase, which breaks down long chain starch polymers into smaller dextrins. The mash is then cooled and inoculated with S. cerevisiae. Concomitantly, the exogenous purified glucoamylase is added. Glucoamylases (GAs) break down the branched dextrin into glucose molecules that are utilizable by yeast. GAs primarily hydrolyze α-1,4-glycosidic linkages from non-reducing ends in starch chain (they are, hence, exo-acting enzymes), while α-amylases can also hydrolyze α-1,6-glicosidic linkages from the inner starch chains (and are, therefore, endo-acting enzymes). The availability of a robust, ethanol-tolerant yeast strain is required to ferment the hydrolyzed starch into the desired final product, ethanol.

In dry mill fuel ethanol production from grain, incoming starch-rich grain (typically corn, but also milo or wheat) can be reduced in size in a hammer or roller mill to create a flour, which can be blended with water, recycled thin stillage (“backset”), and exogenous alpha amylase enzymes to create a slurry. This slurry can be heated with steam and held at elevated temperature during a mashing or cooking process to gelatinize the starch and allow hydrolysis of the starch by the alpha-amylase enzymes to convert the starch to a range of soluble and insoluble dextrins. Having gone through this cooking process, the slurry (now referred to as “liquefact” or “mash”) can be cooled to fermentation temperatures and used to fill fermentors for the production of fuel ethanol by yeast fermentation.

Yeast (especially Saccharomyces cerevisiae) can be added to fermentation after having been allowed to grow on a portion of the liquefact in a separate vessel for several generations in order to increase the total number of yeast cells added to fermentation and to acclimate the yeast to the liquefact in a process referred to as “mash propagation”; however, yeasts can also be added directly to fermentation without this additional growth step in a process variation known as “direct pitch.” Nitrogen for growth of the yeast can be provided to fermentation through amino acids in the liquefact (the concentration of which is sometimes increased through addition of exogenous protease enzymes in fermentation), the addition of urea to fermentation, and/or the addition of ammonia directly to fermentation or to the mash during the cooking process. The state of the art is that exogenous glucoamylase (GA) enzyme cocktails can be added to fermentation to hydrolyze the starch dextrins in the liquefact to glucose, allowing for uptake and fermentation of the glucose to ethanol by the yeast. This process is typically referred to as simultaneous saccharification and fermentation (SSF). Combination of these components in fermentation results in a fermenting slurry referred to as “beer.” Exogenous GA enzyme cocktails are purchased for this purpose by fuel ethanol producers at substantial cost, generally as concentrated products in a stabilized formulation having undergone some level of filtration or purification from the enzyme production culture. For production facilities using mash propagation to increase the total number of yeast cells added to fermentation, a small amount of exogenous GA enzyme cocktail can be added to the mash in propagation in order to release glucose to provide for growth of the yeast cells; the volume of exogenous GA enzyme cocktail added to each mash propagation batch is typically less than 3 gallons, or typically less than 2.5×10⁻⁴ v/v (volume of exogenous GA enzyme cocktail per total volume of mash propagation batch).

It would be highly desirable to be provided with improved and/or more efficient yeast strains which reduces or precludes the need for highly expensive enzyme purification and formulation, thus significantly reducing overall production costs.

### BRIEF SUMMARY

The present disclosure provides recombinant yeast host cells which expresses starch digesting glucoamylases that can be used in saccharification and fermentation of a biomass. The present disclosure concerns recombinant yeast host cells expressing a heterologous starch digesting glucoamylase by introducing a heterologous nucleic acid molecule encoding for the glucoamylase enzyme as well as a signal sequence allowing the secretion of the glucoamylase. The present disclosure also provides a process for making a fermentation product from a biomass while reducing the dose of an exogenous enzyme to complete the fermentation.

According to a first aspect, the present disclosure provides a recombinant yeast host cell for saccharification and fermentation of a biomass, the recombinant yeast host cell having a heterologous nucleic acid molecule encoding a heterologous polypeptide having glucoamylase activity. The heterologous nucleic acid molecule comprises a first polynucleotide encoding a heterologous signal sequence wherein the heterologous signal sequence has the amino acid sequence of SEQ ID NO: 5, is a variant of the amino acid sequence of SEQ ID NO: 5 having signal sequence activity, or is a fragment of the amino acid sequence of SEQ ID NO: 5 having signal sequence activity. The heterologous nucleic acid also comprises a second polynucleotide encoding the heterologous polypeptide having glucoamylase activity, wherein the polypeptide having glucoamylase activity has the amino acid sequence of SEQ ID NO: 3, 13 or 27 to 36, is a variant of the amino acid sequence of SEQ ID NO: 3, 13 or 27 to 36 having glucoamylase activity, or is a fragment of the amino acid sequence of SEQ ID NO: 3 or 13 or 27 to 36 having glucoamylase activity. In the recombinant yeast host cell of the present disclosure, the first polynucleotide molecule is operatively associated with the second polynucleotide molecule. In an embodiment, the heterologous nucleic acid molecules encodes the heterologous polypeptide having the amino acid sequence of SEQ ID NO: 1 or 11, a variant of the amino acid sequence of SEQ ID NO: 1 or 11 having glucoamylase activity, or a fragment of the amino acid sequence of SEQ ID NO: 1 or 11 having glucoamylase activity. In yet another embodiment, the heterologous nucleic acid molecule further comprises a third polynucleotide comprising a heterologous promoter operatively associated with the first polynucleotide and the second polynucleotide allowing the expression of the heterologous polypeptide having glucoamylase activity. In an embodiment, the heterologous promoter is capable of allowing the expression of the heterologous polypeptide having glucoamylase activity during propagation. In an embodiment, the heterologous polypeptide having glucoamylase activity is a secreted polypeptide. In another embodiment, the heterologous polypeptide having glucoamylase activity is a membrane-associated polypeptide, such as, for example, a tethered polypeptide. In an embodiment, the recombinant yeast host cell comprises a further heterologous nucleic acid molecule encoding a heterologous alpha-amylase and/or a heterologous glucoamylase. In some specific embodiments, the heterologous alpha-amylase has the amino acid sequence of any one of SEQ ID NO: 17 to 26, is a variant of the amino acid sequence of any one of SEQ ID NO: 17 to 26 having alpha-amylase activity or is a fragment of the amino acid sequence of any one of SEQ ID NO: 17 to 26 having alpha-amylase activity. In some specific embodiments, the heterologous glucoamylase has the amino acid sequence of any one of SEQ ID NO: 27 to 36, a variant of the amino acid sequence of any one of SEQ ID NO: 27 to 36 having glucoamylase activity or a fragment of the amino acid sequence of any one of SEQ ID NO: 27 to 36 having glucoamylase activity. In an embodiment, the recombinant yeast host cell is from the genus Saccharomyces, such as, for example, from the species Saccharomyces cerevisiae.

According to a second aspect, the present disclosure provides a composition comprising the recombinant yeast host cell described herein and starch.

According to a third aspect, the present disclosure provides a process for saccharification and fermentation of a biomass into a fermentation product, the process comprises contacting the biomass with the recombinant yeast host cell defined herein or the composition defined herein, under a condition that allows the conversion of at least a part of the biomass into the fermentation product (in some embodiments during a fermentation). In an embodiment, the biomass is derived from or comprises corn, potato, cassava, rice, wheat, cellulosic material, milo (grain sorghum) or buckwheat. In another embodiment, the biomass is derived from or comprises corn. In still another embodiment, the biomass comprises or is corn mash. In an embodiment, the fermentation product is ethanol. In another embodiment, the conversion/fermentation is conducted in the presence of a stressor. In yet a further embodiment, the stressor is low pH (such as, for example, a pH of 5.0 or lower or a pH of 4.0 or lower). In still a further embodiment, the stressor is an elevated temperature. In some embodiments, the process avoids including a purified exogenous enzyme (e.g., achieves 100% enzyme displacement). In some embodiments, the purified exogenous enzyme is a glucoamylase.

According to a fourth aspect, the present disclosure provides a process for fermenting a biomass into a fermentation product. The process comprises contacting the biomass with a recombinant yeast host cell and a reduced dose of a purified exogenous enzyme, under a condition that allows the conversion of at least a part of the biomass into the fermentation product. The recombinant yeast host cell has a heterologous nucleic acid molecule encoding a heterologous polypeptide having starch or dextrin hydrolase activity, wherein the heterologous nucleic acid molecule comprises a first polynucleotide encoding a heterologous signal sequence; and a second polynucleotide encoding a heterologous polypeptide having starch or dextrin hydrolase activity. The first polynucleotide molecule is operatively associated with the second polynucleotide molecule. The exogenous enzyme is a starch or dextrin hydrolase. The reduced dose of the purified exogenous enzyme is lower than a control dose necessary for a control yeast host cell lacking the ability to hydrolyze starch or dextrin to complete a corresponding control fermentation. In an embodiment, the control yeast host cell lacks the heterologous nucleic acid molecule. In still another embodiment, the heterologous polypeptide having starch or dextrin hydrolase activity is an heterologous polypeptide having glucoamylase activity. In yet another embodiment, the polypeptide having glucoamylase activity has the amino acid sequence of SEQ ID NO: 3, 13 or 27 to 36, is a variant of the amino acid sequence of SEQ ID NO: 3, 13 or 27 to 36 having glucoamylase activity, or is a fragment of the amino acid sequence of SEQ ID NO: 3, 13 or 27 to 36 having glucoamylase activity. In still a further embodiment, the heterologous signal sequence has the amino acid sequence of SEQ ID NO: 5, is a variant of the amino acid sequence of SEQ ID NO: 5 having signal sequence activity, or is a fragment of the amino acid sequence of SEQ ID NO: 5 having signal sequence activity. In yet another embodiment, the heterologous nucleic acid molecule encodes the heterologous polypeptide having the amino acid sequence of SEQ ID NO: 1 or 11, a variant of the amino acid sequence of SEQ ID NO: 1 or 11 having glucoamylase activity, or a fragment of the amino acid sequence of SEQ ID NO: 1 or 11 having glucoamylase activity. In still another embodiment, the heterologous nucleic acid molecule further comprises a third polynucleotide comprising a heterologous promoter operatively associated with the first polynucleotide and the second polynucleotide allowing the expression of the heterologous polypeptide having starch or hydrolase activity. In an embodiment, the heterologous promoter is capable of allowing the expression of the heterologous polypeptide having starch or dextrin hydrolase activity during propagation. In still a further embodiment, the heterologous polypeptide having starch or dextrin activity is a secreted polypeptide. In yet another embodiment, the heterologous polypeptide having starch or dextrin hydrolase activity is a membrane-associated polypeptide. In yet another embodiment, the membrane-associated polypeptide is a tethered polypeptide. In an embodiment, the recombinant yeast host cell is from the genus Saccharomyces and, in a further embodiment, from the species Saccharomyces cerevisiae. In yet another embodiment, the biomass comprises starch or a starch derivative. In still a further embodiment, the biomass is derived from or comprises corn, potato, cassava, rice, wheat, lignocellulosic material or buckwheat. In yet another embodiment, the biomass is derived from or comprises corn. In specific embodiments the biomass comprises or is corn mash. In an embodiment, the fermentation product is ethanol. In still another embodiment, the control dose allows achieving a fermentation yield of at least 0.415%, w/v per w/w of biomass. In a further embodiment, the reduced dose allows achieving a fermentation yield of at least 0.440%, w/v per w/w of biomass. In still another embodiment, the reduced dose of the purified exogenous enzyme is lower by at least 50%, 55%, 60%, 61%, 62%, 63%, 64%, 65%, 66%, 67%, 68%, 69%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% when compared to the control dose. In a specific embodiment, the process excludes including a purified exogenous enzyme in the biomass prior to or during fermentation. In an embodiment, the reduced dose of the exogenous enzymes is reduced to zero. In still another embodiment, the fermentation yield is equal to or higher than the control fermentation yield of the control fermentation. In yet another embodiment, the fermentation yield is substantially similar to the control fermentation yield of the control fermentation. In some embodiments, the exogenous enzyme is a glucoamylase.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 illustrates the data associated with a corn mash fermentation (analyzed 23 hours into the fermentation). Bars represent midpoint ethanol titers in fermentation (plotted on the left y axis, in g/L). Lozenges (♦) denote glucose titers and triangles (▴) represent yeast-produced (YP) glycerol titers (right y axis, all in g/L). Results are shown for strains M2390, M17199, M15621, M23176 and M23177 (see Table 1 for a description of the strains). The amount (% of control dose) of exogenous glucoamylase used is listed under each bar.

FIG. 2 illustrates the data associated with a corn mash fermentation (analyzed 48 hours into the fermentation). Bars represent endpoint ethanol titers (plotted on the left y axis, in g/L). Lozenges (♦) denote glucose titers and triangles (▴) represent yeast-produced (YP) glycerol titers (right y axis, all in g/L). Results are shown for strains M2390, M17199, M15621, M23176 and M23177 (see Table 1 for a description of the strains). The amount (% of control dose) of exogenous glucoamylase used is listed under each bar.

FIG. 3 illustrates the data associated with a permissive corn mash fermentation in the presence or absence of exogenous glucoamylase (analyzed 48 hours into the fermentation). Bars represent endpoint ethanol titers (plotted on the left y axis, in g/L). Lozenges (♦) denote glucose titers and triangles (▴) represent yeast-produced (YP) glycerol titers (right y axis, all in g/L). Results are shown for strains M2390, M17199, M15621 and M23177. The amount (% of control dose) of exogenous glucoamylase used is listed under each bar.

FIG. 4 illustrates the data associated with a non-permissive (lactic acid treatment) corn mash fermentation (analyzed 48 hours into the fermentation). Bars represent endpoint ethanol titers (plotted on the left y axis, in g/L). Lozenges (♦) denote glucose titers and triangles (▴) represent yeast-produced (YP) glycerol titers (right y axis, all in g/L). Results are shown for strains M2390, M17199, M15621 and M23177 in the presence or absence of exogenous glucoamylase. The amount (% of control dose) of exogenous glucoamylase used is listed under each bar.

FIG. 5 illustrates the data associated with a non-permissive (heat treatment) corn mash fermentation (analyzed 48 hours into the fermentation). Bars represent endpoint ethanol titers (plotted on the left y axis, in g/L). Lozenges (♦) denote glucose titers and triangles (▴) represent yeast-produced (YP) glycerol titers (right y axis, all in g/L). Results are shown for strains M2390, M17199, M15621 and M23177. The amount (% of control dose) of exogenous glucoamylase used is listed under each bar.

FIG. 6 illustrates the average batch ethanol yield at 60 h fermentation duration (ethanol titer normalized to total solids of slurry in cooking process per fermentation batch, results are shown as % w/v per % w/w). The conventional (not genetically modified) yeast (Phase A) was used with 100 gal of exogenous glucoamylase cocktail in fermentation, strain M15419 (Phases B and C) was used with 51 gal of exogenous glucoamylase cocktail in fermentation, and strain M23541 (Phases C to K) was used with between zero exogenous glucoamylase cocktail in mash propagation and fermentation and 17 gal of exogenous glucoamylase cocktail in fermentation. As the total final fermentation durations varied between yeast strain types and conditions, data is presented here for all conditions after 60 h of fermentation (60 h following the transfer of mash propagation to fermentation). Error bars indicate ±1 standard deviation from the average.

FIG. 7 illustrates the average batch ethanol yield (ethanol titer normalized to total solids of slurry in cooking process per fermentation batch, results are shown as % w/v per % w/w) at the end of fermentation (fermentation drop). The conventional (not genetically modified) yeast (Phase A) was used with 100 gal of exogenous glucoamylase cocktail in fermentation, strain M15419 (Phases B and C) was used with 51 gal of exogenous glucoamylase cocktail in fermentation, and strain M23541 (Phase D to K) was used with between zero exogenous glucoamylase cocktail in mash propagation and fermentation and 17 gal of exogenous glucoamylase cocktail in fermentation. Error bars indicate ±1 standard deviation from the average.

FIG. 8 illustrates the average batch ethanol yield (ethanol titer normalized to total solids of slurry in cooking process per fermentation batch, results are shown as % w/v per % w/w) of strain M23541 over the course of fermentations. The conventional (not genetically modified) yeast (Phase A) was used with 100 gal of exogenous glucoamylase cocktail in fermentation, strain M15419 (Phases B and C) was used with 51 gal of exogenous glucoamylase cocktail in fermentation, and strain M23541 (Phases D to K) was used with between zero exogenous glucoamylase cocktail in mash propagation and fermentation and 17 gal of exogenous glucoamylase cocktail in fermentation. Error bars indicate ±1 standard deviation from the average for the conventional (not genetically modified) yeast. Letter indicated in legend represents testing phase, and percentage indicated in legend represents percentage displacement of exogenous GA enzyme cocktail.

FIG. 9 illustrates the data associated with permissive fermentations analyzed 24 hours and 52 hours into the fermentation. Bars represent endpoint ethanol titers (plotted on the left y axis, in g/L). Results are shown for strains Ethanol Red (e.g., active dry yeast or ADY), M23177, M24926 and M23541 in function of the amount (% of dose) of exogenous glycoamylase used (both listed under each bar).

FIG. 10 illustrates the ethanol titer data of FIG. 9 obtained at 52 hours, but plotted as relative ethanol yield compared to Ethanol Red (ADY). For each strain (M23177♦, M24926• and M23541▪) and each enzyme inclusion, the drop ethanol titer is plotted as a ratio to the drop ethanol titer of Ethanol Red (ADY) with 100% enzyme inclusion (y axis). The exogenous enzyme inclusion (% of dose) for each condition is reported as a percentage from the dose used with ADY and is indicated on the x axis.

### DETAILED DESCRIPTION

As shown herein, yeast strains genetically engineered to secrete a starch or dextrin hydrolase (such as for example a glucoamylase) can make a fermentation product (such as ethanol) using reduced levels (e.g., a reduced dose) of exogenous enzyme (which may be present in the form of a cocktail) to complete the fermentation.

In dry mill fuel ethanol production facilities, corn milling, the mashing or cooking process, and finished fermentation beer distillation and product recovery unit operations are carried out continuously. In facilities utilizing batch fermentation, the liquefact mash is fermented in batches (or, more specifically, fed-batches) in the otherwise continuous ethanol production process. Facilities utilizing batch fermentation have multiple fermentation vessels (“fermentors”, typically between 4 and 7 vessels for facilities as originally built) operated in parallel. The fermentors are operated in a cyclical mode such that at any point in time one fermentor is being filled with liquefact, the finished fermentation beer from another fermentor is being transferred to the holding vessel prior to distillation (the “beer well”) and the emptied fermentor cleaned, and the remaining fermentors are in various stages of fermentation (measured by duration following the start of fill of the fermentor with liquefact). The duration of one cycle of fermentors is set by the total number of fermentors in use at the production facility, the working volume of the fermentors (the total volume to which fermentors are filled with liquefact, transfer of mash propagation or direct pitch yeast addition, exogenous GA enzyme cocktail and other added fermentation enzymes, nitrogen source addition, and any other fermentation ingredients), and the volumetric flow rate of liquefact discharged from the mashing process at the facility. One fermentation batch occurs during the period between when liquefact begins to be added to a fermentor during a cycle and when the finished fermentation beer begins to be transferred to the beer well (the time referred to as fermentation “drop” or the “end of fermentation”) during the same cycle. The maximum duration of a fermentation batch at a production facility is one cycle duration minus the time required to empty and clean the fermentor following the batch in order to prepare for the next cycle.

The present disclosure also provides recombinant yeast host cells that can be used under conditions of saccharification and fermentation of a biomass. Glucoamylase are usually secreted by the cell expressing a gene encoding same. Most glucoamylase sequences include a signal sequence which enables or facilitates the secretion of the enzyme. In the present disclosure, it has been recognized that using a signal sequence from the Saccharomyces cerevisiae alpha-mating factor 1 (e.g., having the amino acid sequence of SEQ ID NO: 5, a variant thereof or a fragment thereof) allows the displacement, at least in part, of exogenous (purified) glucoamylase during the fermentation process. In some additional embodiments, using a signal sequence from the Saccharomyces cerevisiae alpha-mating factor 1 (e.g., having the amino acid sequence of SEQ ID NO: 5, a variant thereof or a fragment thereof) increased the robustness of the recombinant yeast host cell expressing same.

### Recombinant Yeast Host Cell

The heterologous polypeptides having starch or dextrin hydrolase activity (such as, for example, glucoamylases) are expressed in a recombinant yeast host cell. As used in the context of the present disclosure, a heterologous polypeptide having starch or dextrin hydrolase activity is a polypeptide capable of cleaving a starch and/or a dextrin molecule into smaller fragments. As such, the recombinant yeast host cell of the present disclosure includes at least one genetic modification. In the context of the present disclosure, when recombinant yeast cell is qualified has “having a genetic modification” or as being “genetically engineered”, it is understood to mean that it has been manipulated to either add at least one or more heterologous or exogenous nucleic acid residue and/or remove at least one endogenous (or native) nucleic acid residue. The genetic manipulation(s) did not occur in nature and is the results of in vitro manipulations of the recombinant host cell. When the genetic modification is the addition of a heterologous nucleic acid molecule, such addition can be made once or multiple times at the same or different integration sites. When the genetic modification is the modification of an endogenous nucleic acid molecule, it can be made in one or both copies of the targeted gene/non-coding region. In a specific embodiment, the recombinant yeast host cell having the genetic modification has a heterologous nucleic acid molecule encoding a heterologous polypeptide having glucoamylase activity.

When expressed in a recombinant yeast host cell, the heterologous polypeptide (such as those having glucoamylase activity, e.g. a glucoamylase) described herein are encoded on one or more heterologous nucleic acid molecule. In some embodiments, heterologous polypeptide described herein can be encoded on one heterologous nucleic acid molecule, two heterologous nucleic acid molecules or copies, three heterologous nucleic acid molecules or copies, four heterologous nucleic acid molecules or copies, five heterologous nucleic acid molecules or copies, six heterologous nucleic acid molecules or copies, seven heterologous nucleic acid molecules or copies, or eight or more heterologous nucleic acid molecules or copies. The term “heterologous” when used in reference to a nucleic acid molecule (such as a promoter or a coding sequence) refers to a nucleic acid molecule that is not natively found in the recombinant yeast host cell. “Heterologous” also includes a native coding region, or portion thereof, that was removed from the organism (which can, in some embodiments, be a source organism) and subsequently reintroduced into the organism in a form that is different from the corresponding native gene, e.g., not in its natural location in the organism's genome. The heterologous nucleic acid molecule is purposively introduced into the recombinant yeast host cell. The term “heterologous” as used herein also refers to an element (nucleic acid or polypeptide) that is derived from a source other than the endogenous source. Thus, for example, a heterologous element could be derived from a different strain of host cell, or from an organism of a different taxonomic group (e.g., different kingdom, phylum, class, order, family genus, or species, or any subgroup within one of these classifications).

When a heterologous nucleic acid molecule is present in the recombinant yeast host cell, it can be integrated in the host cell's chromosome. The term “integrated” as used herein refers to genetic elements that are placed, through molecular biology techniques, into the chromosome(s) of the recombinant yeast host cell. For example, genetic elements can be placed into the chromosome(s) of the host cell as opposed to in a vector such as a plasmid carried by the host cell. Methods for integrating genetic elements into the chromosome(s) of a host cell are well known in the art and include homologous recombination. The heterologous nucleic acid molecule can be present in one or more copies in the yeast host cell's chromosome(s). Alternatively, the heterologous nucleic acid molecule can be independently replicating from the yeast's chromosome. In such embodiment, the nucleic acid molecule can be stable and self-replicating.

In the context of the present disclosure, the yeast host cell can be a recombinant yeast host cell. Suitable yeast host cells can be, for example, from the genus Saccharomyces, Kluyveromyces, Arxula, Debaryomyces, Candida, Pichia, Phaffia, Schizosaccharomyces, Hansenula, Kloeckera, Schwanniomyces or Yarrowia. Suitable yeast species can include, for example, Saccharomyces cerevisiae, Saccharomyces bulderi, Saccharomyces barnetti, Saccharomyces exiguus, Saccharomyces uvarum, Saccharomyces diastaticus, Kluyveromyces lactis, Kluyveromyces marxianus or Kluyveromyces fragilis. In some embodiments, the yeast is selected from the group consisting of Saccharomyces cerevisiae, Schizzosaccharomyces pombe, Candida albicans, Pichia pastoris, Pichia stipitis (Komagatella phaffi), Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe and Schwanniomyces occidentalis. In one particular embodiment, the yeast is Saccharomyces cerevisiae. In some embodiments, the host cell can be an oleaginous yeast cell. For example, the oleaginous yeast host cell can be from the genus Blakeslea, Candida, Cryptococcus, Cunninghamella, Lipomyces, Mortierella, Mucor, Phycomyces, Pythium, Rhodosporidum, Rhodotorula, Trichosporon or Yarrowia. In some alternative embodiments, the host cell can be an oleaginous microalgae host cell (e.g., for example, from the genus Thraustochytrium or Schizochytrium). In an embodiment, the recombinant yeast host cell is from the genus Saccharomyces and, in some additional embodiments, from the species Saccharomyces cerevisiae.

In some embodiments, the nucleic acid molecules encoding the heterologous polypeptides, fragments or variants that can be introduced into the recombinant yeast host cells are codon-optimized with respect to the intended recipient recombinant yeast host cell. As used herein the term “codon-optimized coding region” means a nucleic acid coding region that has been adapted for expression in the cells of a given organism by replacing at least one, or more than one, codons with one or more codons that are more frequently used in the genes of that organism. In general, highly expressed genes in an organism are biased towards codons that are recognized by the most abundant tRNA species in that organism. One measure of this bias is the “codon adaptation index” or “CAI,” which measures the extent to which the codons used to encode each amino acid in a particular gene are those which occur most frequently in a reference set of highly expressed genes from an organism. The CAI of codon optimized heterologous nucleic acid molecule described herein corresponds to between about 0.8 and 1.0, between about 0.8 and 0.9, or about 1.0.

The heterologous nucleic acid molecules of the present disclosure can comprise a coding region for the heterologous polypeptide. A DNA or RNA “coding region” is a DNA or RNA molecule which is transcribed and/or translated into a polypeptide in a cell in vitro or in vivo when placed under the control of appropriate regulatory sequences. “Suitable regulatory regions” refer to nucleic acid regions located upstream (5′ non-coding sequences), within, or downstream (3′ non-coding sequences) of a coding region, and which influence the transcription, RNA processing or stability, or translation of the associated coding region. Regulatory regions may include promoters, translation leader sequences, RNA processing site, effector binding site and stem-loop structure. The boundaries of the coding region are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell, a polyadenylation signal and transcription termination sequence will usually be located 3′ to the coding region. In an embodiment, the coding region can be referred to as an open reading frame. “Open reading frame” is abbreviated ORF and means a length of nucleic acid, either DNA, cDNA or RNA, that comprises a translation start signal or initiation codon, such as an ATG or AUG, and a termination codon and can be potentially translated into a polypeptide sequence.

The nucleic acid molecules described herein can comprise transcriptional and/or translational control regions. “Transcriptional and translational control regions” are DNA regulatory regions, such as promoters, enhancers, terminators, and the like, that provide for the expression of a coding region in a host cell. In eukaryotic cells, polyadenylation signals are control regions.

The heterologous nucleic acid molecule can be introduced in the host cell using a vector. A “vector,” e.g., a “plasmid”, “cosmid” or “artificial chromosome” (such as, for example, a yeast artificial chromosome) refers to an extra chromosomal element and is usually in the form of a circular double-stranded DNA molecule. Such vectors may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

### Heterologous Polypeptides Having Starch or Dextrin Hydrolase Activity and Nucleic Acid Molecules Encoding Same

The heterologous nucleic acid molecule present in the recombinant yeast host cell includes a heterologous polynucleotide encoding a signal sequence. As it is known in the art, a signal sequence corresponds to a short stretch of amino acid residues (usually no longer than 50 contiguous amino acids and usually located at the amino terminus of the polypeptide) which are capable of guiding the remainder of the polypeptide for secretion. The signal sequence is usually cleaved upon the secretion of the polypeptide and thus is not necessarily involved with the enzymatic activity of the secreted polypeptide (e.g., glucoamylase activity in the present disclosure). In embodiments, the signal sequence encoded by the heterologous nucleic acid molecule (which can be associated with the heterologous polypeptide having glucoamylase activity) can have the amino acid sequence of SEQ ID NO: 5 or of the section spanning residues 1 to 21 of SEQ ID NO: 17, 1 to 21 of SEQ ID NO: 18, 1 to 23 of SEQ ID NO: 19, 1 to 19 of SEQ ID NO: 20, 1 to 25 of SEQ ID NO: 21, 1 to 22 of SEQ ID NO: 22, 1 to 29 of SEQ ID NO: 23, 1 to 16 of SEQ ID NO: 24, 1 to 23 of SEQ ID NO: 25, 1 to 21 of SEQ ID NO: 26, 1 to 17 of SEQ ID NO: 27, 1 to 20 of SEQ ID NO: 28, 1 to 22 of SEQ ID NO: 29, 1 to 18 of SEQ ID NO: 30, 1 to 25 of SEQ ID NO: 31, 1 to 19 of SEQ ID NO: 32, 1 to 18 of SEQ ID NO: 33, 1 to 19 of SEQ ID NO: 34, 1 to 18 of SEQ ID NO: 35, 1 to 18 of SEQ ID NO: 36 as well as variants and fragments thereof. In embodiments in which the heterologous polypeptide having glucoamylase activity has the amino acid sequence of SEQ ID NO: 3 or 13, the signal sequence encoded by the heterologous nucleic acid molecule (which can be associated with the heterologous polypeptide having glucoamylase activity) can have the amino acid sequence of SEQ ID NO: 5, a variant thereof or a fragment thereof.

The first polynucleotide can encode a signal sequence, a variant of a signal sequence having signal sequence activity or a fragment of a signal sequence having signal sequence activity. A variant signal sequence comprises at least one amino acid difference when compared to the amino acid sequence of the native or wild-type signal sequence and exhibits a biological activity substantially similar to the native (wild-type) signal sequence (e.g., the ability to guide the heterologous polypeptide having glucoamylase activity for secretion). The signal sequence “variants” have at least 50%, 55%, 60%, 65%, 70%, 75%, 80%, 85%, 90%, 95%, 96%, 97%, 98% or 99% of the biological activity when compared to the wild-type signal sequence described herein. The signal sequence “variants” have at least 50%, 55%, 60%, 65%, 70%, 75%, 80%, 85%, 90%, 95%, 96%, 97%, 98% or 99% identity to the wild-type signal sequence described herein. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variant signal sequence described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group. A “variant” of the wild-type signal sequence can be a conservative variant or an allelic variant. As used herein, a conservative variant refers to alterations in the amino acid sequence that do not adversely affect the biological functions of the signal sequence. A substitution, insertion or deletion is said to adversely affect the signal sequence when the altered sequence prevents or disrupts a biological function associated with the signal sequence. For example, the overall charge, structure or hydrophobic-hydrophilic properties of the signal sequence can be altered without adversely affecting a biological activity. Accordingly, the amino acid sequence can be altered, for example to render the signal sequence more hydrophobic or hydrophilic, without adversely affecting the biological activities of the signal sequence.

The signal sequence can be a fragment of the signal sequence or a fragment of a variant signal sequence. A signal sequence fragment comprises at least one less amino acid residue when compared to the amino acid sequence of the full length signal sequence or variant possesses and still possess a biological activity substantially similar to the native full-length signal sequence or variant. The signal sequence “fragments” have at least 50%, 55%, 60%, 65%, 70%, 75%, 80%, 85%, 90%, 95%, 96%, 97%, 98% or 99% of the biological activity when compared to the full-length signal sequence or variants described herein. Signal sequence “fragments” have at least at least 2, 3, 4, 5, 6, 7, 8, 9, 10 or more consecutive amino acids of the full-length signal sequence or variants described herein. The signal sequence “fragments” can have at least 50%, 55%, 60%, 65%, 70%, 75%, 80%, 85%, 90%, 95%, 96%, 97%, 98% or 99% identity to the full-length signal sequence or variants described herein.

The heterologous nucleic acid molecule of the present disclosure also includes a second polynucleotide encoding the heterologous polypeptide having starch or dextrin hydrolase activity (such as an heterologous polypeptide having glucoamylase activity). In the context of the present disclosure, the first and second polynucleotides are in frame and operatively associated so as to encode a single polypeptide (which is intended to be cleaved so as to release the polypeptide having glucoamylase activity upon the secretion of the single polypeptide). In the heterologous nucleic acid molecule, the first polynucleotide is located upstream (5′) with respect to the second polynucleotide. Otherwise stated, the second polynucleotide is located downstream (3′) with respect to the first polynucleotide in the heterologous nucleic acid sequence.

As indicated above, the recombinant yeast host cell can bear a genetic modification for expressing at least one heterologous polypeptide having starch or dextrin hydrolase activity (such as a heterologous polypeptide having glucoamylase activity). In some embodiments, the recombinant yeast host cell is obtained by introducing one or more heterologous nucleic acid molecule encoding one or more of the heterologous polypeptide in the recombinant yeast host cell. In some embodiments, the genetic modification(s) in the recombinant yeast host cell of the present disclosure comprise or consist essentially of or consist of expressing a heterologous polypeptide having starch or dextrin hydrolase activity. In the context of the present disclosure, the expression “the genetic modification in the recombinant yeast host consist essentially of a genetic modification for expressing a heterologous polypeptide having starch digesting glucoamylase activity” refers to the fact that the recombinant yeast host cell only includes this genetic modification to modulate the expression of a polypeptide having starch digesting glucoamylase activity levels but can nevertheless include other genetic modifications which are unrelated to the expression of a glucoamylase (native or heterologous).

As indicated above, the heterologous polypeptide is a polypeptide having starch or dextrin digesting activity. As used herein, a polypeptide having glucoamylase activity refers to a polypeptide having the ability to hydrolyze starch (which can have been heat-treated) directly to glucose. For example, a polypeptide having glucoamylase activity may comprise a catalytic domain and a starch binding domain. The catalytic domain and the starch binding domain may be connected by a connecting loop or linker. In some alternative embodiments, the polypeptides having glucoamylase activity can be derived from a fungus, for example, from the genus Rasamsonia (sometimes known or referred to as Talaromyces) and, in some instances, from the species Rasamsonia emersonii (sometimes known or referred to as Talaromyces emersonii). In some specific embodiments, the heterologous polypeptide having starch digesting glucoamylase activity can have the amino acid sequence of SEQ ID NO: 3 (which refers to Uniprot Q9C1V4), be a variant of the amino acid sequence of SEQ ID NO: 3 (having glucoamylase activity) or be a fragment of the amino acid sequence of SEQ ID NO: 3 (having glucoamylase activity). In yet another specific embodiment, the heterologous nucleic acid molecule can comprise the nucleic acid sequence of SEQ ID NO: 4, be a variant of the nucleic acid sequence of SEQ ID NO: 4 (encoding a glucoamylase) or be a fragment of the nucleic acid sequence of SEQ ID NO: 4 (encoding a glucoamylase). In another specific embodiment, the heterologous polypeptide having glucoamylase activity can have the amino acid sequence of SEQ ID NO: 1, be a variant of the amino acid sequence of SEQ ID NO: 1 (having glucoamylase activity) or be a fragment of the amino acid sequence of SEQ ID NO: 1 (having glucoamylase activity). In yet another specific embodiment, the heterologous nucleic acid molecule can comprise the nucleic acid sequence of SEQ ID NO: 2, be a variant of the nucleic acid sequence of SEQ ID NO: 2 (encoding a glucoamylase) or be a fragment of the nucleic acid sequence of SEQ ID NO: 2 (encoding a glucoamylase). In some specific embodiments, the heterologous polypeptide having starch digesting glucoamylase activity can have the amino acid sequence of SEQ ID NO: 13 (which refers to Uniprot A0A0F4YWQ6), be a variant of the amino acid sequence of SEQ ID NO: 13 (having glucoamylase activity) or be a fragment of the amino acid sequence of SEQ ID NO: 13 (having glucoamylase activity). In yet another specific embodiment, the heterologous nucleic acid molecule can comprise the nucleic acid sequence of SEQ ID NO: 14, be a variant of the nucleic acid sequence of SEQ ID NO: 14 (encoding a glucoamylase) or be a fragment of the nucleic acid sequence of SEQ ID NO: 14 (encoding a glucoamylase). In another specific embodiment, the heterologous polypeptide having glucoamylase activity can have the amino acid sequence of SEQ ID NO: 11, be a variant of the amino acid sequence of SEQ ID NO: 11 (having glucoamylase activity) or be a fragment of the amino acid sequence of SEQ ID NO: 11 (having glucoamylase activity). In yet another specific embodiment, the heterologous nucleic acid molecule can comprise the nucleic acid sequence of SEQ ID NO: 12, be a variant of the nucleic acid sequence of SEQ ID NO: 12 (encoding a glucoamylase) or be a fragment of the nucleic acid sequence of SEQ ID NO: 12 (encoding a glucoamylase).

In some further embodiments, the recombinant yeast host cell can include a further genetic modification (which can be the introduction of a further heterologous nucleic acid molecule) for expressing a further heterologous glucoamylase (e.g., different from the R. emersonii glucoamylase described above). For example, the further heterologous glucoamylase can be from a Gloeophyllum sp., such as, for example, from Gloeophyllum trabeum. In an embodiment, the further heterologous glucoamylase corresponds to Uniprot S7Q4V9 or GenBank Accession Number_007866834. In another embodiment, the further heterologous glucoamylase can have the amino acid sequence of SEQ ID NO: 27, be a variant of the amino acid sequence of SEQ ID NO: 27 having glucoamylase activity or be a fragment of the amino acid sequence of SEQ ID NO: 27 having glucoamylase activity (which can, in an embodiment, correspond to a fragment of the amino acid sequence of SEQ ID NO: 27 lacking its signal sequence, e.g., for example between residues 18 to 576 of SEQ ID NO: 27). For example, the further heterologous glucoamylase can be from a Trichoderma sp., such as, for example, from Trichoderma reesii. In an embodiment, the further heterologous glucoamylase corresponds to Uniprot GOR866 or GenBank Accession Number_XP_006960925. In another embodiment, the further heterologous glucoamylase can have the amino acid sequence of SEQ ID NO: 28, be a variant of the amino acid sequence of SEQ ID NO: 28 having glucoamylase activity or be a fragment of the amino acid sequence of SEQ ID NO: 28 having glucoamylase activity (which can, in an embodiment, correspond to a fragment of the amino acid sequence of SEQ ID NO: 28 lacking its signal sequence, e.g., for example between residues 21 to 632 of SEQ ID NO: 28). For example, the further heterologous glucoamylase can be from a Trametes sp., such as, for example, from Trametes cingulata. In another embodiment, the further heterologous glucoamylase can have the amino acid sequence of SEQ ID NO: 29, be a variant of the amino acid sequence of SEQ ID NO: 29 having glucoamylase activity or be a fragment of the amino acid sequence of SEQ ID NO: 29 having glucoamylase activity (which can, in an embodiment, correspond to a fragment of the amino acid sequence of SEQ ID NO: 29 lacking its signal sequence, e.g., for example between residues 23 to 574 of SEQ ID NO: 29). For example, the further heterologous glucoamylase can be from a Athelia sp., such as, for example, from Athelia rolfsil. In an embodiment, the further heterologous glucoamylase corresponds to Uniprot Q12596 or GenBank Accession Number_BAA08436. In another embodiment, the further heterologous glucoamylase can have the amino acid sequence of SEQ ID NO: 30, be a variant of the amino acid sequence of SEQ ID NO: 30 having glucoamylase activity or be a fragment of the amino acid sequence of SEQ ID NO: 30 having glucoamylase activity (which can, in an embodiment, correspond to a fragment of the amino acid sequence of SEQ ID NO: 30 lacking its signal sequence, e.g., for example between residues 19 to 579 of SEQ ID NO: 30). For example, the further heterologous glucoamylase can be from a Rhizopus sp., such as, for example, from Rhizopus oryzae. In an embodiment, the further heterologous glucoamylase corresponds to Uniprot P07683 or GenBank Accession Number P07683. In another embodiment, the further heterologous glucoamylase can have the amino acid sequence of SEQ ID NO: 31, be a variant of the amino acid sequence of SEQ ID NO: 31 having glucoamylase activity or be a fragment of the amino acid sequence of SEQ ID NO: 31 having glucoamylase activity (which can, in an embodiment, correspond to a fragment of the amino acid sequence of SEQ ID NO: 31 lacking its signal sequence, e.g., for example, between residues 26 and 604 of SEQ ID NO: 31). For example, the further heterologous glucoamylase can be from a Aspergillus sp., such as, for example, from Aspergillus oryzae. In an embodiment, the further heterologous glucoamylase corresponds to Uniprot P36914 or GenBank Accession Number BAA00841. In another embodiment, the further heterologous glucoamylase can have the amino acid sequence of SEQ ID NO: 32, be a variant of the amino acid sequence of SEQ ID NO: 32 having glucoamylase activity or be a fragment of the amino acid sequence of SEQ ID NO: 32 having glucoamylase activity (which can, in an embodiment, correspond to a fragment of the amino acid sequence of SEQ ID NO: 32 lacking its signal sequence, e.g., for example between residues 20 to 612 of SEQ ID NO: 32). In yet another example, the further heterologous glucoamylase can be from Aspergillus awamori. In an embodiment, the further heterologous glucoamylase corresponds to Uniprot Q76L97 or GenBank Accession Number BAD06004. In another embodiment, the further heterologous glucoamylase can have the amino acid sequence of SEQ ID NO: 35, be a variant of the amino acid sequence of SEQ ID NO: 35 having glucoamylase activity or be a fragment of the amino acid sequence of SEQ ID NO: 35 having glucoamylase activity (which can, in an embodiment, correspond to a fragment of the amino acid sequence of SEQ ID NO: 35 lacking its signal sequence, e.g., for example between residues 19 to 639 of SEQ ID NO: 35). In yet another example, the further heterologous glucoamylase can be from Aspergillus niger. In an embodiment, the further heterologous glucoamylase corresponds to Uniprot Q870G8 or GenBank Accession Number AAP04499. In another embodiment, the further heterologous glucoamylase can have the amino acid sequence of SEQ ID NO: 36, be a variant of the amino acid sequence of SEQ ID NO: 36 having glucoamylase activity or be a fragment of the amino acid sequence of SEQ ID NO: 36 having glucoamylase activity (which can, in an embodiment, correspond to a fragment of the amino acid sequence of SEQ ID NO: 36 lacking its signal sequence, e.g., for example between residues 19 to 639 of SEQ ID NO: 36). For example, the further heterologous glucoamylase can be from a Ophiostoma sp., such as, for example, from Ophiostoma floccosum. In an embodiment, the further heterologous glucoamylase corresponds to Uniprot Q06SN2 or GenBank Accession Number ABF72529. In another embodiment, the further heterologous glucoamylase can have the amino acid sequence of SEQ ID NO: 33, be a variant of the amino acid sequence of SEQ ID NO: 33 having glucoamylase activity or be a fragment of the amino acid sequence of SEQ ID NO: 33 having glucoamylase activity (which can, in an embodiment, correspond to a fragment of the amino acid sequence of SEQ ID NO: 33 lacking its signal sequence, e.g., for example between residues 19 to 630 of SEQ ID NO: 33). For example, the further heterologous glucoamylase can be from a Trichocladium sp., such as, for example, from Trichocladium griseum. In an embodiment, the further heterologous glucoamylase corresponds to Uniprot Q12623 or GenBank Accession Number AAA33386. In another embodiment, the further heterologous glucoamylase can have the amino acid sequence of SEQ ID NO: 34, be a variant of the amino acid sequence of SEQ ID NO: 34 having glucoamylase activity or be a fragment of the amino acid sequence of SEQ ID NO: 34 having glucoamylase activity (which can, in an embodiment, correspond to a fragment of the amino acid sequence of SEQ ID NO: 34 lacking its signal sequence, e.g., for example between residues 20 and 620 of SEQ ID NO: 34).

A variant glucoamylase comprises at least one amino acid difference (substitution or addition) when compared to the amino acid sequence of the glucoamylase polypeptide of SEQ ID NO: 1, 3, 11, 13 or 27 to 36 and still exhibits glucoamylase activity. In an embodiment, the variant glucoamylase exhibits at least 10%, 15%, 20%, 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% of the activity of the native or wild-type glucoamylase having the amino acid sequence of SEQ ID NO: 1, 3, 11, 13 or 27 to 36. The glucoamylase variants also have at least 70%, 80%, 81%, 82%, 83%, 84%, 85%, 86%, 87%, 88%, 89%, 90%, 91%, 92%, 93%, 94%, 95%, 96%, 97%, 98% or 99% identity when compared to the wild-type or native glucoamylase having the amino acid sequence of SEQ ID NO: 1, 3, 11, 13 or 27 to 36 over its entire length. The term “percent identity”, as known in the art, is a relationship between two or more polypeptide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variant glucoamylases described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Other conservative amino acid substitutions are known in the art and are included herein. Non-conservative substitutions, such as replacing a basic amino acid with a hydrophobic one, are also well-known in the art.

A variant glucoamylase can also be a conservative variant or an allelic variant. As used herein, a conservative variant refers to alterations in the amino acid sequence that do not adversely affect the biological functions of the starch digesting glucoamylase. A substitution, insertion or deletion is said to adversely affect the polypeptide when the altered sequence prevents or disrupts a biological function associated with the starch digesting glucoamylase (e.g., the hydrolysis of starch into glucose). For example, the overall charge, structure or hydrophobic-hydrophilic properties of the polypeptide can be altered without adversely affecting a biological activity. Accordingly, the amino acid sequence can be altered, for example to render the peptide more hydrophobic or hydrophilic, without adversely affecting the biological activities of the starch digesting glucoamylase.

The present disclosure also provide fragments of the glucoamylase and glucoamylase variants described herein. A fragment comprises at least one less amino acid residue when compared to the amino acid sequence of the catalytic domain or the glucoamylase polypeptide or variant and still possess the enzymatic activity of the full-length glucoamylase. In an embodiment, the glucoamylase fragment exhibits at least 10%, 15%, 20%, 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% activity when compared to the full-length glucoamylase having the amino acid of SEQ ID NO: 1, 3, 11, 13 or 27 to 36 or variants thereof. The glucoamylase fragments can also have at least 70%, 80%, 81%, 82%, 83%, 84%, 85%, 86%, 87%, 88%, 89%, 90%, 91%, 92%, 93%, 94%, 95%, 96%, 97%, 98% or 99% identity when compared to the glucoamylase having the amino acid sequence of SEQ ID NO: 1, 3, 11, 13 or 27 to 36 or variants thereof. The fragment can be, for example, a truncation of one or more amino acid residues at the amino-terminus, the carboxy terminus or both termini of the starch digesting glucoamylase polypeptide or variant. In a specific embodiment, the fragment corresponds to a polypeptide of any one of SEQ ID NO: 27 to 36 to which the signal sequence has been removed. Alternatively or in combination, the fragment can be generated from removing one or more internal amino acid residues. In an embodiment, the glucoamylase fragment has at least 100, 150, 200, 250, 300, 350, 400, 450, 500, 550, 600 or more consecutive amino acids of the glucoamylase having the amino acid sequence of SEQ ID NO: 1, 3, 11, 13 or 27 to 36 or variants thereof.

The heterologous polypeptides having starch or dextrin hydrolase activity described herein are secreted polypeptides. In some embodiments, secreted heterologous polypeptides having starch or dextrin hydrolase activity are released in the culture/fermentation medium and do not remain physically attached to the recombinant yeast host cell. In alternative embodiments, the heterologous starch or dextrin hydrolase of the present disclosure can be secreted, but they remain physically associated with the recombinant yeast host cell. In an embodiment, at least one portion (usually at least one terminus) of the heterologous starch or dextrin hydrolase is bound, covalently, non-covalently and/or electrostatically for example, to the cell wall (and in some embodiments to the cytoplasmic membrane) of the recombinant yeast host cell. For example, the heterologous starch or dextrin hydrolase can be modified to bear one or more transmembrane domains, to have one or more lipid modifications (myristoylation, palmitoylation, farnesylation and/or prenylation), to interact with one or more membrane-associated polypeptide and/or to interactions with the cellular lipid rafts. While the heterologous starch or dextrin hydrolases may not be directly bound to the cell membrane or cell wall (e.g., such as when binding occurs via a tethering moiety), the polypeptide is nonetheless considered a “cell-associated” heterologous polypeptide according to the present disclosure.

In some embodiments, the heterologous starch or dextrin hydrolase can be expressed to be located at and associated to the cell wall of the recombinant yeast host cell. In some embodiments, the heterologous starch or dextrin hydrolase is expressed to be located at and associated to the external surface of the cell wall of the host cell. Recombinant yeast host cells all have a cell wall (which includes a cytoplasmic membrane) defining the intracellular (e.g., internally-facing the nucleus) and extracellular (e.g., externally-facing) environments. The heterologous starch or dextrin hydrolase can be located at (and in some embodiments, physically associated to) the external face of the recombinant yeast host's cell wall and, in further embodiments, to the external face of the recombinant yeast host's cytoplasmic membrane. In the context of the present disclosure, the expression “associated to the external face of the cell wall/cytoplasmic membrane of the recombinant yeast host cell” refers to the ability of the heterologous starch or dextrin hydrolase to physically integrate (in a covalent or non-covalent fashion), at least in part, in the cell wall (and in some embodiments in the cytoplasmic membrane) of the recombinant yeast host cell. The physical integration can be attributed to the presence of, for example, a transmembrane domain on the heterologous polypeptide, a domain capable of interacting with a cytoplasmic membrane polypeptide on the heterologous polypeptide, a post-translational modification made to the heterologous polypeptide (e.g., lipidation), etc.

In some circumstances, it may be warranted to increase or provide cell association to some heterologous starch or dextrin hydrolases because they exhibit insufficient intrinsic cell association or simply lack intrinsic cell association. In such embodiment, it is possible to provide the heterologous starch or dextrin hydrolase as a chimeric construct by combining it with a tethering amino acid moiety which will provide or increase attachment to the cell wall of the recombinant yeast host cell. In such embodiment, the chimeric heterologous polypeptide will be considered “tethered”. It is preferred that the amino acid tethering moiety of the chimeric polypeptide be neutral with respect to the biological activity of the heterologous starch or dextrin hydrolase, e.g., does not interfere with the biological activity (such as, for example, the enzymatic activity) of the heterologous starch or dextrin hydrolase. In some embodiments, the association of the amino acid tethering moiety with the heterologous starch or dextrin hydrolase can increase the biological activity of the heterologous polypeptide (when compared to the non-tethered, “free” form).

In an embodiment, a tethering moiety can be used to be expressed with the heterologous starch or dextrin hydrolase to locate the heterologous polypeptide to the wall of the recombinant yeast host cell. Various tethering amino acid moieties are known art and can be used in the chimeric polypeptides of the present disclosure. The tethering moiety can be a transmembrane domain found on another polypeptide and allow the chimeric polypeptide to have a transmembrane domain. In such embodiment, the tethering moiety can be derived from the FLO1 polypeptide. In still another example, the amino acid tethering moiety can be modified post-translation to include a glycosylphosphatidylinositol (GPI) anchor and allow the chimeric polypeptide to have a GPI anchor. GPI anchors are glycolipids attached to the terminus of a polypeptide (and in some embodiments, to the carboxyl terminus of a polypeptide) which allows the anchoring of the polypeptide to the cytoplasmic membrane of the cell membrane. Tethering amino acid moieties capable of providing a GPI anchor include, but are not limited to those associated with/derived from a SED1 polypeptide, a TIR1 polypeptide, a CWP2 polypeptide, a CCW12 polypeptide, a SPI1 polypeptide, a PST1 polypeptide or a combination of a AGA1 and a AGA2 polypeptide. In an embodiment, the tethering moiety provides a GPI anchor and, in still a further embodiment, the tethering moiety is derived from the SPI1 polypeptide or the CCW12 polypeptide.

The tethering amino acid moiety can be a variant of a known/native tethering amino acid moiety. The tethering amino acid moiety can be a fragment of a known/native tethering amino acid moiety or fragment of a variant of a known/native tethering amino acid moiety.

In embodiments in which an amino acid tethering moiety and/or signal sequence may be desirable, the heterologous polypeptide can be provided as a chimeric polypeptide expressed by the recombinant yeast host cell and having one of the following formulae:

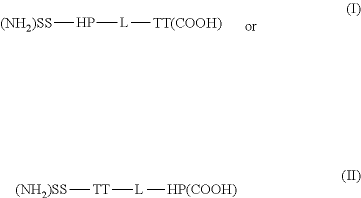

In both of these formulae, the residue “HP” refers to a heterologous polypeptide moiety, the residue “SS” refers to the signal sequence (which cannot have the amino acid sequence of SEQ ID NO: 5), the residue “L” refers to the presence of an optional linker, and the residue “TT” refers to an optional amino acid tethering moiety. In the chimeric polypeptides of formula (I), the amino (NH₂ or N) terminus of the amino acid tether is located (directly or indirectly) at the carboxyl (COOH or C) terminus of the heterologous starch or dextrin hydrolase moiety. In the chimeric polypeptides of formula (I), the amino (NH₂ or N) terminus of the heterologous starch or dextrin hydrolase moiety is located (directly or indirectly) at the carboxyl (COOH or C) terminus of the signal sequence. In the chimeric polypeptides of formula (II), the carboxy (COOH or C) terminus of the amino acid tether is located (directly or indirectly) at the amino (NH₂ or N) terminus of the heterologous starch or dextrin hydrolase moiety. In the chimeric polypeptides of formula (II), the carboxy (COOH or C) terminus of signal sequence is located (directly or indirectly) at the amino (NH₂ or N) terminus of the amino acid tether. Embodiments of chimeric tethered heterologous polypeptides have been disclosed in WO2018/167670 and are included herein in their entirety.

The heterologous nucleic acid molecule can include a third polynucleotide including a promoter capable of controlling the expression of the first and second polynucleotide. In such embodiment, the promoter and the polynucleotide coding for the signal sequence and the heterologous polypeptide are operatively linked to one another. In the context of the present disclosure, the expressions “operatively linked” or “operatively associated” refers to fact that the promoter is physically associated to the first and second polynucleotide in a manner that allows, under certain conditions, for expression of the heterologous polypeptide from the heterologous nucleic acid molecule. In an embodiment, the promoter can be located upstream (5′) of the nucleic acid sequence coding for the heterologous polypeptide. In still another embodiment, the promoter can be located downstream (3′) of the nucleic acid sequence coding for the heterologous polypeptide. In the context of the present disclosure, one or more than one promoter can be included in the nucleic acid molecule. When more than one promoter is included in the nucleic acid molecule, each of the promoters is operatively linked to the nucleic acid sequence coding for the polypeptide. The promoters can be located, in view of the nucleic acid molecule coding for the polypeptide, upstream, downstream as well as both upstream and downstream.

“Promoter” refers to a DNA fragment capable of controlling the expression of a coding sequence or functional RNA. The term “expression,” as used herein, refers to the transcription and stable accumulation of sense (mRNA) from the heterologous nucleic acid molecule described herein. Expression may also refer to translation of mRNA into a polypeptide. Promoters may be derived in their entirety from a native gene, or be composed of different elements derived from different promoters found in nature, or even comprise synthetic DNA segments. It is understood by those skilled in the art that different promoters may direct the expression at different stages of development, or in response to different environmental or physiological conditions. Promoters which cause a gene to be expressed in most cells at most times at a substantial similar level are commonly referred to as “constitutive promoters”. It is further recognized that since in most cases the exact boundaries of regulatory sequences have not been completely defined, DNA fragments of different lengths may have identical promoter activity. A promoter is generally bounded at its 3′ terminus by the transcription initiation site and extends upstream (5′ direction) to include the minimum number of bases or elements necessary to initiate transcription at levels detectable above background. Within the promoter will be found a transcription initiation site (conveniently defined for example, by mapping with nuclease S1), as well as polypeptide binding domains (consensus sequences) responsible for the binding of the polymerase.

The promoter can be heterologous to the nucleic acid molecule encoding the heterologous polypeptide. The promoter can be heterologous or derived from a strain being from the same genus or species as the recombinant host cell. In an embodiment, the promoter is derived from the same genus or species of the yeast host cell and the polypeptide is derived from different genera that the host cell. One or more promoters can be used to allow the expression of the polypeptides in the recombinant yeast host cell.

In some embodiments, the recombinant yeast host cell is a facultative anaerobe, such as Saccharomyces cerevisiae. For facultative anaerobes, cells tend to propagate or ferment depending on the availability of oxygen. In a fermentation process, yeast cells are generally allowed to propagate before fermentation is conducted. In some embodiments, the promoter preferentially initiates transcription during a propagation phase such that the heterologous polypeptides (variants or fragments) are expressed during the propagation phase. As used in the context of the present disclosure, the expression “propagation phase” refers to an expansion phase of a commercial process in which the yeasts are propagated under aerobic conditions. In some instances, the propagated biomass can be used in a following fermenting step (e.g. under anaerobic conditions) to maximize the production of one or more desired metabolites or fermentation products.

The heterologous nucleic acid molecule can include a promoter (or a combination of promoters) capable of allowing the expression of the heterologous polypeptide during propagation (and optionally during fermentation). This embodiment will allow the accumulation of the polypeptide associated with the recombinant yeast host cell prior to any subsequent use, for example in liquefaction or fermentation. In some embodiments, the promoter substantially limits the expression of the polypeptide during the propagation phase.

The expression of the polypeptides during the propagation phase may provide sufficient expression such that the polypeptide or the recombinant yeast cells may be added during the liquefaction of starch, thereby providing yeast cells with sufficient nutrients to undergo metabolic processing. The promoters can be native or heterologous to the heterologous gene encoding the heterologous polypeptide. The promoters that can be included in the heterologous nucleic acid molecule can be constitutive or inducible promoters. Constitutive promoters include, but are not limited to, tef2p (e.g., the promoter of the tef2 gene, a variant thereof or a fragment thereof), cwp2p (e.g., the promoter of the cwp2 gene, a variant thereof or a fragment thereof), ssa1p (e.g., the promoter of the ssa1 gene, a variant thereof), eno1p (e.g., the promoter of the eno1 gene, variant thereof or a fragment thereof), hxk1 (e.g., the promoter of the hxk1 gene, a variant thereof or a fragment thereof) and/or pgk1p (e.g., the promoter of the pgk1 gene, a variant thereof or a fragment thereof). Inducible promoters include, but are not limited to glucose-regulated promoters (e.g., the promoter of the hxt7 gene (referred to as hxt7p), a functional variant or a functional fragment thereof; the promoter of the ctt1 gene (referred to as ctt1p), a functional variant or a functional fragment thereof; the promoter of the glo1 gene (referred to as glo1p), a functional variant or a functional fragment thereof; the promoter of the ygp1 gene (referred to as ygp1p), a functional variant or a functional fragment thereof; the promoter of the gsy2 gene (referred to as gsy2p), a functional variant or a functional fragment thereof), molasses-regulated promoters (e.g., the promoter of the mol1 gene (referred to as mol1p), a functional variant or a functional fragment thereof), heat shock-regulated promoters (e.g., the promoter of the glo1 gene (referred to as glo1p), a functional variant or a functional fragment thereof; the promoter of the sti1 gene (referred to as sti1p), a functional variant or a functional fragment thereof; the promoter of the ygp1 gene (referred to as ygp1p), a functional variant or a functional fragment thereof; the promoter of the gsy2 gene (referred to as gsy2p), a functional variant or a functional fragment thereof), oxidative stress response promoters (e.g., the promoter of the cup1 gene (referred to as cup1p), a functional variant or a functional fragment thereof; the promoter of the ctt1 gene (referred to as ctt1p), a functional variant or a functional fragment thereof; the promoter of the trx2 gene (referred to as trx2p), a functional variant or a functional fragment thereof; the promoter of the gpd1 gene (referred to as gpd1p), a functional variant or a functional fragment thereof; the promoter of the hsp12 gene (referred to as hsp12p), a functional variant or a functional fragment thereof), osmotic stress response promoters (e.g., the promoter of the ctt1 gene (referred to as ctt1p), a functional variant or a functional fragment thereof; the promoter of the glo1 gene (referred to as glo1p), a functional variant or a functional fragment thereof; the promoter of the gpd1 gene (referred to as gpd1p), a functional variant or a functional fragment thereof; the promoter of the ygp1 gene (referred to as ygp1p), a functional variant or a functional fragment thereof), nitrogen-regulated promoters (e.g., the promoter of the ygp1 gene (referred to as ygp1p), a functional variant or a functional fragment thereof) and the promoter of the adh1 gene (referred to as adh1p), a functional variant or a functional fragment thereof) and/or a molasses-regulated promoter (e.g., the promoter of the tir1 gene (referred to as tir1p), a functional variant or a functional fragment thereof).

Promoters that can be included in the heterologous nucleic acid molecule of the present disclosure include, without limitation, the promoter of the tdh1 gene (referred to as tdh1p, a functional variant or a functional fragment thereof), of the hor7 gene (referred to as hor7p, a functional variant or a functional fragment thereof), of the hsp150 gene (referred to as hsp150p, a functional variant or a functional fragment thereof), of the hxt7 gene (referred to as hxt7p, a functional variant or a functional fragment thereof), of the gpm1 gene (referred to as gpm1p, a functional variant or a functional fragment thereof), of the pgk1 gene (referred to as pgk1p, a functional variant or a functional fragment thereof), of the stl1 gene (referred to as stl1p, a functional variant or a functional fragment thereof) and/or of the tef2 gen (referred to as tef2p, a functional variant or a functional fragment thereof). In an embodiment, the promoter is or comprises the tef2p. In still another embodiment, the promoter comprises or consists essentially of the tdh1p and the hor7p. In a further embodiment, the promoter is the thd1p. In another embodiment, the promoter is the adh1p.

In the context of the present disclosure, the expression “functional fragment of a promoter” when used in combination to a promoter refers to a shorter nucleic acid sequence than the native promoter which retain the ability to control the expression of the nucleic acid sequence encoding the polypeptides during the propagation phase of the recombinant yeast host cells. Usually, functional fragments are either 5′ and/or 3′ truncation of one or more nucleic acid residue from the native promoter nucleic acid sequence.

In some embodiments, the heterologous nucleic acid molecules include one or a combination of terminator sequence(s) to end the translation of the heterologous polypeptide (or of the chimeric polypeptide comprising same). The terminator can be native or heterologous to the nucleic acid sequence encoding the heterologous polypeptide or its corresponding chimera. In some embodiments, one or more terminators can be used. In some embodiments, the terminator comprises the terminator derived from is from the dit1 gene (dit1t, a functional variant or a functional fragment thereof), from the idp1 gene (idp1t, a functional variant or a functional fragment thereof), from the gpm1 gene (gpm1t, a functional variant or a functional fragment thereof), from the pma1 gene (pam1t, a functional variant or a functional fragment thereof), from the tdh3 gene (tdh3t, a functional variant or a functional fragment thereof), from the hxt2 gene (a functional variant or a functional fragment thereof), from the adh3 gene (adh3t, a functional variant or a functional fragment thereof), and/or from the ira2 gene (ira2t, a functional variant or a functional fragment thereof). In an embodiment, the terminator comprises or is derived from the dit1 gene (dit1t, a functional variant or a functional fragment thereof). In another embodiment, the terminator comprises or is derived adh3t and/or idp1t. In the context of the present disclosure, the expression “functional variant of a terminator” refers to a nucleic acid sequence that has been substituted in at least one nucleic acid position when compared to the native terminator which retain the ability to end the expression of the nucleic acid sequence coding for the heterologous polypeptide or its corresponding chimera. In the context of the present disclosure, the expression “functional fragment of a terminator” refers to a shorter nucleic acid sequence than the native terminator which retain the ability to end the expression of the nucleic acid sequence coding for the heterologous polypeptide or its corresponding chimera.

In some embodiments, the recombinant host cell comprises a genetic modification (e.g., one or more heterologous nucleic acid molecule) allowing the recombinant expression of the polypeptide having starch or dextrin hydrolase activity. In such embodiment, a heterologous nucleic acid molecule encoding the polypeptide having starch or dextrin hydrolase activity can be introduced in the recombinant host to express the polypeptide having starch or dextrin hydrolase activity. The expression of the polypeptide having starch or dextrin hydrolase activity can be constitutive or induced.

In some embodiments, the recombinant host cell comprises a further genetic modification (e.g., the introduction of one or more heterologous nucleic acid molecule) allowing the recombinant expression of the polypeptide having starch digesting alpha-amylase activity. In such embodiment, a heterologous nucleic acid molecule encoding the polypeptide having starch digesting alpha-amylase activity can be introduced in the recombinant host to express the polypeptide having starch digesting alpha-amylase activity activity. The expression of the polypeptide having starch digesting alpha-amylase activity can be constitutive or induced. For example, the heterologous alpha-amylase can be from a Rhizomucor sp., such as, for example, from Rhizomucor pusillus. In an embodiment, the heterologous alpha-amylase corresponds to Uniprot M9TI89. In another embodiment, the heterologous alpha-amylase can have the amino acid sequence of SEQ ID NO: 17, be a variant of the amino acid sequence of SEQ ID NO: 17 having alpha-amylase activity or be a fragment of the amino acid sequence of SEQ ID NO: 17 having alpha-amylase activity (which can, in an embodiment, correspond to a fragment of the amino acid sequence of SEQ ID NO: 17 lacking its signal sequence, e.g., for example, between residues 22 and 471 of SEQ ID NO: 17). For example, the heterologous alpha-amylase can be from a Aspergillus sp., such as, for example, from Aspergillus luchuensis. In an embodiment, the heterologous alpha-amylase corresponds to Uniprot A0A146F6W4 or to GenBank Accession Number GAT21778. In another embodiment, the heterologous alpha-amylase can have the amino acid sequence of SEQ ID NO: 18, be a variant of the amino acid sequence of SEQ ID NO: 18 having alpha-amylase activity or be a fragment of the amino acid sequence of SEQ ID NO: 18 having alpha-amylase activity (which can, in an embodiment, correspond to a fragment of the amino acid sequence of SEQ ID NO: 18 lacking its signal sequence, e.g., for example, between residues 22 to 615 of SEQ ID NO: 18). In an embodiment, the heterologous alpha-amylase corresponds to Uniprot 013296 or to GenBank Accession Number BAA22993. In another embodiment, the heterologous alpha-amylase can have the amino acid sequence of SEQ ID NO: 26, be a variant of the amino acid sequence of SEQ ID NO: 26 having alpha-amylase activity or be a fragment of the amino acid sequence of SEQ ID NO: 26 having alpha-amylase activity (which can, in an embodiment, correspond to a fragment of the amino acid sequence of SEQ ID NO: 26 lacking its signal sequence, e.g., for example between residues 22 to 640 of SEQ ID NO: 26). For example, the heterologous alpha-amylase can be from Aspergillus oryzae. In an embodiment, the heterologous alpha-amylase corresponds to Uniprot Q2UIS5 or to GenBank Accession Number XP_001820542. In another embodiment, the heterologous alpha-amylase can have the amino acid sequence of SEQ ID NO: 19, be a variant of the amino acid sequence of SEQ ID NO: 19 having alpha-amylase activity or be a fragment of the amino acid sequence of SEQ ID NO: 19 having alpha-amylase activity (which can, in an embodiment, correspond to a fragment of the amino acid sequence of SEQ ID NO: 19 lacking its signal sequence, e.g., for example between residues 24 to 549 of SEQ ID NO: 19). For example, the heterologous alpha-amylase can be from Aspergillus niger. In an embodiment, the heterologous alpha-amylase corresponds to Uniprot A2QTS4 or to GenBank Accession Number XP_001393626. In another embodiment, the heterologous alpha-amylase can have the amino acid sequence of SEQ ID NO: 21, be a variant of the amino acid sequence of SEQ ID NO: 21 having alpha-amylase activity or be a fragment of the amino acid sequence of SEQ ID NO: 21 having alpha-amylase activity (which can, in an embodiment, correspond to a fragment of the amino acid sequence of SEQ ID NO: 21 lacking its signal sequence, e.g., for example between residues 26 to 555 of SEQ ID NO: 21). In an embodiment, the heterologous alpha-amylase corresponds to Uniprot A2R6F9 or to GenBank Accession Number XP_001397301. In another embodiment, the heterologous alpha-amylase can have the amino acid sequence of SEQ ID NO: 22, be a variant of the amino acid sequence of SEQ ID NO: 22 having alpha-amylase activity or be a fragment of the amino acid sequence of SEQ ID NO: 22 having alpha-amylase activity (which can, in an embodiment, correspond to a fragment of the amino acid sequence of SEQ ID NO: 22 lacking its signal sequence, e.g., for example between residues 23 and 567 of SEQ ID NO: 22). In an embodiment, the heterologous alpha-amylase corresponds to GenBank Accession Number XP_001395328. In another embodiment, the heterologous alpha-amylase can have the amino acid sequence of SEQ ID NO: 23, be a variant of the amino acid sequence of SEQ ID NO: 23 having alpha-amylase activity or be a fragment of the amino acid sequence of SEQ ID NO: 23 having alpha-amylase activity (which can, in an embodiment, correspond to a fragment of the amino acid sequence of SEQ ID NO: 23 lacking its signal sequence, e.g., for example between residues 30 and 550 of SEQ ID NO: 23). In an embodiment, the heterologous alpha-amylase corresponds to Uniprot A0A370BQ30 or to GenBank Accession Number RDH15462. In another embodiment, the heterologous alpha-amylase can have the amino acid sequence of SEQ ID NO: 24, be a variant of the amino acid sequence of SEQ ID NO: 24 having alpha-amylase activity or be a fragment of the amino acid sequence of SEQ ID NO: 24 having alpha-amylase activity (which can, in an embodiment, correspond to a fragment of the amino acid sequence of SEQ ID NO: 24 lacking its signal sequence, e.g., for example between residues 17 and 524 of SEQ ID NO: 24). For example, the heterologous alpha-amylase can be from Aspergillus fischeri. In an embodiment, the heterologous alpha-amylase corresponds to Uniprot A1CYB1 or to GenBank Accession Number XP_001265628. In another embodiment, the heterologous alpha-amylase can have the amino acid sequence of SEQ ID NO: 25, be a variant of the amino acid sequence of SEQ ID NO: 25 having alpha-amylase activity or be a fragment of the amino acid sequence of SEQ ID NO: 25 having alpha-amylase activity (which can, in an embodiment, correspond to a fragment of the amino acid sequence of SEQ ID NO: 25 lacking its signal sequence, e.g., for example between residues 24 to 632 of SEQ ID NO: 25). For example, the heterologous alpha-amylase can be from a Homo sp., such as, for example, from Homo sapiens. In an embodiment, the heterologous alpha-amylase corresponds to GenBank Accession Number 1B2Y_A. In another embodiment, the heterologous alpha-amylase can have the amino acid sequence of SEQ ID NO: 20, be a variant of the amino acid sequence of SEQ ID NO: 20 having alpha-amylase activity or be a fragment of the amino acid sequence of SEQ ID NO: 20 having alpha-amylase activity (which can, in an embodiment, correspond to a fragment of the amino acid sequence of SEQ ID NO: 20 lacking its signal sequence, e.g., for example between residues 20 to 515 of SEQ ID NO: 20).

A variant alpha-amylase comprises at least one amino acid difference (substitution or addition) when compared to the amino acid sequence of the alpha-amylase polypeptide of SEQ ID NO: 17 to 26 and still exhibits alpha-amylase activity. In an embodiment, the variant alpha-amylase exhibits at least 10%, 15%, 20%, 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% of the activity of the native or wild-type alpha-amylase having the amino acid sequence of SEQ ID NO: 17 to 26. The alpha-amylase variants also have at least 70%, 80%, 81%, 82%, 83%, 84%, 85%, 86%, 87%, 88%, 89%, 90%, 91%, 92%, 93%, 94%, 95%, 96%, 97%, 98% or 99% identity when compared to the wild-type or native alpha-amylase having the amino acid sequence of SEQ ID NO: 17 to 26 over its entire length. The term “percent identity”, as known in the art, is a relationship between two or more polypeptide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variant alpha-amylases described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Other conservative amino acid substitutions are known in the art and are included herein. Non-conservative substitutions, such as replacing a basic amino acid with a hydrophobic one, are also well-known in the art.

A variant alpha-amylase can also be a conservative variant or an allelic variant. As used herein, a conservative variant refers to alterations in the amino acid sequence that do not adversely affect the biological functions of the starch digesting alpha-amylase. A substitution, insertion or deletion is said to adversely affect the polypeptide when the altered sequence prevents or disrupts a biological function associated with the starch digesting alpha-amylase (e.g., the hydrolysis of starch into glucose). For example, the overall charge, structure or hydrophobic-hydrophilic properties of the polypeptide can be altered without adversely affecting a biological activity. Accordingly, the amino acid sequence can be altered, for example to render the peptide more hydrophobic or hydrophilic, without adversely affecting the biological activities of the starch digesting alpha-amylase.

The present disclosure also provide fragments of the alpha-amylases and alpha-amylases variants described herein. A fragment comprises at least one less amino acid residue when compared to the amino acid sequence of the catalytic domain or the alpha-amylase polypeptide or variant and still possess the enzymatic activity of the full-length alpha-amylase. In an embodiment, the alpha-amylase fragment exhibits at least 10%, 15%, 20%, 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% activity when compared to the full-length alpha-amylase having the amino acid of SEQ ID NO: 17 to 26 or variants thereof. The alpha-amylase fragments can also have at least 70%, 80%, 81%, 82%, 83%, 84%, 85%, 86%, 87%, 88%, 89%, 90%, 91%, 92%, 93%, 94%, 95%, 96%, 97%, 98% or 99% identity when compared to the alpha-amylase having the amino acid sequence of SEQ ID NO: 17 to 26 or variants thereof. The fragment can be, for example, a truncation of one or more amino acid residues at the amino-terminus, the carboxy terminus or both termini of the starch digesting alpha-amylase or variant. In a specific embodiment, the fragment corresponds to a polypeptide of any one of SEQ ID NO: 17 to 26 to which the signal sequence has been removed. Alternatively or in combination, the fragment can be generated from removing one or more internal amino acid residues. In an embodiment, the alpha-amylase fragment has at least 100, 150, 200, 250, 300, 350, 400, 450, 500, 550, 600 or more consecutive amino acids of the alpha-amylase having the amino acid sequence of SEQ ID NO: 17 to 26 or variants thereof.

The alpha-amylase can be expressed using its native signal sequence or can be expressed using a heterologous signal sequence. In embodiments the heterologous signal sequence associated with the alpha-amylase can have the amino acid sequence of SEQ ID NO: 5 or the section spanning residues 1 to 21 of SEQ ID NO: 17, 1 to 21 of SEQ ID NO: 18, 1 to 23 of SEQ ID NO: 19, 1 to 19 of SEQ ID NO: 20, 1 to 25 of SEQ ID NO: 21, 1 to 22 of SEQ ID NO: 22, 1 to 29 of SEQ ID NO: 23, 1 to 16 of SEQ ID NO: 24, 1 to 23 of SEQ ID NO: 25, 1 to 21 of SEQ ID NO: 26, 1 to 17 of SEQ ID NO: 27, 1 to 20 of SEQ ID NO: 28, 1 to 22 of SEQ ID NO: 29, 1 to 18 of SEQ ID NO: 30, 1 to 25 of SEQ ID NO: 31, 1 to 19 of SEQ ID NO: 32, 1 to 18 of SEQ ID NO: 33, 1 to 19 of SEQ ID NO: 34, 1 to 18 of SEQ ID NO: 35, 1 to 18 of SEQ ID NO: 36 as well as variants and fragments thereof.

### Process for Saccharification and Fermentation of a Biomass

The recombinant yeast host cells described herein can be used in saccharification for improving the hydrolysis of a biomass and, in some embodiments, the production of a fermentation product from the biomass. In some embodiments, the recombinant yeast host cells of the present disclosure maintain their robustness during saccharification and fermentation in the presence of a stressor such as, for example, lactic acid, formic acid and/or a bacterial contamination (that can be associated, in some embodiments, with an increase in lactic acid during fermentation), a decrease in pH, a reduction in aeration, elevated temperatures or a combination of these conditions.

The fermented product intended to be obtained during the fermentation can be an alcohol, such as, for example, ethanol, isopropanol, n-propanol, 1-butanol, methanol, acetone, 1,3-propanediol and/or 1,2-propanediol. In an embodiment, the fermented product is ethanol.

In the process, the biomass that can be hydrolyzed (and optionally fermented) with the recombinant yeast host cells. Such biomass includes any type of biomass known in the art and described herein. For example, the biomass can include, but is not limited to, starch, sugar and lignocellulosic materials. Starch materials can include, but are not limited to, mashes such as corn, wheat, rye, barley, rice, or milo. Sugar materials can include, but are not limited to, sugar beets, artichoke tubers, sweet sorghum, molasses or cane. The terms “lignocellulosic material”, “lignocellulosic substrate” and “cellulosic biomass” mean any type of substrate comprising cellulose, hemicellulose, lignin, or combinations thereof, such as but not limited to woody biomass, forage grasses, herbaceous energy crops, non-woody-plant biomass, agricultural wastes and/or agricultural residues, forestry residues and/or forestry wastes, paper-production sludge and/or waste paper sludge, waste-water-treatment sludge, municipal solid waste, corn fiber from wet and dry mill corn ethanol plants and sugar-processing residues. The terms “hemicellulosics”, “hemicellulosic portions” and “hemicellulosic fractions” mean the non-lignin, non-cellulose elements of lignocellulosic material, such as but not limited to hemicellulose (i.e., comprising xyloglucan, xylan, glucuronoxylan, arabinoxylan, mannan, glucomannan and galactoglucomannan), pectins (e.g., homogalacturonans, rhamnogalacturonan I and II, and xylogalacturonan) and proteoglycans (e.g., arabinogalactan-polypeptide, extensin, and pro line-rich polypeptides).

In a non-limiting example, the lignocellulosic material can include, but is not limited to, woody biomass, such as recycled wood pulp fiber, sawdust, hardwood, softwood, and combinations thereof; grasses, such as switch grass, cord grass, rye grass, reed canary grass, miscanthus, or a combination thereof; sugar-processing residues, such as but not limited to sugar cane bagasse; agricultural wastes, such as but not limited to rice straw, rice hulls, barley straw, corn cobs, cereal straw, wheat straw, canola straw, oat straw, oat hulls, and corn fiber; stover, such as but not limited to soybean stover, corn stover; succulents, such as but not limited to, agave; and forestry wastes, such as but not limited to, recycled wood pulp fiber, sawdust, hardwood (e.g., poplar, oak, maple, birch, willow), softwood, or any combination thereof. Lignocellulosic material may comprise one species of fiber; alternatively, lignocellulosic material may comprise a mixture of fibers that originate from different lignocellulosic materials. Other lignocellulosic materials are agricultural wastes, such as cereal straws, including wheat straw, barley straw, canola straw and oat straw; corn fiber; stovers, such as corn stover and soybean stover; grasses, such as switch grass, reed canary grass, cord grass, and miscanthus; or combinations thereof.

Substrates for cellulose activity assays can be divided into two categories, soluble and insoluble, based on their solubility in water. Soluble substrates include cellodextrins or derivatives, carboxymethyl cellulose (CMC), or hydroxyethyl cellulose (HEC). Insoluble substrates include crystalline cellulose, microcrystalline cellulose (Avicel), amorphous cellulose, such as phosphoric acid swollen cellulose (PASC), dyed or fluorescent cellulose, and pretreated lignocellulosic biomass. These substrates are generally highly ordered cellulosic material and thus only sparingly soluble.

It will be appreciated that suitable lignocellulosic material may be any feedstock that contains soluble and/or insoluble cellulose, where the insoluble cellulose may be in a crystalline or non-crystalline form. In various embodiments, the lignocellulosic biomass comprises, for example, wood, corn, corn stover, sawdust, bark, molasses, sugarcane, leaves, agricultural and forestry residues, grasses such as switchgrass, ruminant digestion products, municipal wastes, paper mill effluent, newspaper, cardboard or combinations thereof.

Paper sludge is also a viable feedstock for lactate or acetate production. Paper sludge is solid residue arising from pulping and paper-making, and is typically removed from process wastewater in a primary clarifier. The cost of disposing of wet sludge is a significant incentive to convert the material for other uses, such as conversion to ethanol. Processes provided by the present invention are widely applicable. Moreover, the hydrolyzed biomass may be used to produce ethanol or higher value added chemicals, such as organic acids, aromatics, esters, acetone and polymer intermediates.

The process of the present disclosure comprise contacting the recombinant host cells comprising the heterologous polypeptide having starch or dextrin hydrolase activity described herein with a biomass so as to allow the hydrolysis of at least a part of the biomass and the conversion of the biomass (at least in part) into a fermentation product (e.g., an alcohol such as ethanol). In some embodiments, the biomass to be hydrolyzed/fermented is a lignocellulosic biomass and, in some embodiments, it comprises starch (in a gelatinized or raw form). In an embodiment, the biomass to be hydrolyzed/fermented is raw starch. In other embodiments, the biomass to be hydrolyzed/fermented is derived from corn, potato, cassava, rice, or buckwheat. In preferred embodiments, the biomass is derived from corn, such as in the form of corn mash. The process can include, in some embodiments, heating the lignocellulosic biomass prior to fermentation to provide starch in a gelatinized form. In another embodiment, the biomass comprises or is derived from sugar cane.

The fermentation process can be performed at temperatures of at least about 20° C., about 21° C., about 22° C., about 23° C., about 24° C., about 25° C., about 26° C., about 27° C., about 28° C., about 29° C., about 30° C., about 31° C., about 32° C., about 33°, about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., or about 50° C. In some embodiments, the production of ethanol from cellulose can be performed, for example, at temperatures above about 30° C., about 31° C., about 32° C., about 33° C., about 34° C., about 35° C., about 36° C., about 37° C., about 38° C., about 39° C., about 40° C., about 41° C., about 42° C., or about 43° C., or about 44° C., or about 45° C., or about 50° C. In some embodiments, the recombinant microbial host cell can produce ethanol from cellulose at temperatures from about 30° C. to 60° C., about 30° C. to 55° C., about 30° C. to 50° C., about 40° C. to 60° C., about 40° C. to 55° C. or about 40° C. to 50° C.

In some embodiments, the liquefaction of starch occurs in the presence of recombinant yeast host cells described herein. In some embodiments, the liquefaction of starch is maintained at a temperature of between about 70° C.-105° C. to allow for proper gelatinization and hydrolysis of the starch. In an embodiment, the liquefaction occurs at a temperature of at least about 70° C., 75° C., 80° C., 85° C., 90° C., 95° C., 100° C. or 105° C. Alternatively or in combination, the liquefaction occurs at a temperate of no more than about 105° C., 100° C., 95° C., 90° C., 85° C., 80° C., 75° C. or 70° C. In yet another embodiment, the liquefaction occurs at a temperature between about 80° C. and 85° C. (which can include a thermal treatment spike at 105° C.).

In some embodiments, the process can be used to produce ethanol at a particular rate. For example, in some embodiments, ethanol is produced at a rate of at least about 0.1 mg per hour per liter, at least about 0.25 mg per hour per liter, at least about 0.5 mg per hour per liter, at least about 0.75 mg per hour per liter, at least about 1.0 mg per hour per liter, at least about 2.0 mg per hour per liter, at least about 5.0 mg per hour per liter, at least about 10 mg per hour per liter, at least about 15 mg per hour per liter, at least about 20.0 mg per hour per liter, at least about 25 mg per hour per liter, at least about 30 mg per hour per liter, at least about 50 mg per hour per liter, at least about 100 mg per hour per liter, at least about 200 mg per hour per liter, at least about 300 mg per hour per liter, at least about 400 mg per hour per liter, at least about 500 mg per hour per liter, at least about 600 mg per hour per liter, at least about 700 mg per hour per liter, at least about 800 mg per hour per liter, at least about 900 mg per hour per liter, at least about 1 g per hour per liter, at least about 1.5 g per hour per liter, at least about 2 g per hour per liter, at least about 2.5 g per hour per liter, at least about 3 g per hour per liter, at least about 3.5 g per hour per liter, at least about 4 g per hour per liter, at least about 4.5 g per hour per liter, at least about 5 g per hour per liter, at least about 5.5 g per hour per liter, at least about 6 g per hour per liter, at least about 6.5 g per hour per liter, at least about 7 g per hour per liter, at least about 7.5 g per hour per liter, at least about 8 g per hour per liter, at least about 8.5 g per hour per liter, at least about 9 g per hour per liter, at least about 9.5 g per hour per liter, at least about 10 g per hour per liter, at least about 10.5 g per hour per liter, at least about 11 g per hour per liter, at least about 11.5 g per hour per liter, at least about 12 g per hour per liter, at least about 12.5 g per hour per liter, at least about 13 g per hour per liter, at least about 13.5 g per hour per liter, at least about 14 g per hour per liter, at least about 14.5 g per hour per liter or at least about 15 g per hour per liter.

Ethanol yield per fermentation batch can be measured using one of at least four methods: 1) measurement of total gallons of ethanol distilled per bushel of corn ground for the processing of each fermentation batch; 2) measurement of ethanol concentration (titer) in the fermentation beer soluble fraction (supernatant) at the end of fermentation before the beer is pumped to the beer well, normalized to the average total solids (total dry matter) concentration of the liquefact used to fill the fermentation batch (“ethanol yield per liquefact solids”); 3) measurement of ethanol concentration in the fermentation beer soluble fraction (supernatant) at the end of fermentation before the beer is pumped to the beer well, normalized to the average total solids (total dry matter) concentration of the slurry used to produce the liquefact filling the fermentation batch (“ethanol yield per slurry solids”); and 4) measurement of ethanol concentration in the fermentation beer soluble fraction (supernatant) at the end of fermentation before the beer is pumped to the beer well, normalized to the average total solids (total dry matter) concentration of the liquefact mash filling the fermentation batch, with the liquefact mash total solids (total dry matter) adjusted for the recycled unfermented total solids (total dry matter) in the recycled thin stillage (backset): “ethanol yield per fermentable mash solids.” It is possible to track total ethanol production and corn consumption on a daily basis, rather than on a per batch basis, and so the second, third, and fourth ethanol yield measurement methods per fermentation batch are most commonly used to assess batch ethanol yield. Where changes in the concentration of unfermented total solids in the recycled thin stillage or changes in the relative volumetric flow rate of the recycled thin stillage to the total liquefact flow rate may occur, the fourth method of measuring ethanol yield per fermentation batch is preferable (ethanol yield per fermentable mash solids). For production facilities that do not frequently measure total solids (total dry matter) of recycled thin stillage, or that do not track the relative volumetric flow rate of recycled thin stillage to that of the total liquefact on a per batch basis, the second or third ethanol yield measurement methods are typically applied.

Samples of fermentation beer can be taken periodically (often every 6 h, 10 h, or 12 h) over the course of each fermentation batch to evaluate the progress of the fermentation batch as glucose is converted to ethanol, and then at the end of the fermentation batch (at fermentation drop) as part of evaluating the final ethanol yield for the batch. Samples can either be centrifuged to remove suspended solids with the supernatant collected, or allowed to drip through filter paper to remove suspended solids with the filtrate collected. The sample supernatant or filtrate can be filtered (through a 0.22 μm pore size syringe filter cartridge for example), and the clarified filtrate collected in an HPLC vial. The clarified filtrate is analyzed using high-performance liquid chromatography (HPLC) to measure the concentration of soluble fermentation components including: ethanol, glycerol, glucose (dextrose), lactic acid, acetic acid, and glucose di- and trisaccharides (DP2 and DP3). Higher molecular weight glucose oligomers (often referred to as “DP4+”) are typically measured as part of a single aggregate peak that elutes with the void volume of the HPLC column. The sum of the concentrations of glucose, DP2, DP3, and DP4+ as measured in the beer soluble fraction by HPLC is referred to as the “total residual sugar” concentration.

Ethanol production can be measured using any method known in the art. For example, the quantity of ethanol in fermentation samples can be assessed using HPLC analysis. Many ethanol assay kits are commercially available that use, for example, alcohol oxidase enzyme based assays.

In some embodiments, the process can be used in the presence of a stressor such as low pH. For example, the stressor is a pH of 7.0 or lower, 6.5 or lower, 6.0 or lower, 5.5 or lower, 5.0 or lower, 4.8 or lower, 4.6 or lower, 4.4 or lower, 4.2 or lower, 4.0 or lower, 3.8 or lower, 3.6 or lower, 3.4 or lower, 3.2 or lower, or 3.0 or lower.

As shown in the Examples, recombinant yeast host cells expressing the heterologous glucoamylase exhibits enhanced robustness compared to yeast host cells expressing other known glucoamylases. In specific embodiments of a recombinant yeast host cell expressing a heterologous glucoamylase, fermentation with the recombinant yeast cell yielded higher ethanol titers than recombinant yeast host cells expressing other heterologous glucoamylases. In some embodiments, the recombinant yeast host cell expressing heterologous glucoamylase yielded greater than 10 mg/L increase, greater than 25 mg/L increase, greater than 50 mg/L increase, greater than 100 mg/L increase, greater than 200 mg/L increase, greater than 300 mg/L increase, greater than 400 mg/L increase, greater than 500 mg/L increase, greater than 600 mg/L increase, greater than 700 mg/L increase, greater than 800 mg/L increase, greater than 900 mg/L increase, or greater than 1 g/L increase) in ethanol production at low pH values and in corn fermentation when compared to recombinant yeast host cells expressing other heterologous glucoamylases.

In the process described herein, it is possible to add an exogenous source (e.g., to dose) of an enzyme to facilitate saccharification or improve fermentation yield. As such, the process can comprise including one or more dose(s) of one or more enzyme(s) during the saccharification and/or the fermentation step. The exogenous enzyme that can be used during the saccharification/fermentation process can include, without limitation, an alpha-amylase, a glucoamylase, a protease, a phytase, a pullulanase, a cellulase, a hemi-cellulase such as a xylanase, a trehalase, or any combination thereof. The enzyme can be purified and/or provided as part of a cocktail. In the process of the present disclosure, a reduced dose or amount of an enzyme can be used in combination with the recombinant yeast host cell of the present disclosure to complete the fermentation.

The process of the present disclosure can include a step of adding a dose (or multiple doses) of an exogenous enzyme (which may be purified) to increase the fermentation yield or allow the yeast to complete the fermentation. In such embodiment, the requirement to add one or more dose(s) can be determined prior to or during fermentation.

For example, the exogenous glucoamylase can be from a Gloeophyllum sp., such as, for example, from Gloeophyllum trabeum. In an embodiment, the exogenous glucoamylase corresponds to Uniprot S7Q4V9 or GenBank Accession Number_007866834. In another embodiment, the exogenous glucoamylase can have the amino acid sequence of SEQ ID NO: 27, be a variant of the amino acid sequence of SEQ ID NO: 27 having glucoamylase activity or be a fragment of the amino acid sequence of SEQ ID NO: 27 having glucoamylase activity (which can, in an embodiment, correspond to a fragment of the amino acid sequence of SEQ ID NO: 27 lacking its signal sequence). For example, the exogenous glucoamylase can be from a Trichoderma sp., such as, for example, from Trichoderma reesii. In an embodiment, the exogenous glucoamylase corresponds to Uniprot GOR866 or GenBank Accession Number_XP_006960925. In another embodiment, the exogenous glucoamylase can have the amino acid sequence of SEQ ID NO: 28, be a variant of the amino acid sequence of SEQ ID NO: 28 having glucoamylase activity or be a fragment of the amino acid sequence of SEQ ID NO: 28 having glucoamylase activity (which can, in an embodiment, correspond to a fragment of the amino acid sequence of SEQ ID NO: 28 lacking its signal sequence). For example, the exogenous glucoamylase can be from a Trametes sp., such as, for example, from Trametes cingulata. In another embodiment, the exogenous glucoamylase can have the amino acid sequence of SEQ ID NO: 29, be a variant of the amino acid sequence of SEQ ID NO: 29 having glucoamylase activity or be a fragment of the amino acid sequence of SEQ ID NO: 29 having glucoamylase activity (which can, in an embodiment, correspond to a fragment of the amino acid sequence of SEQ ID NO: 29 lacking its signal sequence). For example, the exogenous glucoamylase can be from a Athelia sp., such as, for example, from Athelia rolfsil. In an embodiment, the exogenous glucoamylase corresponds to Uniprot Q12596 or GenBank Accession Number_BAA08436. In another embodiment, the exogenous glucoamylase can have the amino acid sequence of SEQ ID NO: 30, be a variant of the amino acid sequence of SEQ ID NO: 30 having glucoamylase activity or be a fragment of the amino acid sequence of SEQ ID NO: 30 having glucoamylase activity (which can, in an embodiment, correspond to a fragment of the amino acid sequence of SEQ ID NO: 30 lacking its signal sequence). For example, the exogenous glucoamylase can be from a Rhizopus sp., such as, for example, from Rhizopus oryzae In an embodiment, the exogenous glucoamylase corresponds to Uniprot P07683 or GenBank Accession Number P07683. In another embodiment, the exogenous glucoamylase can have the amino acid sequence of SEQ ID NO: 31, be a variant of the amino acid sequence of SEQ ID NO: 31 having glucoamylase activity or be a fragment of the amino acid sequence of SEQ ID NO: 31 having glucoamylase activity (which can, in an embodiment, correspond to a fragment of the amino acid sequence of SEQ ID NO: 31 lacking its signal sequence). For example, the exogenous glucoamylase can be from a Aspergillus sp., such as, for example, from Aspergillus oryzae. In an embodiment, the exogenous glucoamylase corresponds to Uniprot P36914 or GenBank Accession Number BAA00841. In another embodiment, the exogenous glucoamylase can have the amino acid sequence of SEQ ID NO: 32, be a variant of the amino acid sequence of SEQ ID NO: 32 having glucoamylase activity or be a fragment of the amino acid sequence of SEQ ID NO: 32 having glucoamylase activity (which can, in an embodiment, correspond to a fragment of the amino acid sequence of SEQ ID NO: 32 lacking its signal sequence). In yet another example, the exogenous heterologous glucoamylase can be from Aspergillus awamori. In an embodiment, the exogenous glucoamylase corresponds to Uniprot Q76L97 or GenBank Accession Number BAD06004. In another embodiment, the exogenous glucoamylase can have the amino acid sequence of SEQ ID NO: 35, be a variant of the amino acid sequence of SEQ ID NO: 35 having glucoamylase activity or be a fragment of the amino acid sequence of SEQ ID NO: 35 having glucoamylase activity (which can, in an embodiment, correspond to a fragment of the amino acid sequence of SEQ ID NO: 35 lacking its signal sequence). In yet another example, the exogenous glucoamylase can be from Aspergillus niger. In an embodiment, the exogenous glucoamylase corresponds to Uniprot Q870G8 or GenBank Accession Number AAP04499. In another embodiment, the exogenous glucoamylase can have the amino acid sequence of SEQ ID NO: 36, be a variant of the amino acid sequence of SEQ ID NO: 36 having glucoamylase activity or be a fragment of the amino acid sequence of SEQ ID NO: 36 having glucoamylase activity (which can, in an embodiment, correspond to a fragment of the amino acid sequence of SEQ ID NO: 36 lacking its signal sequence). For example, the exogenous glucoamylase can be from a Ophiostoma sp., such as, for example, from Ophiostoma floccosum. In an embodiment, the exogenous glucoamylase corresponds to Uniprot Q06SN2 or GenBank Accession Number ABF72529. In another embodiment, the exogenous glucoamylase can have the amino acid sequence of SEQ ID NO: 33, be a variant of the amino acid sequence of SEQ ID NO: 33 having glucoamylase activity or be a fragment of the amino acid sequence of SEQ ID NO: 33 having glucoamylase activity (which can, in an embodiment, correspond to a fragment of the amino acid sequence of SEQ ID NO: 33 lacking its signal sequence). For example, the exogenous glucoamylase can be from a Trichocladium sp., such as, for example, from Trichocladium griseum. In an embodiment, the exogenous glucoamylase corresponds to Uniprot Q12623 or GenBank Accession Number AAA33386. In another embodiment, the exogenous glucoamylase can have the amino acid sequence of SEQ ID NO: 34, be a variant of the amino acid sequence of SEQ ID NO: 34 having glucoamylase activity or be a fragment of the amino acid sequence of SEQ ID NO: 34 having glucoamylase activity (which can, in an embodiment, correspond to a fragment of the amino acid sequence of SEQ ID NO: 34 lacking its signal sequence).

For example, the exogenous alpha-amylase can be from a Rhizomucor sp., such as, for example, from Rhizomucor pusillus. In an embodiment, the exogenous alpha-amylase corresponds to Uniprot M9TI89. In another embodiment, the exogenous alpha-amylase can have the amino acid sequence of SEQ ID NO: 17, be a variant of the amino acid sequence of SEQ ID NO: 17 having alpha-amylase activity or be a fragment of the amino acid sequence of SEQ ID NO: 17 having alpha-amylase activity (which can, in an embodiment, correspond to a fragment of the amino acid sequence of SEQ ID NO: 17 lacking its signal sequence). For example, the exogenous alpha-amylase can be from a Aspergillus sp., such as, for example, from Aspergillus luchuensis. In an embodiment, the exogenous alpha-amylase corresponds to Uniprot A0A146F6W4 or to GenBank Accession Number GAT21778. In another embodiment, the exogenous alpha-amylase can have the amino acid sequence of SEQ ID NO: 18, be a variant of the amino acid sequence of SEQ ID NO: 18 having alpha-amylase activity or be a fragment of the amino acid sequence of SEQ ID NO: 18 having alpha-amylase activity (which can, in an embodiment, correspond to a fragment of the amino acid sequence of SEQ ID NO: 18 lacking its signal sequence). In an embodiment, the exogenous alpha-amylase corresponds to Uniprot O13296 or to GenBank Accession Number BAA22993. In another embodiment, the exogenous alpha-amylase can have the amino acid sequence of SEQ ID NO: 26, be a variant of the amino acid sequence of SEQ ID NO: 26 having alpha-amylase activity or be a fragment of the amino acid sequence of SEQ ID NO: 26 having alpha-amylase activity (which can, in an embodiment, correspond to a fragment of the amino acid sequence of SEQ ID NO: 26 lacking its signal sequence). For example, the exogenous alpha-amylase can be from Aspergillus oryzae. In an embodiment, the exogenous alpha-amylase corresponds to Uniprot Q2UIS5 or to GenBank Accession Number XP_001820542. In another embodiment, the exogenous alpha-amylase can have the amino acid sequence of SEQ ID NO: 19, be a variant of the amino acid sequence of SEQ ID NO: 19 having alpha-amylase activity or be a fragment of the amino acid sequence of SEQ ID NO: 19 having alpha-amylase activity (which can, in an embodiment, correspond to a fragment of the amino acid sequence of SEQ ID NO: 19 lacking its signal sequence). For example, the exogenous alpha-amylase can be from Aspergillus niger. In an embodiment, the exogenous alpha-amylase corresponds to Uniprot A2QTS4 or to GenBank Accession Number XP_001393626. In another embodiment, the exogenous alpha-amylase can have the amino acid sequence of SEQ ID NO: 21, be a variant of the amino acid sequence of SEQ ID NO: 21 having alpha-amylase activity or be a fragment of the amino acid sequence of SEQ ID NO: 21 having alpha-amylase activity (which can, in an embodiment, correspond to a fragment of the amino acid sequence of SEQ ID NO: 21 lacking its signal sequence). In an embodiment, the exogenous alpha-amylase corresponds to Uniprot A2R6F9 or to GenBank Accession Number XP_001397301. In another embodiment, the exogenous alpha-amylase can have the amino acid sequence of SEQ ID NO: 22, be a variant of the amino acid sequence of SEQ ID NO: 22 having alpha-amylase activity or be a fragment of the amino acid sequence of SEQ ID NO: 22 having alpha-amylase activity (which can, in an embodiment, correspond to a fragment of the amino acid sequence of SEQ ID NO: 22 lacking its signal sequence). In an embodiment, the exogenous alpha-amylase corresponds to GenBank Accession Number XP_001395328. In another embodiment, the exogenous alpha-amylase can have the amino acid sequence of SEQ ID NO: 23, be a variant of the amino acid sequence of SEQ ID NO: 23 having alpha-amylase activity or be a fragment of the amino acid sequence of SEQ ID NO: 23 having alpha-amylase activity (which can, in an embodiment, correspond to a fragment of the amino acid sequence of SEQ ID NO: 23 lacking its signal sequence). In an embodiment, the exogenous alpha-amylase corresponds to Uniprot A0A370BQ30 or to GenBank Accession Number RDH15462. In another embodiment, the exogenous alpha-amylase can have the amino acid sequence of SEQ ID NO: 24, be a variant of the amino acid sequence of SEQ ID NO: 24 having alpha-amylase activity or be a fragment of the amino acid sequence of SEQ ID NO: 24 having alpha-amylase activity (which can, in an embodiment, correspond to a fragment of the amino acid sequence of SEQ ID NO: 24 lacking its signal sequence). For example, the exogenous alpha-amylase can be from Aspergillus fischeri. In an embodiment, the exogenous alpha-amylase corresponds to Uniprot A1CYB1 or to GenBank Accession Number XP_001265628. In another embodiment, the exogenous alpha-amylase can have the amino acid sequence of SEQ ID NO: 25, be a variant of the amino acid sequence of SEQ ID NO: 25 having alpha-amylase activity or be a fragment of the amino acid sequence of SEQ ID NO: 25 having alpha-amylase activity (which can, in an embodiment, correspond to a fragment of the amino acid sequence of SEQ ID NO: 25 lacking its signal sequence). For example, the exogenous alpha-amylase can be from a Homo sp., such as, for example, from Homo sapiens. In an embodiment, the exogenous alpha-amylase corresponds to GenBank Accession Number 1B2Y_A. In another embodiment, the exogenous alpha-amylase can have the amino acid sequence of SEQ ID NO: 20, be a variant of the amino acid sequence of SEQ ID NO: 20 having alpha-amylase activity or be a fragment of the amino acid sequence of SEQ ID NO: 20 having alpha-amylase activity (which can, in an embodiment, correspond to a fragment of the amino acid sequence of SEQ ID NO: 20 lacking its signal sequence).

In some embodiments in which the exogenous enzyme is a glucoamylase, it may be beneficial to not over-dose or under-dose the exogenous enzyme. In early- and mid-fermentation, the starch dextrin hydrolysis reaction producing glucose catalyzed by glucoamylase (GA) is substrate-rich. As a result, the hydrolysis reaction rate is primarily controlled by the GA catalyst concentration and the specific reaction frequency (the enzyme turnover frequency) for the GA enzyme protein at the fermentation temperature. At the beginning of fermentation, as the fermentor begins to be filled by liquefact and as either a mash propagation batch or a direct pitch yeast addition are used to inoculate fermentation, the yeast cell concentration in fermentation is at a minimum. Over the first 24-30 hours of fermentation the yeast population increases due to cell growth and division. If GA enzyme is over-dosed in early fermentation, the dextrin hydrolysis rate will significantly exceed the rate of glucose uptake and conversion to ethanol by the yeast, resulting in substantial accumulation of glucose in fermentation (concentrations of glucose greater than 10%-11% w/v). High concentrations of glucose in fermentation can slow yeast growth and fermentation rates due to elevated osmotic pressure applied to the yeast cells; this can result in the inability of the yeast population to convert all of the available glucose in fermentation to ethanol within the available fermentation batch duration. One yeast cellular response to elevated osmotic pressure is to produce higher intracellular concentrations of glycerol to increase the intracellular osmolyte concentration. The diversion of carbon from glucose by the yeast to increased glycerol production, rather than ethanol production, can reduce the ethanol yield achieved in fermentation. On the other hand, if the GA enzyme is under-dosed in fermentation, in early-, mid-, or late-fermentation (depending on how severely the GA enzyme is under-dosed) the rate of glucose uptake and conversion to ethanol by the yeast will exceed the rate of glucose generation from dextrin hydrolysis to such an extent that the glucose concentration will be reduced to nearly zero with a significant concentration of residual starch dextrins remaining to be hydrolyzed and fermented. If the rate of glucose generation at this point is insufficient to supply the rate of energy generation by fermentation required for yeast cell maintenance, then the viable yeast cell population will begin to decrease. Depending on the rate of decrease in yeast cell viability and the fermentation duration remaining until fermentation drop, this may either result in a fermentation batch with a low glucose concentration at drop (≤0.1% w/v), reduced ethanol yield, and elevated concentrations of residual dextrins; or, if the viable yeast population decreases to near zero well before fermentation drop, a fermentation batch with an elevated glucose concentration at drop (>0.1% w/v), normal or elevated concentrations of residual dextrins, and reduced ethanol yield.

In some embodiments, the recombinant yeast host cells described herein reduce the need for (e.g., displace) exogenous enzyme dosing in saccharification/fermentation. As such, a reduced dose of exogenous enzyme can be used in combination with the recombinant yeast host cell of the present disclosure in order to complete the fermentation. The dose used in combination with the recombinant yeast host cell of the present disclosure is considered to be “reduced” because it is lower than a control dose necessary for a control yeast host cell (lacking the ability to hydrolyze starch or dextrin) to complete the fermentation. As used in the context of the present disclosure, a “dose necessary to complete the fermentation” refers to the amount of an exogenous enzyme which is required by a yeast host cell (recombinant or control yeast host cell) to convert the available starch or dextrin in the biomass into glucose, allowing the yeast host cell to convert the glucose into a fermentation product (such as ethanol). As used in the context of the present disclosure, a “reduced dose” does not include the amount of the exogenous enzyme that may be used during the mash propagation step and only refers to the amount of the exogenous enzyme that is used during fermentation. In some specific embodiments, especially when the control yeast host cell is Ethanol Red, the control dose allows achieving, during a permissive fermentation, a fermentation yield of at least 0.415%, w/v per w/w of biomass. In still another specific embodiment, the reduced dose allows achieving, during a permissive fermentation a fermentation yield of at least 0.440%, w/v per w/w of biomass. In some embodiments, the reduced dose of the exogenous enzyme is lower by at least about 50%, 55%, 60%, 61%, 62%, 63%, 64%, 65%, 66%, 67%, 68%, 69%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% on a weight basis when compared to the control dose. In the context of the present disclosure, the term “about”, when used in combination with the % on a weight basis, refers to ±1%. In still another specific embodiment, the exogenous enzyme is lower by at least about 68% on a weight basis when compared to the control dose. In yet another embodiment, the process does not include adding the exogenous enzyme (e.g., enzyme displacement can be 100%). In still another embodiment, the fermentation yield (obtained with the reduced dose) is equal to or higher than the control fermentation yield of the control fermentation (completed by a control yeast cell lacking the ability to hydrolyze starch or dextrin). In still another embodiment, the fermentation yield (obtained with the reduced dose) is substantially similar to the control fermentation yield of the control fermentation (completed by a control yeast cell lacking the ability to hydrolyze starch or dextrin). In the context of the present disclosure, the expression “substantially similar” when used in connection to “the control fermentation yield” refers to a fermentation yield (obtained with a reduced dose and the recombinant yeast host cell) that is ±5% of the control fermentation yield of the control fermentation (completed by a control yeast cell lacking the ability to hydrolyze starch or dextrin).

In some embodiments, the recombinant yeast host cells described herein reduce (e.g., displaces) the need for exogenous enzyme dosing in simultaneous saccharification and fermentation (SSF). In some embodiments, the recombinant yeast host cells described herein alleviate the need for exogenous enzyme dosing in SSF. In some embodiments, the recombinant yeast host cells described herein is less susceptible to temperature and pH stress in SSF processes. As such, in some embodiments of the process, the recombinant yeast host cell of the present disclosure allows for a complete displacement of the exogenous purified enzyme (which can be in some embodiments a glucoamylase) while achieving the same fermentation yield as a corresponding control yeast strain (e.g., lacking ability to saccharify the biomass without addition of the exogenous enzyme) in the presence of a full dose of the exogenous enzyme (which can be in some embodiments a glucoamylase). The process can, in some embodiments, alleviate the need to supplement the hydrolyzed biomass with a exogenous enzyme (which can be in some embodiments a glucoamylase) during the fermentation step.

In some embodiments of the present disclosure, the process can occur at a commercial scale. For example, the process can use a stabilized liquid yeast dose of at least 30 kg, 40 kg, 50 kg, 60 kg, 70 kg, 75 kg, 80 kg, 90 kg, 100 kg, 110 kg, 120 kg, 130 kg, 140 kg, 150 kg, 160 kg, 170 kg, 180 kg, 190 kg, 200 kg, 210 kg or more. In a specific embodiment, the process can use a stabilized liquid yeast dose between 30 and 210 kg, for example between 112.4-168.5 kg. In yet another embodiment, the process can use a stabilized liquid yeast dose between 112.4 kg and 116.4 kg, 112.4 kg and 128.4 kg, 112.4 kg and 132.4 kg, 112.4 kg and 148.5 kg or 112.4 kg and 168.5 kg. In still a further embodiment, the process can use a stabilized liquid yeast dose between 116.4 kg and 128.4 kg, 116.4 kg and 132.4 kg, 116.4 kg and 148.5 kg or 116.4 and 168.5 kg. In still a further embodiment, the process can use a stabilized liquid yeast dose between 128.4 kg and 132.4 kg, 128.4 kg and 148.5 kg or 128.4 kg and 168.5 kg. In still a further embodiment, the process can use a stabilized liquid yeast dose between 132.4 kg and 148.5 kg or 132.4 kg and 168.5 kg. In still a further embodiment, the process can use a stabilized liquid yeast dose between 148.5 kg and 168.5 kg. In yet another embodiment, the process can initially include between 28% and 37% solids, for example between 30% and 35% solids or between 32% and 33% solids. In yet another embodiment, the fermentation duration (at drop) can be at least 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 80, 81, 82, 83, 84, 85, 86, 87, 88, 89 90 hours or more. In yet another embodiment, the fermentation duration can be about 64.3, 65.5, 67, 68.7, 71.4, 74.6, 81.5, 82.3, 82.5, 82.8 hours or more. In commercial application, the process is performed in a fermentor. As it is known in the art, a “fermentor” refers to the apparatus for carrying our fermentation whereas a “fermenter” refers to the actual organism that causes the fermentation. In yet a further embodiment, the working volume of the fermentation reactor/fermentor can be, for example, at least 100 000, 200 000, 300 000, 400 000, 500 000, 600 000, 700 000, 800 000, 900 000, 1 000 000 gallons or more. In yet a further embodiment, the working volume of the fermentation reactor can be, for example, at least 500 000, 525 000, 550 000, 575 000, 600 000, 625 000, 650 000, 675 000, 700 000, 725 000, 750 000, 775 000, 800 000, 825 000, 850 000, 875 000, 900 000 gallons or more. In yet a further embodiment, the working volume of the fermentation reactor/fermentor can be, for example, between 700 000 and 725 000, 700 000 and 750 000, 700 000 and 775 000, 700 000 and 800 000, 700 000 and 825 000, 700 000 and 850 000, 700 000 and 875 000 or 700 000 and 900 000 gallons. In yet a further embodiment, the working volume of the fermentation reactor/fermentor can be, for example, between 725 000 and 750 000, 725 000 and 775 000, 725 000 and 800 000, 725 000 and 825 000, 725 000 and 850 000, 725 000 and 875 000 or 725 000 and 900 000 gallons. In yet a further embodiment, the working volume of the fermentation reactor/fermentor can be, for example, between 750 000 and 775 000, 750 000 and 800 000, 750 000 and 825 000, 750 000 and 850 000, 750 000 and 875 000 or 750 000 and 900 000 gallons. In yet a further embodiment, the working volume of the fermentation reactor/fermentor can be, for example, between 775 000 and 800 000, 775 000 and 825 000, 775 000 and 850 000, 775 000 and 875 000 or 775 000 and 900 000 gallons. In yet a further embodiment, the working volume of the fermentation reactor/fermentor can be, for example, between 800 000 and 825 000, 800 000 and 850 000, 800 000 and 875 000 or 800 000 and 900 000 gallons. In yet a further embodiment, the working volume of the fermentation reactor/fermentor can be, for example, between 825 000 and 850 000, 825 000 and 875 000 or 825 000 and 900 000 gallons. In yet a further embodiment, the working volume of the fermentation reactor/fermentor can be, for example, between 850 000 and 875 000 or 850 000 and 900 000 gallons. In yet a further embodiment, the working volume of the fermentation reactor/fermentor can be, for example, between 875 000 and 900 000 gallons. In still a further example, especially when the fermentation is a batch fermentation, the fermentor fill duration can be at least 2, 3, 4, 5, 6, 7, 8, 9, 10, 11, 12, 13, 14, 15, 16, 17, 18, 19, 20 hours or more. In some embodiments, the fermentor fill duration can be between 5 and 20 hours.

### Yeast Products and Compositions

The recombinant yeast host cells of the present disclosure can be used in the preparation of a yeast composition (e.g., a composition comprising the recombinant yeast host cell) comprising the heterologous polypeptide having starch or dextrin hydrolase activity. The yeast compositions and products can be provided in a liquid, semi-liquid or dry form.

A yeast composition refers to a composition comprising the recombinant yeast host cell of the present disclosure (which may be, in some embodiments, a viable recombinant yeast host cell) as well as the heterologous polypeptide having starch or dextrin hydrolase activity. The process for providing a yeast composition comprises providing a propagated the recombinant yeast host cell and removing, at least one component of the mixture obtained after propagation to provide the yeast composition. This component can be, without limitation, water, amino acids, peptides and proteins, nucleic acid residues and nucleic acid molecules, cellular debris, fermentation products, etc. In an embodiment, the process comprises substantially isolating the propagated recombinant yeast host cells from the components of the propagation medium. As used in the context of the present disclosure, the expression “substantially isolating” refers to the removal of the majority of the components of the propagation medium from the propagated recombinant yeast host cells. In some embodiments, “substantially isolating” refers to concentrating the propagated recombinant yeast host cell to at least 5, 10, 15, 20, 25, 30, 35, 45% or more when compared to the concentration of the recombinant yeast host cell prior to the isolation. In order to provide the yeast composition, the propagated recombinant yeast host cells can be centrifuged (and the resulting cellular pellet comprising the propagated recombinant yeast host cells can optionally be washed), filtered and/or dried (optionally using a vacuum-drying technique). The isolated recombinant yeast host cells can then be formulated in a yeast composition. The yeast composition can be provided in an active or a semi-active form. The yeast composition can be provided in a liquid, semi-solid or dry form. In an embodiment, the yeast composition can be provided in the form of a cream yeast. In some embodiments, the process also include propagating the recombinant yeast host cell prior to the removal step. The yeast composition can be optionally stored prior to the fermentation phase. In such embodiment, the yeast composition can include, for example, one or more stabilizers or preservatives and, in some embodiment, an unfermentable carbon source (such as trehalose for example).

In some embodiments, the recombinant yeast host cell or the yeast composition obtained therefrom can be provided in a composition in combination with starch. Such composition can include additional exogenous enzyme(s) which may be used during the saccharification and/or fermentation steps.

The present invention will be more readily understood by referring to the following examples which are given to illustrate the invention rather than to limit its scope.

### EXAMPLE I—HETEROLOGOUS STARCH DIGESTING GLUCOAMYLASE IN RECOMBINANT YEAST CELLS

TABLE 1
Genotypes of the strains used in the examples. All the recombinant
strains were derived from M2390 and expressed the recombinant
enzyme under transcriptional control of the constitutive
tef2p promoter and idp1t terminator.

| Strain name | Expressed enzyme | Enzyme description |
|---|---|---|
| M2390 | None-wild-type Saccharomyces cerevisiae strain | |
| M17199 | MP1152 | Glucoamylase from |
| | (SEQ ID NO: 9) | Saccharomycopsis fibuligera |
| | | associated with the signal |
| | | sequence of Saccharomyces |
| | | cerevisiae alpha-mating |
| | | factor 1 |
| M15621 | SEQ ID NO: 6 | Glucoamylase from Rasamsonia |
| | | emersonii associated with |
| | | its native signal sequence |
| M23176 and | MP1262 | Glucoamylase from Rasamsonia |
| M23177* | (SEQ ID NO: 1) | emersonii associated with |
| | | the signal sequence of |
| | | Saccharomyces cerevisiae |
| | | alpha-mating factor 1 |

*M23176 and M23177 are two different isolates from the same transformation

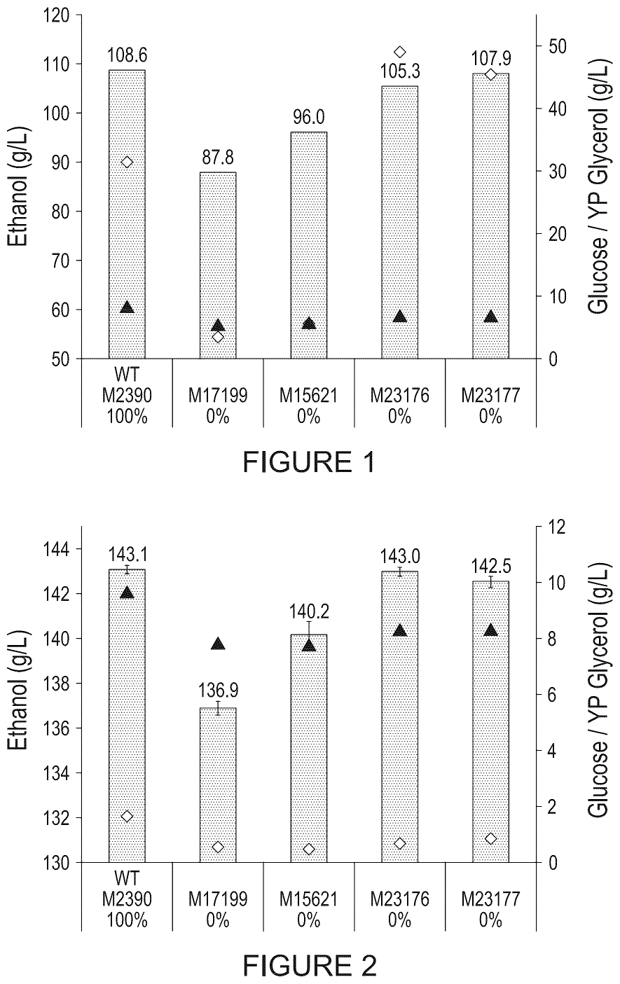

Permissive corn mash fermentation data. Permissive fermentation conditions were conducted as follows: 32.4% total solids, 300 ppm urea, 33° C. (1-48 hours), exogenous glucoamylase GA enzyme inclusion as listed under each bar of the figures. FIG. 1 illustrates the surprising result of MP1262 secreting strains (M23176 and M23177) showing performance parity at 0% exogenous glucoamylase inclusion to M2390 dosed with a full 100% dose of exogenous enzyme. Without wishing to be bound to theory, the fact that the ethanol titers show parity for isolates M23176/M23177 with M2390 suggests that the expression of the heterologous MP1262 does not seem to reduce strain fermentative performance.

FIG. 2 further illustrates that the signal peptide optimized MP1262 secreting strains can successfully finish fermentation with 100% exogenous enzyme displacement, achieving the same titers as M2390 dosed with a full 100% GA dose.

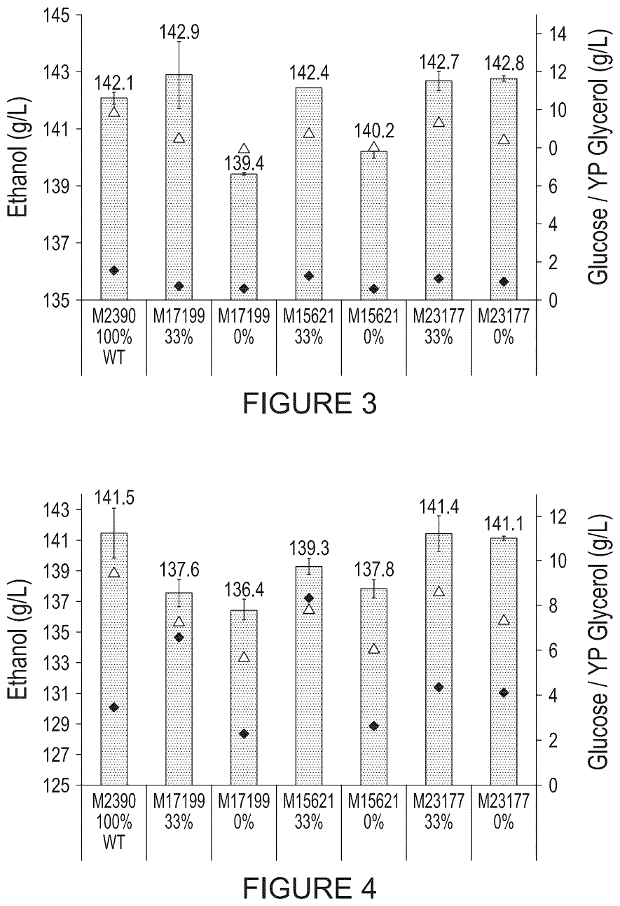

In FIG. 3, four strains were compared side by side at either 33% or 0% exogenous enzyme inclusion. The reduction in fermentation performance going from 33% to 0% enzyme inclusion for M17199 and M15621 can be attributed to insufficient heterologous GA secretion in the conditions tested. Strain M23177 did not exhibit a reduction in in fermentation performance in the absence of the exogenous enzyme.

Non-permissive corn mash fermentation data. The fermentations were conducted according to the following conditions: 32.4% total solids, no urea, 34° C. (1-48 hours) or 36° C. (1-48 hours, for heat-treatment challenge only), 0.38% w/v lactic acid added 20 hours into the fermentation (for lactic acid challenge only), exogenous glucoamylase (GA) enzyme inclusion as listed under each bar of the figures.

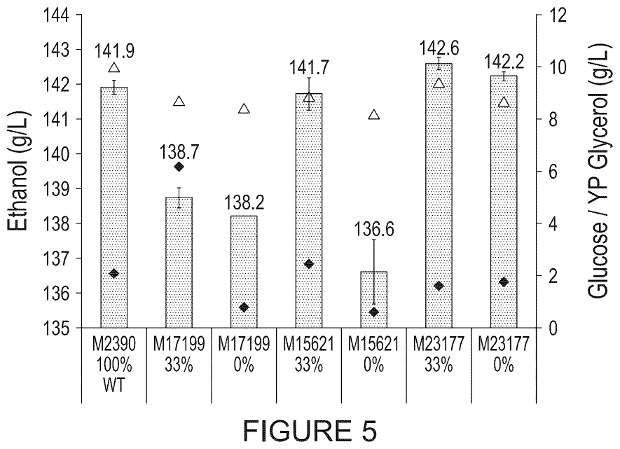

As shown in FIGS. 4 and 5, strain M23177 did not exhibit a reduction in in fermentation performance in the absence of the exogenous enzyme during non-permissive fermentation (FIG. 4 shows the results associated with a lactic acid challenge and FIG. 5 shows the results associated with a heat treatment).

### EXAMPLE II—ENZYME DISPLACEMENT

The fermentation performances of two recombinant Saccharomyces cerevisiae host cell types have been compared to a non-engineered yeast strain at Production Facility A. Yeast strain M23541 expresses a secreted form of a Rasamsonia emersonii glucoamylase (having the amino acid sequence of SEQ ID NO: 1). Yeast strain M15419 (corresponding to the commercial product TransFerm®RB3) expressed a secreted form of a Saccharomycopsis fibuligera (glucoamylase having the amino acid sequence of SEQ ID NO: 37). Yeast strains M23541 and M15419 both bear additional “glycerol reduction background modifications” described in WO2011140386, WO2012138942 and WO2020100069 (all incorporated herewith in their entirety) and allowing the reduction of production of glycerol. Table 2 summarizes the progression of the testing, and the conditions present during each testing phase, from use of conventional yeast. Table 3 summarizes the average (mean) measurements for all fermentation batches within trial phases, including average slurry total solids used to produce liquefact filling fermentation batches, average component concentrations (DP4+, DP3, DP2, glucose, glycerol, ethanol, total residual sugars) in fermentation beer soluble fraction (supernatant) over the time course of fermentation batches, average ethanol yield per slurry total solids over the time course of fermentation batches (ethanol concentration in fermentation beer soluble fraction normalized to batch slurry total solids), and average fermentation duration.

TABLE 2
Conditions during progression of testing at Production Facility A, including: average slurry total solids,
average fermentation duration, yeast type added in mash propagation, yeast dose used in mash propagation,
exogenous GA cocktail dose used in mash propagation, exogenous GA cocktail dose used in fermentation,
displacement of exogenous GA cocktail dose in fermentation relative to dose used with conventional yeast
(yeast not expressing starch- or dextrin-degrading enzymes), exogenous GA cocktail type used in mash
propagation and fermentation, and exogenous alpha-amylase (AA) type used in mashing process.

| | | | | | Average | |
|---|---|---|---|---|---|---|
| | | | | Average | Fermentation | |
| | | | | Slurry | Duration | |
| | | | | Total Solids | for Phase | |
| | | Number of | | for Phase | (at Drop, | |
| Week | Phase | Batches | Phase Detail | (% w/w) | hours) | Yeast Type |
| Week 1 | A | 16 | Conventional Yeast, 32% | 32.01 | 67.0 | Ethanol Red; |
| | | | w/w Slurry Solids Target, | | | Conventional |
| | | | 100 gal Exogenous GA | | | Active Dry |
| | | | Cocktail in Fermentation, | | | Yeast Type B |
| | | | 0.75 gal Exogenous GA | | | |
| | | | Cocktail in Propagation, | | | |
| | | | 40 kg Active Dry Yeast Dose | | | |
| Week 1-2 | B | 11 | M15419, 32% w/w Slurry | 31.86 | 65.5 | M15419 |
| | | | Solids Target, 51 gal | | | |
| | | | Exogenous GA Cocktail in | | | |
| | | | Fermentation, 0.75 gal | | | |
| | | | Exogenous GA Cocktail in | | | |
| | | | Propagation, 112.4 kg | | | |
| | | | Stabilized Liquid Yeast Dose | | | |
| Week 2 | C | 15 | M15419, 33% w/w Slurry | 32.95 | 64.3 | M15419 |
| | | | Solids Target, 51 gal | | | |
| | | | Exogenous GA Cocktail in | | | |
| | | | Fermentation, 0.75 gal | | | |
| | | | Exogenous GA Cocktail in | | | |
| | | | Propagation, 112.4 kg | | | |
| | | | Stabilized Liquid Yeast Dose | | | |
| Week 30 | D | 3 | M23541, 33% w/w Slurry | 32.99 | 82.8 | M23541 |
| | | | Solids Target, 17 gal | | | |
| | | | Exogenous GA Cocktail in | | | |
| | | | Fermentation, 0.5 gal | | | |
| | | | Exogenous GA Cocktail in | | | |
| | | | Propagation, 168.5 kg | | | |
| | | | Stabilized Liquid Yeast Dose | | | |
| Week 30 | E | 1 | M23541, 33% w/w Slurry | 32.77 | 82.3 | M23541 |
| | | | Solids Target, 10 gal | | | |
| | | | Exogenous GA Cocktail in | | | |
| | | | Fermentation, 0.5 gal | | | |
| | | | Exogenous GA Cocktail in | | | |
| | | | Propagation, 168.5 kg | | | |
| | | | Stabilized Liquid Yeast Dose | | | |
| Weeks 30-31 | F | 11 | M23541, 33% w/w Slurry | 32.97 | 83.3 | M23541 |
| | | | Solids Target, 10 gal | | | |
| | | | Exogenous GA Cocktail in | | | |
| | | | Fermentation, No | | | |
| | | | Exogenous GA Cocktail in | | | |
| | | | Propagation, 168.5 kg | | | |
| | | | Stabilized Liquid Yeast Dose | | | |
| Week 31 | G | 3 | M23541, 33% w/w Slurry | 33.01 | 82.5 | M23541 |
| | | | Solids Target, 10 gal | | | |
| | | | Exogenous GA Cocktail in | | | |
| | | | Fermentation, No | | | |
| | | | Exogenous GA Cocktail in | | | |
| | | | Propagation, 148.5 kg | | | |
| | | | Stabilized Liquid Yeast Dose | | | |
| Week 31 | H | 1 | M23541, 33% w/w Slurry | 33.03 | 81.5 | M23541 |
| | | | Solids Target, No | | | |
| | | | Exogenous GA Cocktail in | | | |
| | | | Fermentation, No | | | |
| | | | Exogenous GA Cocktail in | | | |
| | | | Propagation, 148.5 kg | | | |
| | | | Stabilized Liquid Yeast Dose | | | |
| Weeks 31-32 | I | 17 | M23541, 33% w/w Slurry | 33.00 | 74.6 | M23541 |
| | | | Solids Target, 10 gal | | | |
| | | | Exogenous GA Cocktail in | | | |
| | | | Fermentation, No Exogenous | | | |
| | | | GA Cocktail in Propagation, | | | |
| | | | 148.5 kg Stabilized Liquid | | | |
| | | | Yeast Dose, Shorter | | | |
| | | | Fermentation Durations | | | |
| Weeks 32-33 | J | 28 | M23541, 33% w/w Slurry | 32.99 | 71.4 | M23541 |
| | | | Solids Target, 10 gal | | | |
| | | | Exogenous GA Cocktail in | | | |
| | | | Fermentation, No Exogenous | | | |
| | | | GA Cocktail in Propagation, | | | |
| | | | 128.4 kg Stabilized Liquid | | | |
| | | | Yeast Dose, Shorter | | | |
| | | | Fermentation Durations | | | |
| Week 33 | K | 5 | M23541, 33% w/w Slurry | 33.03 | 68.7 | M23541 |
| | | | Solids Target, 10 gal | | | |
| | | | Exogenous GA Cocktail in | | | |
| | | | Fermentation, No Exogenous | | | |
| | | | GA Cocktail in Propagation, | | | |
| | | | 116.4 kg Stabilized Liquid | | | |
| | | | Yeast Dose, Shorter | | | |
| | | | Fermentation Durations | | | |

| | Yeast Dose | | | | | |
|---|---|---|---|---|---|---|
| | in Mash | | | | | |
| | Propagation | | | Displacement | | Exogenous |
| | (ADY = | Total Mash | Total | of Exogenous | Exogenous | Alpha-Amylase |
| | Active Dry | Propagation | Fermentation | GA Cocktail | GA Cocktail | (AA) Cocktail |
| | Yeast, SLY = | Exogenous | Exogenous | Dose in | Type in | Type Used in |
| | Stabilized | GA Cocktail | GA Cocktail | Fermentation | Propagation & | Mashing |
| Week | Liquid Yeast) | Dose (gal) | Dose (gal) | (%) | Fermentation | Process |
| Week 1 | 40 kg | 0.75 | 100 | 0.0% | Third-Party | Third-Party |
| | ADY | | | | Exogenous | Exogenous AA |
| | | | | | GA Cocktail | Cocktail |
| | | | | | Type A | Type A |
| Week 1-2 | 112.4 kg | 0.75 | 51 | 49.0% | Third-Party | Third-Party |
| | SLY | | | | Exogenous | Exogenous AA |
| | | | | | GA Cocktail | Cocktail |
| | | | | | Type A | Type A |
| Week 2 | 112.4 kg | 0.75 | 51 | 49.0% | Third-Party | Third-Party |
| | SLY | | | | Exogenous | Exogenous AA |
| | | | | | GA Cocktail | Cocktail |
| | | | | | Type A | Type A |
| Week 30 | 168.5 kg | 0.50 | 17 | 83.0% | Third-Party | Third-Party |
| | SLY | | | | Exogenous | Exogenous AA |
| | | | | | GA Cocktail | Cocktail |
| | | | | | Type B | Type B |
| Week 30 | 168.5 kg | 0.50 | 10 | 90.0% | Third-Party | Third-Party |
| | SLY | | | | Exogenous | Exogenous AA |
| | | | | | GA Cocktail | Cocktail |
| | | | | | Type B | Type B |
| Weeks 30-31 | 168.5 kg | 0.00 | 10 | 90.0% | Third-Party | Third-Party |
| | SLY | | | | Exogenous | Exogenous AA |
| | | | | | GA Cocktail | Cocktail |
| | | | | | Type B | Type B |
| Week 31 | 148.5 kg | 0.00 | 10 | 90.0% | Third-Party | Third-Party |
| | SLY | | | | Exogenous | Exogenous AA |
| | | | | | GA Cocktail | Cocktail |
| | | | | | Type B | Type B |
| Week 31 | 148.5 kg | 0.00 | 0 | 100.0% | Third-Party | Third-Party |
| | SLY | | | | Exogenous | Exogenous AA |
| | | | | | GA Cocktail | Cocktail |
| | | | | | Type B | Type B |
| Weeks 31-32 | 148.5 kg | 0.00 | 10 | 90.0% | Third-Party | Third-Party |
| | SLY | | | | Exogenous | Exogenous AA |
| | | | | | GA Cocktail | Cocktail |
| | | | | | Type B | Type B |
| Weeks 32-33 | 128.4 kg | 0.00 | 10 | 90.0% | Third-Party | Third-Party |
| | SLY | | | | Exogenous | Exogenous AA |
| | | | | | GA Cocktail | Cocktail |
| | | | | | Type B | Type B |
| Week 33 | 116.4 kg | 0.00 | 10 | 90.0% | Third-Party | Third-Party |
| | SLY | | | | Exogenous | Exogenous AA |
| | | | | | GA Cocktail | Cocktail |
| | | | | | Type B | Type B |

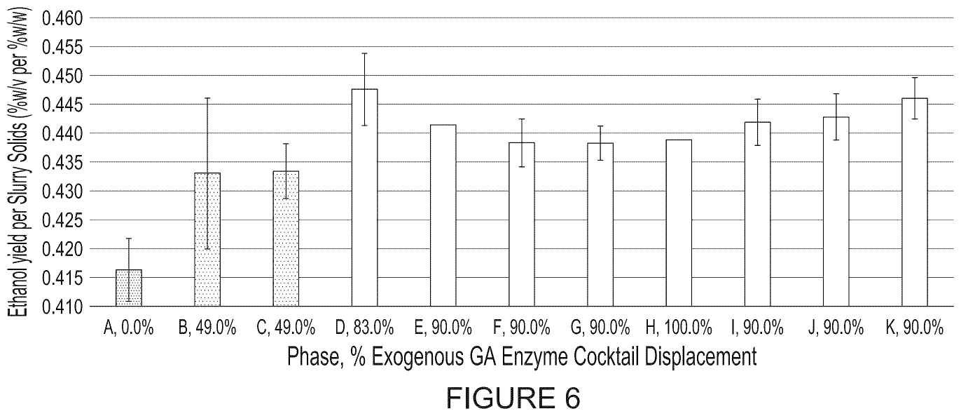

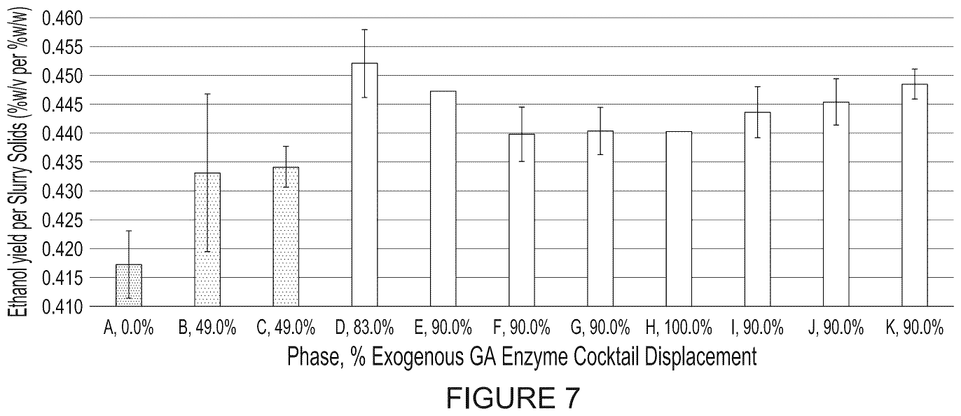

TABLE 3
Average (mean) measurements for all fermentation batches within trial phases at Production Facility A, including average slurry
total solids used to produce liquefact filling fermentation batches, average component concentrations (DP4+, DP3, DP2, glucose,
glycerol, ethanol, total residual sugars) in fermentation beer soluble fraction (supernatant) over the time course of fermentation
batches, average ethanol yield per slurry total solids over the time course of fermentation batches (ethanol concentration in
fermentation beer soluble fraction normalized to batch slurry total solids), and average fermentation duration.

| | | Phase | | | | | | | | | | |
|---|---|---|---|---|---|---|---|---|---|---|---|---|
| | | A | B | C | D | E | F | G | H | I | J | K |
| Displacement of Exogenous GA | | 0.0% | 49.0% | 49.0% | 83.0% | 90.0% | 90.0% | 90.0% | 100.0% | 90.0% | 90.0% | 90.0% |
| Enzyme Cocktail Dose in | | | | | | | | | | | | |
| Fermentation (%) | | | | | | | | | | | | |
| Measurement | Fermentation | Mean | Mean | Mean | Mean | Mean | Mean | Mean | Mean | Mean | Mean | Mean |
| | Sample Time | | | | | | | | | | | |
| | Point (h) | | | | | | | | | | | |
| Slurry Total | Average During | 32.01 | 31.86 | 32.95 | 32.99 | 32.77 | 32.97 | 33.01 | 33.03 | 33.00 | 32.99 | 33.03 |
| Solids (% w/w) | Fermentor Fill | | | | | | | | | | | |
| Ethanol (% w/v) | 10 h | 2.287 | 2.118 | 2.017 | 2.408 | 2.247 | 2.552 | 2.242 | 2.242 | 2.360 | 2.327 | 2.071 |
| | 18 h | 6.166 | 5.889 | 5.741 | 6.360 | 6.295 | 6.820 | 6.541 | 6.210 | 6.500 | 6.328 | 5.750 |
| | 25 h | 8.746 | 8.398 | 8.382 | 9.021 | 8.924 | 9.579 | 9.396 | 9.095 | 9.223 | 8.986 | 8.537 |
| | 39 h | 12.422 | 11.899 | 11.840 | 12.369 | 12.402 | 13.261 | 13.037 | 12.536 | 12.676 | 12.522 | 12.036 |
| | 52 h | 13.214 | 13.551 | 13.770 | 14.456 | 13.741 | 14.436 | 14.457 | 14.065 | 14.418 | 14.410 | 14.140 |
| | 60 h | 13.329 | 13.789 | 14.285 | 14.769 | 14.468 | 14.452 | 14.465 | 14.495 | 14.583 | 14.607 | 14.731 |
| | Fermentation | 13.358 | 13.790 | 14.306 | 14.914 | 14.656 | 14.500 | 14.537 | 14.544 | 14.643 | 14.693 | 14.812 |
| | Drop | | | | | | | | | | | |
| Ethanol Yield | 10 h | 0.0714 | 0.0667 | 0.0612 | 0.0730 | 0.0686 | 0.0774 | 0.0679 | 0.0679 | 0.0715 | 0.0706 | 0.0627 |
| (Batch Ethanol | 18 h | 0.1926 | 0.1850 | 0.1742 | 0.1928 | 0.1921 | 0.2069 | 0.1982 | 0.1880 | 0.1970 | 0.1918 | 0.1741 |
| Concentration | 25 h | 0.2732 | 0.2637 | 0.2544 | 0.2734 | 0.2723 | 0.2905 | 0.2846 | 0.2753 | 0.2794 | 0.2724 | 0.2585 |
| Normalized to | 39 h | 0.3880 | 0.3737 | 0.3593 | 0.3749 | 0.3785 | 0.4022 | 0.3949 | 0.3795 | 0.3841 | 0.3796 | 0.3644 |
| Batch Slurry | 52 h | 0.4128 | 0.4256 | 0.4179 | 0.4382 | 0.4193 | 0.4378 | 0.4380 | 0.4258 | 0.4368 | 0.4368 | 0.4281 |
| Total Solids, | 60 h | 0.4164 | 0.4331 | 0.4335 | 0.4476 | 0.4415 | 0.4383 | 0.4382 | 0.4388 | 0.4419 | 0.4428 | 0.4460 |
| % w/v Ethanol | Fermentation | 0.4173 | 0.4331 | 0.4341 | 0.4520 | 0.4473 | 0.4398 | 0.4404 | 0.4403 | 0.4437 | 0.4454 | 0.4485 |
| at Time Point | Drop | | | | | | | | | | | |
| per % w/w | | | | | | | | | | | | |
| Slurry Total | | | | | | | | | | | | |
| Solids) | | | | | | | | | | | | |
| Fermentation | Start of | 67.0 | 65.5 | 64.3 | 82.8 | 82.3 | 83.3 | 82.5 | 81.5 | 74.6 | 71.4 | 68.7 |
| Duration (h) | Fermentor Fill | | | | | | | | | | | |
| | to Fermentation | | | | | | | | | | | |
| | Drop | | | | | | | | | | | |

Whereas the conventional yeast not engineered to secrete glucoamylase required a 100 gal dose of an exogenous GA enzyme cocktail in fermentation to facilitate starch dextrin hydrolysis to glucose and fermentation to ethanol, and yeast strain M15419 required a 51 gal dose of the exogenous GA enzyme cocktail in fermentation (a 49.0% displacement of exogenous GA cocktail dosage relative to conventional yeast), strain M23541 required between zero exogenous GA in mash propagation and fermentation to 17 gal of exogenous GA cocktail in fermentation (between complete, 100.0% displacement of the exogenous GA cocktail in mash propagation and fermentation, and 83.0% displacement of the exogenous GA cocktail in fermentation) (Table 2). As the total final fermentation durations varied between yeast strain types and conditions, ethanol yield data is presented here for all conditions after both 60 h of fermentation (FIG. 6), and at the end of fermentation (fermentation drop, FIG. 7).

At 60 h fermentation duration (FIG. 6), use of strain M23541 allowed for statistically significantly higher ethanol yields (95% confidence level) relative to conventional yeast used at 100 gal of exogenous GA cocktail at all M23541 yeast doses and exogenous GA dose conditions shown (between 116.4 kg and 168.5 kg of M23541 stabilized liquid yeast used to inoculate mash propagation, and between zero exogenous GA enzyme cocktail used in mash propagation and fermentation and 17 gal of exogenous GA cocktail used in fermentation with 0.5 gal of exogenous GA enzyme cocktail used in mash propagation). At 60 h fermentation duration (FIG. 6), use of strain M23541 allowed for statistically equivalent or higher ethanol yields (95% confidence level) relative to strain M15419 used at 51 gal of exogenous GA enzyme cocktail at all M23541 yeast doses and exogenous GA cocktail dose conditions shown. Use of M23541 at doses of 10 gal exogenous GA enzyme cocktail in fermentation and at zero gallons of exogenous GA enzyme cocktailing fermentation did not result in statistically significantly different ethanol yields from M23541 used at a 17 gal exogenous GA enzyme cocktail dosed in fermentation.

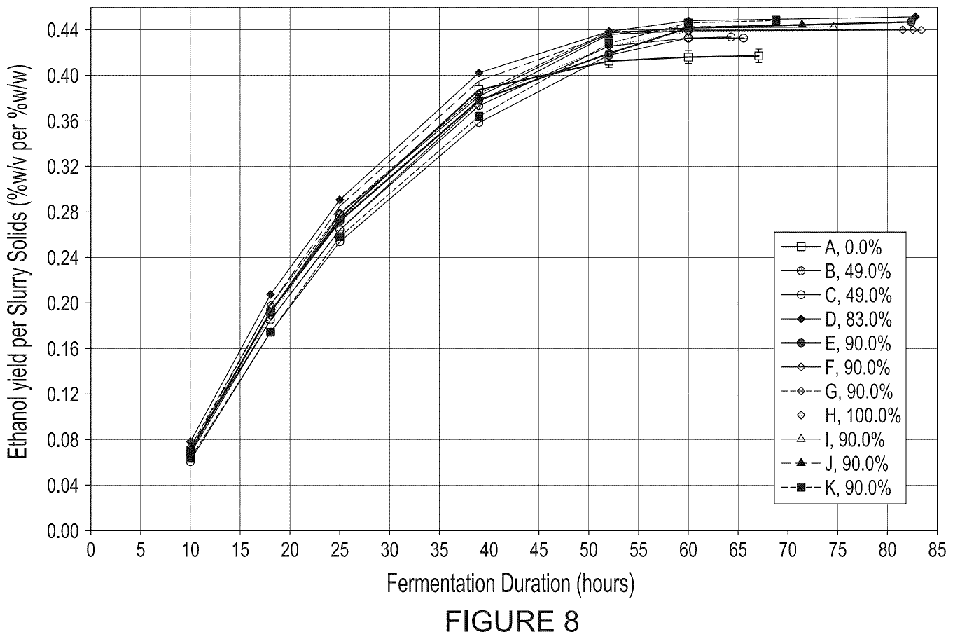

By the end of fermentation (fermentation drop, FIG. 7), use of strain M23541 allowed for statistically significantly higher ethanol yields (95% confidence level) relative to conventional yeast used at 100 gal of exogenous GA cocktail at all M23541 yeast doses and exogenous GA dose conditions shown (between 116.4 kg and 168.5 kg of M23541 stabilized liquid yeast used to inoculate mash propagation, and between zero gallons of exogenous GA enzyme cocktail used in mash propagation and fermentation and 17 gal of exogenous GA enzyme cocktail used in fermentation with 0.5 gal of exogenous GA enzyme cocktail used in mash propagation). By the end of fermentation (FIG. 7), use of strain M23541 allowed for statistically equivalent or higher ethanol yields (95% confidence level) relative to strain M15419 used at 51 gal of exogenous GA cocktail at all M23541 yeast doses and exogenous GA enzyme cocktail dose conditions shown. Use of M23541 at doses of 10 gal exogenous GA enzyme cocktail in fermentation and at zero gallons of exogenous GA enzyme cocktail in fermentation did not result in statistically significantly different ethanol yields from M23541 used at a 17 gal exogenous GA enzyme cocktail dosed in fermentation. The evolution of ethanol yield by strain and condition tested over the course of fermentation duration is summarized in FIG. 8.

By 60 h in fermentation, the extent of hydrolysis and conversion of soluble dextrins with use of strain M23541 at all yeast doses and exogenous GA dose conditions shown (between 116.4 kg and 168.5 kg of M23541 stabilized liquid yeast used to inoculate mash propagation, and between zero gallons of exogenous GA enzyme cocktail used in mash propagation and fermentation and 17 gal of exogenous GA enzyme cocktail used in fermentation with 0.5 gal of exogenous GA enzyme cocktail used in mash propagation) was equivalent to or better than (lower than) with use of both conventional yeast (Ethanol Red and a yeast product of an equivalent nature not engineered to express glucoamylase or other starch- or dextrin-degrading enzymes) at 100 gal of exogenous GA cocktail and use of strain M15419 at 51 gal of exogenous GA cocktail, as indicated by total residual sugar concentrations at this time.

### EXAMPLE III—CORN FERMENTATION USING DRY YEAST SAMPLES

Permissive corn mash fermentation. Permissive fermentation conditions were conducted as follows: 31.55% total solids, 612 ppm urea, 32° C. (0-52 hours), exogenous glucoamylase GA enzyme inclusion as listed under each bar of the figures. The “100%” enzyme dose is equivalent to 0.6 AGU/gTS. Yeast dosing was carried out through direct pitch (0.05 g dry cell weight/L inoculum) from dry yeast samples rehydrated in sterile water at ambient temperature for 30 minutes. The fermentations were carried out using 10 mL scintillation vials with a total sample size of 3 grams.

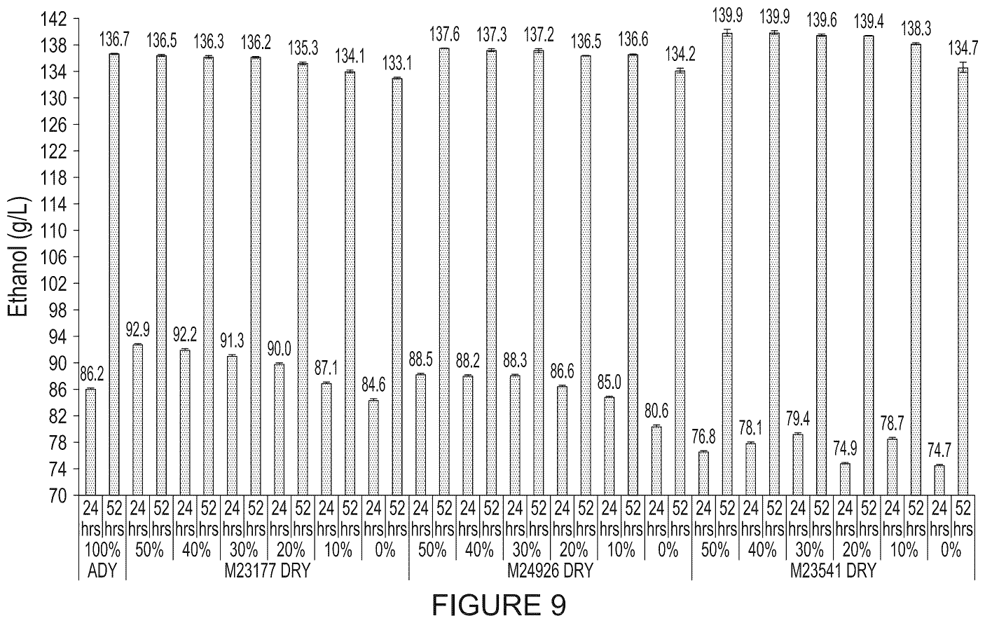

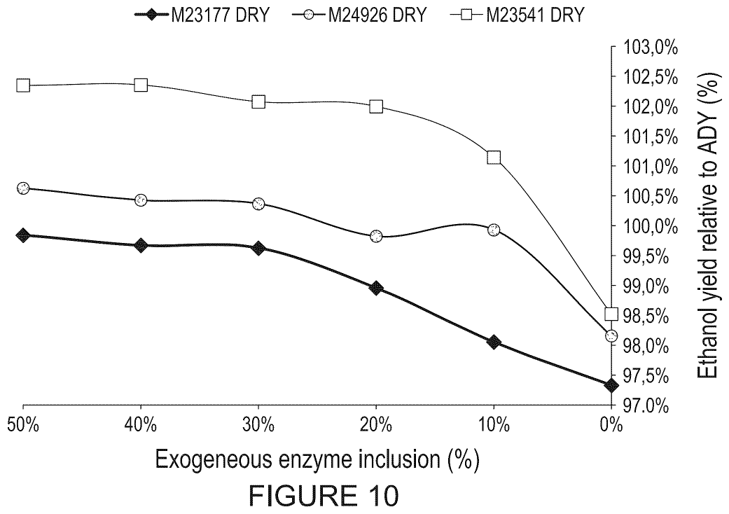

The fermentation performance of three distinct yeast strains has been determined. Once reconstituted, the dry yeasts samples comprising strains M23177 (described in Example I), M24926 (expressing the R. emersonii glucoamylase with the alpha-mating factor signal sequence like strain M23177 and bearing an additional “trehalose reduction” genetic modification described in U.S. Pat. No. 10,570,421 and incorporated herewith in their entirety) and M23541 (expressing the R. emersonii glucoamylase with the alpha-mating factor signal sequence like strain M23177 and bearing additional “glycerol reduction background modifications” refers the genetic modifications described in WO2011140386, WO2012138942 and WO2020100069 allowing the reduction of production of glycerol, all incorporated herewith in their entirety) were shown to produce ethanol even when a lower dose of exogenous glucoamylase was used (FIG. 9). In FIG. 10, the results obtained in FIG. 9 (at 52 hours) were plotted as relative ethanol yield compared to the result obtained with the conventional strain Ethanol Red (provided in an active dried form or ADY).

While the invention has been described in connection with specific embodiments thereof, it will be understood that the scope of the claims should not be limited by the preferred embodiments set forth in the examples, but should be given the broadest interpretation consistent with the description as a whole.

What is claimed is:

1. A process for fermenting a biomass into a fermentation product, the process comprises contacting the biomass with a recombinant yeast host cell and a reduced dose of an exogenous enzyme, under a condition that allows the conversion of at least a part of the biomass into the fermentation product, wherein:
the recombinant yeast host cell has a heterologous nucleic acid molecule encoding a heterologous polypeptide having starch or dextrin hydrolase activity, wherein the heterologous nucleic acid molecule comprises a first polynucleotide encoding a heterologous signal sequence; and a second polynucleotide encoding a heterologous polypeptide having starch or dextrin hydrolase activity;
the first polynucleotide molecule is operatively associated with the second polynucleotide molecule;
the exogenous enzyme is a starch or dextrin hydrolase; and
the reduced dose of the exogenous enzyme is lower than a control dose necessary for a control yeast host cell lacking the ability to hydrolyze starch or dextrin to complete a corresponding control fermentation.

2. The process of claim 1, wherein the control yeast host cell lacks the heterologous nucleic acid molecule.

3. The process of claim 1, wherein the heterologous polypeptide having starch or dextrin hydrolase activity is an heterologous polypeptide having glucoamylase activity.

4. The process of claim 3, wherein the polypeptide having glucoamylase activity has the amino acid sequence of SEQ ID NO: 3 or 13, is a variant of the amino acid sequence of SEQ ID NO: 3, 13 having glucoamylase activity, or is a fragment of the amino acid sequence of SEQ ID NO: 3 or 13 having glucoamylase activity.

5. The process of claim 1, wherein the heterologous signal sequence has the amino acid sequence of SEQ ID NO: 5, is a variant of the amino acid sequence of SEQ ID NO: 5 having signal sequence activity, or is a fragment of the amino acid sequence of SEQ ID NO: 5 having signal sequence activity.

6. The process of claim 1, wherein the heterologous nucleic acid molecule encodes the heterologous polypeptide having the amino acid sequence of SEQ ID NO: 1 or 11, a variant of the amino acid sequence of SEQ ID NO: 1 or 11 having glucoamylase activity, or a fragment of the amino acid sequence of SEQ ID NO: 1 or 11 having glucoamylase activity.

7. The process of claim 1, wherein the heterologous nucleic acid molecule further comprises a third polynucleotide comprising a heterologous promoter operatively associated with the first polynucleotide and the second polynucleotide allowing the expression of the heterologous polypeptide having starch or dextrin hydrolase activity.

8. The process of claim 7, wherein the heterologous promoter is capable of allowing the expression of the heterologous polypeptide having starch or dextrin hydrolase activity during propagation.

9. The process of claim 8, wherein the heterologous polypeptide having starch or dextrin activity is a secreted polypeptide.

10. The process of claim 8, wherein the heterologous polypeptide having starch or dextrin activity is a membrane-associated polypeptide.

11. The process of claim 10, wherein the membrane-associated polypeptide is a tethered polypeptide.

12. The process of claim 1, wherein the recombinant yeast host cell comprises a further heterologous nucleic acid molecule encoding a heterologous alpha-amylase and/or a heterologous glucoamylase.

13. The process of claim 12, wherein the heterologous alpha-amylase has the amino acid sequence of any one of SEQ ID NO: 17 to 26, is a variant of the amino acid sequence of any one of SEQ ID NO: 17 to 26 having alpha-amylase activity or is a fragment of the amino acid sequence of any one of SEQ ID NO: 17 to 26 having alpha-amylase activity.

14. The process of claim 12, wherein the heterologous glucoamylase has the amino acid sequence of any one of SEQ ID NO: 27 to 36, a variant of the amino acid sequence of any one of SEQ ID NO: 27 to 36 having glucoamylase activity or a fragment of the amino acid sequence of any one of SEQ ID NO: 27 to 36 having glucoamylase activity.

15. The process of claim 1, wherein the recombinant yeast host cell is from the genus Saccharomyces.

16. The process of claim 15, wherein the recombinant yeast host cell is from the species Saccharomyces cerevisiae.

17. The process of claim 1, wherein the biomass comprises starch or a starch derivative.

18. The process of claim 17, wherein the biomass is derived from or comprises corn, potato, cassava, rice, wheat, lignocellulosic material, milo or buckwheat.

19. The process of claim 18, wherein the biomass is derived from or comprises corn.

20. The process of claim 19, wherein the biomass comprises or is corn mash.

21. The process of claim 1, wherein the fermentation product is ethanol.

22. The process of claim 1, wherein the control dose allows achieving a fermentation yield of at least 0.415%, w/v per w/w of biomass.

23. The process of claim 1, wherein the reduced dose allows achieving a fermentation yield of at least 0.440%, w/v per w/w of biomass.

24. The process of claim 1, wherein the reduced dose of the exogenous enzyme is lower by at least 50%, 55%, 60%, 61%, 62%, 63%, 64%, 65%, 66%, 67%, 68%, 69%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% when compared to the control dose.

25. The process of claim 1 excluding including the exogenous enzyme in the biomass prior to or during fermentation.

26. The process of claim 1, wherein the reduced dose of the exogenous enzymes is reduced to zero.

27. The process of claim 1, wherein the fermentation yield is equal to or higher than the control fermentation yield of the control fermentation.

28. The process of claim 1, wherein the fermentation yield is substantially similar to the control fermentation yield of the control fermentation.

29. The process of claim 1, wherein the exogenous enzyme is a glucoamylase.

30. The process of claim 1 occurring at a commercial scale.
